(19) United States
(12) Patent Application Publication
(10) Pub. No.: US 2025/0074991 A1
(43) Pub. Date: Mar. 6, 2025

(54) METHODS OF TREATING NAUSEA AND VOMITING DISORDERS USING ANTI-GFRAL ANTIBODIES

(71) Applicant: NGM Biopharmaceuticals, Inc., South San Francisco, CA (US)

(72) Inventors: Alexander Mark DePaoli, Santa Barbara, CA (US); Carroll E. Henschel, Oakland, CA (US); Jer-Yuan Hsu, San Bruno, CA (US); Ursula B. Jeffry, San Francisco, CA (US)

(21) Appl. No.: 18/788,210

(22) Filed: Jul. 30, 2024

Related U.S. Application Data
(60) Provisional application No. 63/516,724, filed on Jul. 31, 2023.

(51) Int. Cl. C07K 16/28; A61K 39/00; A61P 1/00

(52) U.S. Cl. CPC C07K 16/2863; A61P 1/00; A61K 2039/505

(57) ABSTRACT

The present disclosure relates generally to methods of treating, preventing, or reducing nausea, vomiting, or a combination thereof, hyperemesis gravidarum (HG), nausea and vomiting of pregnancy (NVP), or sequelae thereof in a human subject in need thereof, the method comprising administering to the human subject a therapeutically effective amount of an antibody that specifically binds to human glial cell line-derived neurotrophic factor family receptor alpha-like (GFRAL).

### CROSS-REFERENCE TO RELATED APPLICATIONS

This application claims the benefit of U.S. Provisional Appl. No. 63/516,724, filed Jul. 31, 2023, the contents of which are incorporated by reference in their entirety herein.

### SEQUENCE LISTING

The instant application contains a Sequence Listing which has been submitted electronically in XML format and is hereby incorporated by reference in its entirety. Said XML copy, created on Jul. 26, 2024, is named 47702-0116001_SL.xml and is 848,585 bytes in size.

### I. FIELD

The present disclosure relates generally to methods of treating, preventing, or reducing nausea, vomiting, or a combination thereof, hyperemesis gravidarum (HG), nausea and vomiting of pregnancy (NVP), or sequelae thereof in a human subject in need thereof, the method comprising administering to the human subject a therapeutically effective amount of an antibody that specifically binds to human glial cell line-derived neurotrophic factor family receptor alpha-like (GFRAL).

### II. BACKGROUND OF THE INVENTION

Growth differentiation factor 15 (GDF15) is a protein belonging to the transforming growth factor beta (TGF-β) superfamily. GDF15 is also known as TGF-PL, MIC-1, PDF, PLAB, NAG-1, and PTGFB. GDF15 mRNA is reported to be most abundant in the liver, with lower levels seen in some other tissues. Its expression in liver can be significantly up-regulated in injury of organs such as liver, kidney, heart and lung. GDF15 also is upregulated during pregnancy and is produced in the placenta.

GDF15 is reported to play a role in regulating inflammatory and apoptotic pathways in injured tissues and during disease processes, including nausea and vomiting.

There is a significant unmet need for therapeutic agents effective in nausea and vomiting of pregnancy (NVP), hyperemesis gravidarum (HG), and other vomiting disorders.

### III. BRIEF SUMMARY OF THE INVENTION

Provided herein is a method for treating HG in a human subject in need thereof, the method comprising administering to the human subject a therapeutically effective amount of an antibody that specifically binds human GFRAL.

Also provided herein is a method for treating NVP in a human subject in need thereof, the method comprising administering to the human subject a therapeutically effective amount of an antibody that specifically binds human GFRAL. In some instances, the NVP is mild NVP. In other instances, the NVP is moderate NVP. In yet other instances, the NVP is severe NVP.

Also provided herein is a method for treating nausea and/or vomiting in a human subject in need thereof, the method comprising administering to the human subject a therapeutically effective amount of an antibody that specifically binds human GDNF Family Receptor Alpha-Like (GFRAL).

Also provided herein is a method for reducing nausea and/or vomiting in a human subject in need thereof, the method comprising administering to the human subject a therapeutically effective amount of an antibody that specifically binds human GDNF Family Receptor Alpha-Like (GFRAL).

In some instances, the administering is performed subcutaneously. In some instances, the administering is performed intravenously. In some instances, the methods above include administration of one or more of: doxylamine, pyridoxine, ondansetron, dopamine antagonists (e.g., metoclopramide, promethazine), diphenhydramine, droperidol, methylprednisolone, mirtazapine, steroid therapy, an anti-emetic, vitamin B1, folic acid, vitamin K, vitamin D, Magnesium, and ginger. In some instances, the methods above also include a previous therapy comprising administration to the human subject of one or more of: doxylamine, pyridoxine, ondansetron, dopamine antagonists (e.g., metoclopramide, promethazine), diphenhydramine, droperidol, methylprednisolone, mirtazapine, steroid therapy, an anti-emetic, vitamin B1, folic acid, vitamin K, vitamin D, Magnesium, and ginger. In some cases, the human subject does not respond to the previous therapy.

In some instances, the therapeutically effective amount of the antibody is about 100 mg, optionally wherein the therapeutically effective amount of the antibody is 100 mg.

In other instances, the therapeutically effective amount of the antibody is about 75 mg, optionally wherein the therapeutically effective amount of the antibody is 75 mg. In some cases, the therapeutically effective amount of the antibody is administered only once (a single dose) to the human subject. In other instances, the therapeutically effective amount of the antibody is administered twice or as needed to obtain a clinically-beneficial effect in the patient. In some cases, the therapeutically effective amount of the antibody is administered by subcutaneous injection. In yet other cases, the therapeutically effective amount of the antibody is administered by or intravenous injection.

In some instances, the therapeutically effective amount of the antibody is about 30 mg, optionally wherein the therapeutically effective amount of the antibody is 30 mg.

In some instances, the therapeutically effective amount of the antibody is about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 180 mg, about 200 mg, or more of the antibody. In other instances, the therapeutically effective amount of the antibody is 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 180 mg, or 200 mg of the antibody. In some cases, the therapeutically effective amount of the antibody is administered once, twice, or as needed to obtain a clinically-beneficial effect in the patient. In certain cases, the therapeutically effective amount of the antibody is administered by subcutaneous or intravenous injection.

In some instances, the method comprises administering more than one (e.g., 1, 2, 3, 4, 5, 6, 7, 8, 9, or 10 doses) dose of the therapeutically effective amount of the antibody to the human subject. Optionally, the therapeutically effective amount of the antibody is administered as needed to obtain a clinically-beneficial effect in the patient.

In some instances, the method comprises administering more than one dose of the therapeutically effective amount of the antibody to the human subject, wherein each does is administered with an interval of about one week, two weeks, three weeks, four weeks, five weeks, six weeks, or more, or on an as-needed basis (i.e., a variable interval) to obtain a clinically-beneficial effect in the patient.

In some instances, the method treats a subject diagnosed as having HG and the therapeutically effective amount of the antibody is administered only once during the pregnancy. In other instances, the therapeutically effective amount of the antibody is administered twice or three times during the pregnancy with the interval of about one week, two weeks, three weeks, four weeks, five weeks, six weeks, seven weeks, eight weeks, or more between the administrations. Optionally, the therapeutically effective amount of the antibody is administered as needed to obtain a clinically-beneficial effect in the patient.

In some instances, a biological sample obtained from the human subject has an elevated level of GDF15 compared to a control level of GDF15 in a control biological sample obtained from one or more humans not suffering from nausea and vomiting of pregnancy, hyperemesis gravidarum, nausea, or vomiting. In some instances, the biological sample is serum, plasma, or blood.

In some instances, the antibody inhibits binding of human GFRAL to human RET.

In some instances, the antibody does not inhibit binding of human GDF15 to human GFRAL.

In some instances, the antibody inhibits binding of human GFRAL to human RET and does not inhibit binding of human GDF15 to human GFRAL.

In some instances, the antibody inhibits binding of human GDF15 to human GFRAL.

In some instances, the antibody specifically binds within amino acid residues 220-316 of the amino acid sequence set forth in SEQ ID NO: 1797. In some instances, the antibody inhibits binding of human RET to human GFRAL. In some instances, the antibody does not inhibit binding of human GDF15 to human GFRAL. In some instances, the antibody inhibits binding of human RET to human GFRAL and the antibody does not inhibit binding of human GDF15 to human GFRAL.

In some instances, the antibody specifically binds to one or more residues selected from the group consisting of: Met214, Pro216, Pro217, Gln290, Cys291, Thr292, Cys293, Arg294, Thr295, Ile296, Thr297, Gln298, Ser299, Glu301, Lys305, Gln308, His309, His312, and Ser315 of the amino acid sequence set forth in SEQ ID NO: 1797. In some instances, the antibody inhibits binding of human RET to human GFRAL. In some instances, the antibody does not inhibit binding of human GDF15 to human GFRAL. In some instances, the antibody inhibits binding of human RET to human GFRAL and the antibody does not inhibit binding of human GDF15 to human GFRAL.

In some instances, the antibody specifically binds to Thr297, Gln298, and Ser299 of the amino acid sequence set forth in SEQ ID NO:1797.

In some instances, the antibody binds GFRAL and comprises a heavy chain variable region (VH) and a light chain variable region (VL), and wherein the VH comprises a VH complementarity determining region (CDR) 1, a VH CDR2, and a VH CDR3 from the amino acid sequence set forth in SEQ ID NO: 1982, and the VL comprises a VL CDR1, a VL CDR2, and a VL CDR3 from the amino acid sequence set forth in SEQ ID NO: 1997.

In some instances, the antibody binds GFRAL and comprises a VH and a VL, wherein the VH comprises a VH CDR1, a VH CDR2, and a VH CDR3, and the VL comprises a VL CDR1, a VL CDR2, and a VL CDR3, wherein the VH CDR1, VH CDR2, VH CDR3, VL CDR1, VL CDR2, and VL CDR3 comprise or consist of the amino acid sequences set forth in SEQ ID NO:46, SEQ ID NO:137, SEQ ID NO:225, SEQ ID NO:301, SEQ ID NO:376, and SEQ ID NO:426, respectively.

In some instances, the antibody binds GFRAL and comprises a VH and a VL, wherein the VH comprises a VH CDR1, a VH CDR2, and a VH CDR3, and the VL comprises a VL CDR1, a VL CDR2, and a VL CDR3, wherein the VH CDR1, VH CDR2, VH CDR3, VL CDR1, VL CDR2, and VL CDR3 comprise or consist of the amino acid sequences set forth in SEQ ID NO:47, SEQ ID NO:138, SEQ ID NO:226, SEQ ID NO:302, SEQ ID NO:377, and SEQ ID NO:426, respectively.

In some instances, the antibody binds GFRAL and comprises a VH and a VL, wherein the VH comprises a VH CDR1, a VH CDR2, and a VH CDR3, and the VL comprises a VL CDR1, a VL CDR2, and a VL CDR3, wherein the VH CDR1, VH CDR2, VH CDR3, VL CDR1, VL CDR2, and VL CDR3 comprise or consist of the amino acid sequences set forth in SEQ ID NO:48, SEQ ID NO: 137, SEQ ID NO:225, SEQ ID NO:301, SEQ ID NO:376, and SEQ ID NO:426, respectively.

In some instances, the antibody binds GFRAL and comprises a VH and a VL, wherein the VH comprises a VH CDR1, a VH CDR2, and a VH CDR3, and the VL comprises a VL CDR1, a VL CDR2, and a VL CDR3, wherein the VH CDR1, VH CDR2, VH CDR3, VL CDR1, VL CDR2, and VL CDR3 comprise or consist of the amino acid sequences set forth in SEQ ID NO:49, SEQ ID NO: 139, SEQ ID NO:227, SEQ ID NO:303, SEQ ID NO:377, and SEQ ID NO:427, respectively.

In some instances, the antibody binds GFRAL and comprises a VH and a VL, wherein the VH comprises a VH CDR1, a VH CDR2, and a VH CDR3, and the VL comprises a VL CDR1, a VL CDR2, and a VL CDR3, wherein the VH CDR1, VH CDR2, VH CDR3, VL CDR1, VL CDR2, and VL CDR3 comprise or consist of the amino acid sequences set forth in SEQ ID NO:50, SEQ ID NO:140, SEQ ID NO:228, SEQ ID NO:304, SEQ ID NO:378, and SEQ ID NO:428, respectively.

In some instances, the antibody binds GFRAL and comprises a VH and a VL, wherein the VH comprises a VH CDR1, a VH CDR2, and a VH CDR3, and the VL comprises a VL CDR1, a VL CDR2, and a VL CDR3, wherein the VH CDR1, VH CDR2, VH CDR3, VL CDR1, VL CDR2, and VL CDR3 comprise or consist of the amino acid sequences set forth in SEQ ID NO:46, SEQ ID NO: 141, SEQ ID NO:225, SEQ ID NO:301, SEQ ID NO:376, and SEQ ID NO:426, respectively.

In some instances, the antibody binds GFRAL and comprises a VH and a VL, wherein the VH comprises or consists of an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence set forth in any one of SEQ ID NOs:1978-1988.

In some instances, the antibody binds GFRAL and comprises a VH and a VL, wherein the VL comprises or consists of an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence set forth in any one of SEQ ID NOs:1990-2000.

In some instances, the antibody binds GFRAL and comprises a VH and a VL, wherein the VH comprises or consists of an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence set forth in any one of SEQ ID NOs:1978-1988 and the VL comprises an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence set forth in any one of SEQ ID NOs:1990-2000.

In some instances, the antibody comprises a VH and a VL, wherein the VH comprises or consists of the amino acid sequence set forth in any one of SEQ ID NOs:1978-1988.

In some instances, the antibody comprises a VH and a VL, wherein the VL comprises or consists of the amino acid sequence set forth in any one of SEQ ID NOs:1990-2000.

In some instances, the antibody binds GFRAL and comprises a VH and a VL, wherein the VH comprises or consists of an amino acid sequence having at least 80%, at least 85%, at least 90%, at least 91%, at least 92%, at least 93%, at least 94%, at least 95% sequence identity, at least 96%, at least 97%, at least 98%, at least 99%, or 100% identity to the amino acid sequence set forth in the amino acid sequence set forth in SEQ ID NO:1982. In some instances, the antibody comprises a VH and a VL, wherein the VH comprises or consists of the amino acid sequence set forth in SEQ ID NO:1982.

In some instances, the antibody binds GFRAL and comprises a VH and a VL, wherein the VL comprises or consists of an amino acid sequence having at least 80%, at least 85%, at least 90%, at least 91%, at least 92%, at least 93%, at least 94%, at least 95% sequence identity, at least 96%, at least 97%, at least 98%, at least 99%, or 100% identity to the amino acid sequence set forth in the amino acid sequence set forth in SEQ ID NO:1997. In some instances, the antibody comprises a VH and a VL, wherein the VL comprises or consists of the amino acid sequence set forth in SEQ ID NO:1997.

In some instances, the antibody binds GFRAL and comprises a VH and a VL, wherein the VH comprises or consists of an amino acid sequence having at least 80%, at least 85%, at least 90%, at least 91%, at least 92%, at least 93%, at least 94%, at least 95% sequence identity, at least 96%, at least 97%, at least 98%, or at least 99%, sequence identity to the amino acid sequence set forth in SEQ ID NO:3.

In some instances, the antibody binds GFRAL and comprises a VH and a VL, wherein the VL comprises or consists of an amino acid sequence having at least 80%, at least 85%, at least 90%, at least 91%, at least 92%, at least 93%, at least 94%, at least 95% sequence identity, at least 96%, at least 97%, at least 98%, or at least 99%, sequence identity to the amino acid sequence set forth in SEQ ID NO:4.

In some instances, the antibody binds GFRAL and comprises a VH and a VL, wherein the VH comprises or consists of an amino acid sequence having at least 80%, at least 85%, at least 90%, at least 91%, at least 92%, at least 93%, at least 94%, at least 95% sequence identity, at least 96%, at least 97%, at least 98%, or at least 99%, sequence identity to the amino acid sequence set forth in SEQ ID NO:3 and the VL comprises or consists of an amino acid sequence having at least 80%, at least 85%, at least 90%, at least 91%, at least 92%, at least 93%, at least 94%, at least 95% sequence identity, at least 96%, at least 97%, at least 98%, or at least 99%, sequence identity to the amino acid sequence set forth in SEQ ID NO: 4.

In some instances, the antibody comprises a VH and a VL, wherein the VH comprises or consists of the amino acid sequence set forth in SEQ ID NO:3.

In some instances, the antibody comprises a VH and a VL, wherein the VL comprises or consists of the amino acid sequence set forth in SEQ ID NO:4.

In some instances, the antibody comprises a VH and a VL, wherein the VH comprises or consists of the amino acid sequence set forth in SEQ ID NO:3 and the VL comprises the amino acid sequence set forth in SEQ ID NO:4.

In some instances, the antibody comprises a VH and a VL, wherein the VH comprises or consists of the amino acid sequence set forth in SEQ ID NO: 1982 and the VL comprises or consists of the amino acid sequence set forth in SEQ ID NO:1997.

In some instances, the antibody binds GFRAL and comprises a VH and a VL, wherein the VH comprises a VH CDR1, a VH CDR2, and a VH CDR3 from the amino acid sequence set forth in any one of SEQ ID NOs:7 and 1957-1965, and the VL comprises a VL CDR1, a VL CDR2, and a VL CDR3 from the amino acid sequence set forth in any one of SEQ ID NOs:8 and 1967-1976. In some instances, the antibody comprises a VH and a VL, wherein the VH comprises a VH CDR1, a VH CDR2, and a VH CDR3, and the VL comprises a VL CDR1, a VL CDR2, and a VL CDR3, wherein the VH CDR1, the VH CDR2, the VH CDR3, the VL CDR1, the VL CDR2, and the VL CDR3 comprise or consist of the amino acid sequences set forth in (i) SEQ ID NO: 56, SEQ ID NO: 147, SEQ ID NO: 233, SEQ ID NO: 309, SEQ ID NO: 382, and SEQ ID NO: 432, respectively; (ii) SEQ ID NO: 57, SEQ ID NO: 148, SEQ ID NO: 234, SEQ ID NO: 310, SEQ ID NO: 383, and SEQ ID NO: 432, respectively; (iii) SEQ ID NO: 58, SEQ ID NO: 147, SEQ ID NO: 233, SEQ ID NO: 309, SEQ ID NO: 382, and SEQ ID NO: 432, respectively; (iv) SEQ ID NO: 49, SEQ ID NO: 149, SEQ ID NO: 235, SEQ ID NO: 311, SEQ ID NO: 383, and SEQ ID NO: 433, respectively; (v) SEQ ID NO: 59, SEQ ID NO: 150, SEQ ID NO: 236, SEQ ID NO: 312, SEQ ID NO: 384, and SEQ ID NO: 434, respectively; or (vi) SEQ ID NO: 56, SEQ ID NO: 151, SEQ ID NO: 233, SEQ ID NO: 309, SEQ ID NO: 382, and SEQ ID NO: 432, respectively. In some instances, the VH comprises or consists of an amino acid sequence having at least 80%, at least 85%, at least 90%, or at least 95% sequence identity to the amino acid sequence set forth in any one of SEQ ID NOs:7 and 1957-1965 and the VL comprises or consists of an amino acid sequence having at least 80%, at least 85%, at least 90%, or at least 95% sequence identity to the amino acid sequence set forth in any one of SEQ ID NOs:8 and 1967-1976. In some instances, the VH comprises or consists of the amino acid sequence of in any one of SEQ ID NOs:7 and 1957-1965 and the VL comprises or consists of the amino acid sequence of in any one of SEQ ID NOs:8 and 1967-1976.

In some instances, the antibody binds GFRAL and comprises a VH and a VL, wherein the VH comprises a VH CDR1, a VH CDR2, and a VH CDR3 from the amino acid sequence set forth in SEQ ID NO:21, and the VL comprises a VL CDR1, a VL CDR2, and a VL CDR3 from the amino acid sequence set forth in SEQ ID NO:22. In some instances, the antibody comprises a VH and a VL, wherein the VH comprises a VH CDR1, a VH CDR2, and a VH CDR3, and the VL comprises a VL CDR1, a VL CDR2, and a VL CDR3, wherein the VH CDR1, the VH CDR2, the VH CDR3, the VL CDR1, the VL CDR2, and the VL CDR3 comprise or consist of the amino acid sequences set forth in (i) SEQ ID NO: 86, SEQ ID NO: 177, SEQ ID NO: 261, SEQ ID NO: 337, SEQ ID NO: 401, and SEQ ID NO: 453, respectively; (ii) SEQ ID NO: 87, SEQ ID NO: 178, SEQ ID NO: 262, SEQ ID NO: 338, SEQ ID NO: 386, and SEQ ID NO: 453, respectively; (iii) SEQ ID NO: 88, SEQ ID NO: 179, SEQ ID NO: 261, SEQ ID NO: 337, SEQ ID NO: 401, and SEQ ID NO: 453, respectively; (iv) SEQ ID NO: 89, SEQ ID NO: 168, SEQ ID NO: 263, SEQ ID NO: 339, SEQ ID NO: 386, and SEQ ID NO: 454, respectively; (v) SEQ ID NO: 90, SEQ ID NO: 180, SEQ ID NO: 264, SEQ ID NO: 340, SEQ ID NO: 402, and SEQ ID NO: 455, respectively; or (vi) SEQ ID NO: 86, SEQ ID NO: 181, SEQ ID NO: 261, SEQ ID NO: 337, SEQ ID NO: 401, and SEQ ID NO: 453, respectively. In some instances, the VH comprises or consists of an amino acid sequence having at least 80%, at least 85%, at least 90%, or at least 95% sequence identity to the amino acid sequence set forth in SEQ ID NO:21 and the VL comprises or consists of an amino acid sequence having at least 80%, at least 85%, at least 90%, or at least 95% sequence identity to the amino acid sequence set forth in SEQ ID NO: 22. In some instances, the VH comprises or consists of the amino acid sequence of SEQ ID NO: 21 and the VL comprises or consists of the amino acid sequence of SEQ ID NO:22.

In some instances, the antibody binds GFRAL and comprises a VH and a VL, wherein the VH comprises a VH CDR1, a VH CDR2, and a VH CDR3 from the amino acid sequence set forth in SEQ ID NO:23, and the VL comprises a VL CDR1, a VL CDR2, and a VL CDR3 from the amino acid sequence set forth in SEQ ID NO:24. In some instances, the antibody comprises a VH and a VL, wherein the VH comprises a VH CDR1, a VH CDR2, and a VH CDR3, and the VL comprises a VL CDR1, a VL CDR2, and a VL CDR3, wherein the VH CDR1, the VH CDR2, the VH CDR3, the VL CDR1, the VL CDR2, and the VL CDR3 comprise or consist of the amino acid sequences set forth in (i) SEQ ID NO: 91, SEQ ID NO: 182, SEQ ID NO: 265, SEQ ID NO: 341, SEQ ID NO: 385, and SEQ ID NO: 456, respectively; (ii) SEQ ID NO: 92, SEQ ID NO: 183, SEQ ID NO: 266, SEQ ID NO: 342, SEQ ID NO: 386, and SEQ ID NO: 456, respectively; (iii) SEQ ID NO: 93, SEQ ID NO: 182, SEQ ID NO: 265, SEQ ID NO: 341, SEQ ID NO: 385, and SEQ ID NO: 456, respectively; (iv) SEQ ID NO: 75, SEQ ID NO: 184, SEQ ID NO: 267, SEQ ID NO: 343, SEQ ID NO: 386, and SEQ ID NO: 457, respectively; (v) SEQ ID NO: 94, SEQ ID NO: 185, SEQ ID NO: 268, SEQ ID NO: 344, SEQ ID NO: 403, and SEQ ID NO: 458, respectively; or (vi) SEQ ID NO: 91, SEQ ID NO: 186, SEQ ID NO: 265, SEQ ID NO: 341, SEQ ID NO: 385, and SEQ ID NO: 456, respectively. In some instances, the VH comprises or consists of an amino acid sequence having at least 80%, at least 85%, at least 90%, or at least 95% sequence identity to the amino acid sequence set forth in SEQ ID NO:23 and the VL comprises or consists of an amino acid sequence having at least 80%, at least 85%, at least 90%, or at least 95% sequence identity to the amino acid sequence set forth in SEQ ID NO: 24. In some instances, the VH comprises or consists of the amino acid sequence of SEQ ID NO: 23 and the VL comprises or consists of the amino acid sequence of SEQ ID NO:24.

In some instances, the antibody binds GFRAL and comprises a VH and a VL, wherein the VH comprises a VH CDR1, a VH CDR2, and a VH CDR3 from the amino acid sequence set forth in SEQ ID NO:25, and the VL comprises a VL CDR1, a VL CDR2, and a VL CDR3 from the amino acid sequence set forth in SEQ ID NO:26. In some instances, the antibody comprises a VH and a VL, wherein the VH comprises a VH CDR1, a VH CDR2, and a VH CDR3, and the VL comprises a VL CDR1, a VL CDR2, and a VL CDR3, wherein the VH CDR1, the VH CDR2, the VH CDR3, the VL CDR1, the VL CDR2, and the VL CDR3 comprise or consist of the amino acid sequences set forth in (i) SEQ ID NO: 95, SEQ ID NO: 187, SEQ ID NO: 269, SEQ ID NO: 345, SEQ ID NO: 404, and SEQ ID NO: 459, respectively; (ii) SEQ ID NO: 96, SEQ ID NO: 188, SEQ ID NO: 270, SEQ ID NO: 346, SEQ ID NO: 405, and SEQ ID NO: 459, respectively; (iii) SEQ ID NO: 97, SEQ ID NO: 187, SEQ ID NO: 269, SEQ ID NO: 345, SEQ ID NO: 404, and SEQ ID NO: 459, respectively; (iv) SEQ ID NO: 98, SEQ ID NO: 184, SEQ ID NO: 271, SEQ ID NO: 347, SEQ ID NO: 405, and SEQ ID NO: 460, respectively; (v) SEQ ID NO: 99, SEQ ID NO: 189, SEQ ID NO: 272, SEQ ID NO: 348, SEQ ID NO: 406, and SEQ ID NO: 461, respectively; or (vi) SEQ ID NO: 95, SEQ ID NO: 190, SEQ ID NO: 269, SEQ ID NO: 345, SEQ ID NO: 404, and SEQ ID NO: 459, respectively. In some instances, the VH comprises or consists of an amino acid sequence having at least 80%, at least 85%, at least 90%, or at least 95% sequence identity to the amino acid sequence set forth in SEQ ID NO:25 and the VL comprises or consists of an amino acid sequence having at least 80%, at least 85%, at least 90%, or at least 95% sequence identity to the amino acid sequence set forth in SEQ ID NO: 26. In some instances, the VH comprises or consists of the amino acid sequence of SEQ ID NO: 25 and the VL comprises or consists of the amino acid sequence of SEQ ID NO:26.

In some instances, the antibody binds GFRAL and comprises a VH and a VL, wherein the VH comprises a VH CDR1, a VH CDR2, and a VH CDR3 from the amino acid sequence set forth in SEQ ID NO:37, and the VL comprises a VL CDR1, a VL CDR2, and a VL CDR3 from the amino acid sequence set forth in SEQ ID NO:38. In some instances, the antibody comprises a VH and a VL, wherein the VH comprises a VH CDR1, a VH CDR2, and a VH CDR3, and the VL comprises a VL CDR1, a VL CDR2, and a VL CDR3, wherein the VH CDR1, the VH CDR2, the VH CDR3, the VL CDR1, the VL CDR2, and the VL CDR3 comprise or consist of the amino acid sequences set forth in (i) SEQ ID NO: 124, SEQ ID NO: 210, SEQ ID NO: 289, SEQ ID NO: 365, SEQ ID NO: 418, and SEQ ID NO: 474, respectively; (ii) SEQ ID NO: 73, SEQ ID NO: 211, SEQ ID NO: 290, SEQ ID NO: 366, SEQ ID NO: 419, and SEQ ID NO: 474, respectively; (iii) SEQ ID NO: 125, SEQ ID NO: 210, SEQ ID NO: 289, SEQ ID NO: 365, SEQ ID NO: 418, and SEQ ID NO: 474, respectively; (iv) SEQ ID NO: 75, SEQ ID NO: 212, SEQ ID NO: 291, SEQ ID NO: 367, SEQ ID NO: 419, and SEQ ID NO: 475, respectively; (v) SEQ ID NO: 126, SEQ ID NO: 213, SEQ ID NO: 292, SEQ ID NO: 368, SEQ ID NO: 420, and SEQ ID NO: 476, respectively; or (vi) SEQ ID NO: 124, SEQ ID NO: 214, SEQ ID NO: 289, SEQ ID NO: 365, SEQ ID NO: 418, and SEQ ID NO: 474, respectively. In some instances, the VH comprises or consists of an amino acid sequence having at least 80%, at least 85%, at least 90%, or at least 95% sequence identity to the amino acid sequence set forth in SEQ ID NO:37 and the VL comprises or consists of an amino acid sequence having at least 80%, at least 85%, at least 90%, or at least 95% sequence identity to the amino acid sequence set forth in SEQ ID NO: 38. In some instances, the VH comprises or consists of the amino acid sequence of SEQ ID NO: 37 and the VL comprises or consists of the amino acid sequence of SEQ ID NO:38.

In some instances, the VH comprises or consists of the amino acid sequence of SEQ ID NO:39 and the VL comprises or consists of the amino acid sequence of SEQ ID NO: 40. In some instances, the antibody comprises a VH and a VL, wherein the VH comprises a VH CDR1, a VH CDR2, and a VH CDR3 from the amino acid sequence set forth in SEQ ID NO:39, and the VL comprises a VL CDR1, a VL CDR2, and a VL CDR3 from the amino acid sequence set forth in SEQ ID NO:40. In some instances, the antibody comprises a VH and a VL, wherein the VH comprises a VH CDR1, a VH CDR2, and a VH CDR3, and the VL comprises a VL CDR1, a VL CDR2, and a VL CDR3, wherein (i) the VH CDR1, the VH CDR2, the VH CDR3, the VL CDR1, the VL CDR2, and the VL CDR3 comprise or consist of the amino acid sequences set forth in SEQ ID NO: 127, SEQ ID NO: 215, SEQ ID NO: 293, SEQ ID NO: 369, SEQ ID NO: 421, and SEQ ID NO: 477, respectively; (ii) the VH CDR1, the VH CDR2, the VH CDR3, the VL CDR1, the VL CDR2, and the VL CDR3 comprise the amino acid sequences set forth in SEQ ID NO: 128, SEQ ID NO: 216, SEQ ID NO: 294, SEQ ID NO: 370, SEQ ID NO: 405, and SEQ ID NO: 477, respectively; (iii) the VH CDR1, the VH CDR2, the VH CDR3, the VL CDR1, the VL CDR2, and the VL CDR3 comprise the amino acid sequences set forth in SEQ ID NO: 129, SEQ ID NO: 215, SEQ ID NO: 293, SEQ ID NO: 369, SEQ ID NO: 421, and SEQ ID NO: 477, respectively; (iv) the VH CDR1, the VH CDR2, the VH CDR3, the VL CDR1, the VL CDR2, and the VL CDR3 comprise the amino acid sequences set forth in SEQ ID NO: 130, SEQ ID NO: 217, SEQ ID NO: 295, SEQ ID NO: 371, SEQ ID NO: 405, and SEQ ID NO: 478, respectively; (v) the VH CDR1, the VH CDR2, the VH CDR3, the VL CDR1, the VL CDR2, and the VL CDR3 comprise or consist of the amino acid sequences set forth in SEQ ID NO: 131, SEQ ID NO: 218, SEQ ID NO: 296, SEQ ID NO: 372, SEQ ID NO: 422, and SEQ ID NO: 479, respectively; or (vi) the VH CDR1, the VH CDR2, the VH CDR3, the VL CDR1, the VL CDR2, and the VL CDR3 comprise the amino acid sequences set forth in SEQ ID NO: 127, SEQ ID NO: 219, SEQ ID NO: 293, SEQ ID NO: 369, SEQ ID NO: 421, and SEQ ID NO: 477, respectively. In some instances, the VH comprises or consists of an amino acid sequence having at least 80%, at least 85%, at least 90%, or at least 95% sequence identity to the amino acid sequence set forth in SEQ ID NO:39 and the VL comprises or consists of an amino acid sequence having at least 80%, at least 85%, at least 90%, or at least 95% sequence identity to the amino acid sequence set forth in SEQ ID NO:40.

In some instances, the antibody specifically binds within amino acid residues 20-130 of the amino acid sequence set forth in SEQ ID NO: 1797. In some instances, the antibody does not inhibit binding of human GDF15 to human GFRAL.

In some instances, the antibody specifically binds within amino acid residues 131-210 of the amino acid sequence set forth in SEQ ID NO: 1797. In some instances, the antibody inhibits binding of human GDF15 to human GFRAL.

In some instances, the antibody specifically binds to: (a) one or more residues selected from the group consisting of GLY140, LEU148, ALA149, ALA146, VAL142, ASN145, VAL139, ALA135, GLU136, LEU152, LEU132, SER201, ALA204, LEU205, LYS153, ILE196, PRO197, and GLN200 of a GFRAL protein (SEQ ID NO: 1797), and optionally wherein the antibody inhibits binding of the GFRAL protein to a GDF15 protein; (b) one or more residues selected from the group consisting of SER156, GLN147, SER150, TYR151, ALA154, CYS155, PHE174, TYR175, ALA137, CYS138, ASP141, VAL143, CYS144, LEU186, CYS189, CYS191, ALA192, GLN193, SER194, ASP195, CYS198, GLN199, LYS202, GLU203, HIS206, SER207, SER 130, CYS131, LEU132, GLU133, and VAL134 of a GFRAL protein (SEQ ID NO: 1797), and optionally wherein the antibody inhibits binding of the GFRAL protein to a GDF15 protein; (c) one or more residues selected from the group consisting of SER156, GLN147, LEU148, ALA149, SER150, TYR151, LEU152, LYS153, ALA154, CYS155, PHE174, TYR175, GLU136, ALA137, CYS138, VAL139, GLY140, ASP141, VAL142, VAL143, CYS144, ASN145, ALA146, LEU186, CYS189, CYS191, ALA192, GLN193, SER194, ASP195, ILE196, PRO197, CYS198, GLN199, GLN200, SER201, LYS202, GLU203, ALA204, LEU205, HIS206, SER207, SER130, CYS131, LEU132, GLU133, VAL134, and ALA135 of a GFRAL protein (SEQ ID NO: 1797), and optionally wherein the antibody inhibits binding of the GFRAL protein to a GDF15 protein; (d) one or more residues selected from the group consisting of GLU136, ALA137, VAL139, GLY140, ASP141, VAL142, VAL143, CYS144, ASN145, ALA146, GLN147, PHE173, ASN177, ILE178, PRO179, ASN181, ILE182, and MET185 of a GFRAL protein (SEQ ID NO: 1797), and optionally wherein the antibody inhibits binding of the GFRAL protein to a GDF15 protein; (c) one or more residues selected from the group consisting of LEU132, GLU133, VAL134, ALA135, CYS138, LEU148, ALA149, SER150, TYR151, PHE174, TYR175, ALA169, ALA170, ILE171, ARG172, GLN176, PHE180, ALA183, GLN184, LEU186, ALA187, PHE188, and CYS189 of a GFRAL protein (SEQ ID NO: 1797), and optionally wherein the antibody inhibits binding of the GFRAL protein to a GDF15 protein; (f) one or more residues selected from the group consisting of LEU132, GLU133, VAL134, ALA135, GLU136, ALA137, CYS138, VAL139, GLY140, ASP141, VAL142, VAL143, CYS144, ASN145, ALA146, GLN147, LEU148, ALA149, SER150, TYR151, PHE174, TYR175, ALA169, ALA170, ILE171, ARG172, PHE173, GLN176, ASN177, ILE178, PRO179, PHE180, ASN181, ILE182, ALA183, GLN184, MET185, LEU186, ALA187, PHE188, and CYS189 of a GFRAL protein (SEQ ID NO: 1797), and optionally wherein the antibody inhibits binding of the GFRAL protein to a GDF15 protein; or (g) one or more residues selected from the group consisting of MET214, PRO216, PRO217, GLN290, CYS291, THR292, CYS293, ARG294, THR295, ILE296, THR297, GLN298, SER299, GLU301, LYS305, GLN308, HIS309, HIS312, and SER315 of the GFRAL protein (SEQ ID NO: 1797), and optionally wherein the antibody inhibits binding of the GFRAL protein to a RET protein.

In some instances, the antibody comprises a VH and a VL, and wherein: (a) the VH comprises a VH CDR1, a VH CDR2, and a VH CDR3 from the amino acid sequence set forth in any one of SEQ ID NOs:15 and 1936-1941, and the VL comprises a VL CDR1, a VL CDR2, and a VL CDR3 from the amino acid sequence set forth in any one of SEQ ID NOs:16 and 1943-1947; (b) the VH comprises a VH CDR1, a VH CDR2, and a VH CDR3 from the amino acid sequence set forth in any one of SEQ ID NOs:1 and 1918-1927, and the VL comprises a VL CDR1, a VL CDR2, and a VL CDR3 from the amino acid sequence set forth in any one of SEQ ID NOs:2 and 1929-1934; or (c) the VH comprises a VH CDR1, a VH CDR2, and a VH CDR3 from the amino acid sequence set forth in SEQ ID NO:11, and the VL comprises a VL CDR1, a VL CDR2, and a VL CDR3 from the amino acid sequence set forth in SEQ ID NO:12; (c) the VH comprises a VH CDR1, a VH CDR2, and a VH CDR3 from the amino acid sequence set forth in SEQ ID NO:5, and the VL comprises a VL CDR1, a VL CDR2, and a VL CDR3 from the amino acid sequence set forth in SEQ ID NO: 6; (c) the VH comprises a VH CDR1, a VH CDR2, and a VH CDR3 from the amino acid sequence set forth in SEQ ID NO:9, and the VL comprises a VL CDR1, a VL CDR2, and a VL CDR3 from the amino acid sequence set forth in SEQ ID NO: 10; (g) the VH comprises a VH CDR1, a VH CDR2, and a VH CDR3 from the amino acid sequence set forth in SEQ ID NO: 13, and the VL comprises a VL CDR1, a VL CDR2, and a VL CDR3 from the amino acid sequence set forth in SEQ ID NO: 14; (i) the VH comprises a VH CDR1, a VH CDR2, and a VH CDR3 from the amino acid sequence set forth in SEQ ID NO: 17, and the VL comprises a VL CDR1, a VL CDR2, and a VL CDR3 from the amino acid sequence set forth in SEQ ID NO:18; (j) the VH comprises a VH CDR1, a VH CDR2, and a VH CDR3 from the amino acid sequence set forth in SEQ ID NO: 19, and the VL comprises a VL CDR1, a VL CDR2, and a VL CDR3 from the amino acid sequence set forth in SEQ ID NO:20; (n) the VH comprises a VH CDR1, a VH CDR2, and a VH CDR3 from the amino acid sequence set forth in SEQ ID NO:27, and the VL comprises a VL CDR1, a VL CDR2, and a VL CDR3 from the amino acid sequence set forth in SEQ ID NO:28; (0) the VH comprises a VH CDR1, a VH CDR2, and a VH CDR3 from the amino acid sequence set forth in SEQ ID NO:29, and the VL comprises a VL CDR1, a VL CDR2, and a VL CDR3 from the amino acid sequence set forth in SEQ ID NO:30; (p) the VH comprises a VH CDR1, a VH CDR2, and a VH CDR3 from the amino acid sequence set forth in SEQ ID NO:31, and the VL comprises a VL CDR1, a VL CDR2, and a VL CDR3 from the amino acid sequence set forth in SEQ ID NO: 32; (q) the VH comprises a VH CDR1, a VH CDR2, and a VH CDR3 from the amino acid sequence set forth in SEQ ID NO:33, and the VL comprises a VL CDR1, a VL CDR2, and a VL CDR3 from the amino acid sequence set forth in SEQ ID NO:34; (r) the VH comprises a VH CDR1, a VH CDR2, and a VH CDR3 from the amino acid sequence set forth in SEQ ID NO:35, and the VL comprises a VL CDR1, a VL CDR2, and a VL CDR3 from the amino acid sequence set forth in SEQ ID NO:36; (u) the VH comprises a VH CDR1, a VH CDR2, and a VH CDR3 from the amino acid sequence set forth in SEQ ID NO:480, and the VL comprises a VL CDR1, a VL CDR2, and a VL CDR3 from the amino acid sequence set forth in SEQ ID NO:481; (v) the VH comprises a VH CDR1, a VH CDR2, and a VH CDR3 from the amino acid sequence set forth in SEQ ID NO:482, and the VL comprises a VL CDR1, a VL CDR2, and a VL CDR3 from the amino acid sequence set forth in SEQ ID NO: 483; (w) the VH comprises a VH CDR1, a VH CDR2, and a VH CDR3 from the amino acid sequence set forth in SEQ ID NO:484, and the VL comprises a VL CDR1, a VL CDR2, and a VL CDR3 from the amino acid sequence set forth in SEQ ID NO:485; or (x) the VH comprises a VH CDR1, a VH CDR2, and a VH CDR3 from the amino acid sequence set forth in SEQ ID NO:486, and the VL comprises a VL CDR1, a VL CDR2, and a VL CDR3 from the amino acid sequence set forth in SEQ ID NO:487.

In some instances, the antibody is an antibody that competes with an antibody of any one of Tables 1-24 for binding to human GFRAL. In one instance, the antibody is an antibody that competes with 3P10. In one instance, the antibody is an antibody that competes with Hz3p10. In one instance, the antibody is an antibody that competes with 5F12. In one instance, the antibody is an antibody that competes with a humanized 5F12 antibody.

In some instances, the antibody comprises two heavy chains and two light chains.

In some instances, the antibody is a human IgG1, human IgG2, or human IgG4 antibody.

In some instances, the antibody is a human IgG1 antibody.

In some instances, the antibody comprises a human kappa light chain constant region.

In some instances, the antibody comprises a human lambda light chain constant region.

In some instances, the antibody is a Fab, Fab′, F(ab′)₂, Fv, scFv, (scFv)₂, single chain antibody, dual variable region antibody, single variable region antibody, linear antibody, diabody, nanobody, or a V region antibody.

In some instances, the antibody binds GFRAL and comprises a heavy chain comprising an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 2009 or 2010 and a light chain comprising an amino acid sequence having at least 80%, at least 85%, at least 90%, at least 91%, at least 92%, at least 93%, at least 94%, at least 95%, at least 96%, at least 97%, at least 98%, at least 99%, or 100% sequence identity to the amino acid sequence set forth in SEQ ID NO: 2011 or 2012.

In some instances, the antibody comprises a heavy chain comprising the amino acid sequence set forth in SEQ ID NO: 2010 and a light chain comprising the amino acid sequence set forth in SEQ ID NO: 2012.

In some instances, the antibody comprises a heavy chain comprising the amino acid sequence set forth in SEQ ID NO: 2009 and a light chain comprising the amino acid sequence set forth in SEQ ID NO: 2011.

In some instances, the antibody comprises a heavy chain consisting of the amino acid sequence set forth in SEQ ID NO: 2010 and a light chain consisting of the amino acid sequence set forth in SEQ ID NO: 2012.

In some instances, the antibody comprises a heavy chain consisting of the amino acid sequence set forth in SEQ ID NO: 2009 and a light chain consisting of the amino acid sequence set forth in SEQ ID NO: 2011.

In some instances, the antibody is humanized.

In some instances, the human subject (a) is unresponsive to prior administration of an antiemetic or (b) has unresolved nausea, vomiting, or a combination thereof after prior administration of an antiemetic.

Also provided herein is a method for treating HG in a human subject in need thereof, the method comprising administering to the human subject a therapeutically effective amount of an antibody that specifically binds human GFRAL. In some instances, the antibody comprises (i) a VH comprising the VH CDR1, the VH CDR2, the VH CDR3 from the amino acid sequence set forth in SEQ ID NO: 1982; and (ii) a VL comprising the VL CDR1, the VL CDR2, and the VL CDR3 from the amino acid sequence set forth in SEQ ID NO: 1997. In some instances, the antibody comprises: (i) a VH comprising a VH CDR1 comprising or consisting of the amino acid sequence set forth in SEQ ID NO:48, a VH CDR2 comprising or consisting of the amino acid sequence set forth in SEQ ID NO: 137, and a VH CDR3 comprising or consisting of the amino acid sequence set forth in SEQ ID NO:225; and (ii) a VL comprising a VL CDR1 comprising or consisting of the amino acid sequence set forth in SEQ ID NO:301, a VL CDR2 comprising or consisting of the amino acid sequence set forth in SEQ ID NO:376, and a VL CDR3 comprising or consisting of the amino acid sequence set forth in SEQ ID NO:426. In some instances, the antibody comprises: (i) a VH comprising or consisting of the amino acid sequence set forth in SEQ ID NO:1982; and (ii) a VL comprising or consisting of the amino acid sequence set forth in SEQ ID NO:1997. In some instances, the antibody comprises: (i) a heavy chain comprising or consisting of the amino acid sequence set forth in SEQ ID NO:2010; and (ii) a light chain comprising or consisting of the amino acid sequence set forth in SEQ ID NO:2012. In some instances, the therapeutically effective amount of the antibody is administered as a single dose. In some instances, the single dose is 30 mg. In some instances, the single dose is 75 mg. In some instances, the single dose is 100 mg. In certain instances, the dose is administered subcutaneously. In other cases, the dose is administered intravenously. In some instances, the single dose is administered during gestational week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36.

Also provided herein is a method for treating NVP in a human subject in need thereof, the method comprising administering to the human subject a therapeutically effective amount of an antibody that specifically binds human GFRAL. In some instances, the antibody comprises (i) a VH comprising the VH CDR1, the VH CDR2, the VH CDR3 from the amino acid sequence set forth in SEQ ID NO: 1982; and (ii) a VL comprising the VL CDR1, the VL CDR2, and the VL CDR3 from the amino acid sequence set forth in SEQ ID NO: 1997. In some instances, the antibody comprises: (i) a VH comprising a VH CDR1 comprising or consisting of the amino acid sequence set forth in SEQ ID NO:48, a VH CDR2 comprising or consisting of the amino acid sequence set forth in SEQ ID NO: 137, and a VH CDR3 comprising or consisting of the amino acid sequence set forth in SEQ ID NO:225; and (ii) a VL comprising a VL CDR1 comprising or consisting of the amino acid sequence set forth in SEQ ID NO:301, a VL CDR2 comprising or consisting of the amino acid sequence set forth in SEQ ID NO:376, and a VL CDR3 comprising or consisting of the amino acid sequence set forth in SEQ ID NO:426. In some instances, the antibody comprises: (i) a VH comprising or consisting of the amino acid sequence set forth in SEQ ID NO: 1982; and (ii) a VL comprising or consisting of the amino acid sequence set forth in SEQ ID NO:1997. In some instances, the antibody comprises: (i) a heavy chain comprising or consisting of the amino acid sequence set forth in SEQ ID NO:2010; and (ii) a light chain comprising or consisting of the amino acid sequence set forth in SEQ ID NO:2012. In some instances, the therapeutically effective amount of the antibody is administered as a single dose. In some instances, the single dose is 30 mg. In some instances, the single dose is 75 mg. In some instances, the single dose is 100 mg. In certain instances, the dose is administered subcutaneously. In other cases, the dose is administered intravenously. In some instances, the single dose is administered during gestational week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36.

Also provided herein is a method for treating nausea and/or vomiting in a human subject in need thereof, the method comprising administering to the human subject a therapeutically effective amount of an antibody that specifically binds human GFRAL. In some instances, the antibody comprises (i) a VH comprising the VH CDR1, the VH CDR2, the VH CDR3 from the amino acid sequence set forth in SEQ ID NO: 1982; and (ii) a VL comprising the VL CDR1, the VL CDR2, and the VL CDR3 from the amino acid sequence set forth in SEQ ID NO: 1997. In some instances, the antibody comprises: (i) a VH comprising a VH CDR1 comprising or consisting of the amino acid sequence set forth in SEQ ID NO:48, a VH CDR2 comprising or consisting of the amino acid sequence set forth in SEQ ID NO:137, and a VH CDR3 comprising or consisting of the amino acid sequence set forth in SEQ ID NO:225; and (ii) a VL comprising a VL CDR1 comprising or consisting of the amino acid sequence set forth in SEQ ID NO:301, a VL CDR2 comprising or consisting of the amino acid sequence set forth in SEQ ID NO:376, and a VL CDR3 comprising or consisting of the amino acid sequence set forth in SEQ ID NO:426. In some instances, the antibody comprises: (i) a VH comprising or consisting of the amino acid sequence set forth in SEQ ID NO:1982; and (ii) a VL comprising or consisting of the amino acid sequence set forth in SEQ ID NO:1997. In some instances, the antibody comprises: (i) a heavy chain comprising or consisting of the amino acid sequence set forth in SEQ ID NO:2010; and (ii) a light chain comprising or consisting of the amino acid sequence set forth in SEQ ID NO: 2012. In some instances, the therapeutically effective amount of the antibody is administered as a single dose. In some instances, the single dose is 30 mg, 50 mg, 75 mg, or 100 mg. In certain cases, the single dose is administered subcutaneously. In other cases, the single dose is administered intravenously. In one instance, the single dose is 75 mg and administered subcutaneously. In some instances, the single dose is administered during gestational week 4, 5, 6, 7, 8, 9, 10, 11, 12, 13, 14, 15, 16, 17, 18, 19, 20, 21, 22, 23, 24, 25, 26, 27, 28, 30, 32, 34, or 36.

Also provided herein is a method for reducing nausea and/or vomiting in a human subject in need thereof, the method comprising administering to the human subject a therapeutically effective amount of an antibody that specifically binds human GFRAL. In some instances, the antibody comprises (i) a VH comprising the VH CDR1, the VH CDR2, the VH CDR3 from the amino acid sequence set forth in SEQ ID NO: 1982; and (ii) a VL comprising the VL CDR1, the VL CDR2, and the VL CDR3 from the amino acid sequence set forth in SEQ ID NO: 1997. In some instances, the antibody comprises: (i) a VH comprising a VH CDR1 comprising or consisting of the amino acid sequence set forth in SEQ ID NO:48, a VH CDR2 comprising or consisting of the amino acid sequence set forth in SEQ ID NO:137, and a VH CDR3 comprising or consisting of the amino acid sequence set forth in SEQ ID NO:225; and (ii) a VL comprising a VL CDR1 comprising or consisting of the amino acid sequence set forth in SEQ ID NO:301, a VL CDR2 comprising or consisting of the amino acid sequence set forth in SEQ ID NO:376, and a VL CDR3 comprising or consisting of the amino acid sequence set forth in SEQ ID NO:426. In some instances, the antibody comprises: (i) a VH comprising or consisting of the amino acid sequence set forth in SEQ ID NO:1982; and (ii) a VL comprising or consisting of the amino acid sequence set forth in SEQ ID NO:1997. In some instances, the antibody comprises: (i) a heavy chain comprising or consisting of the amino acid sequence set forth in SEQ ID NO:2010; and (ii) a light chain comprising or consisting of the amino acid sequence set forth in SEQ ID NO: 2012. In some instances, the therapeutically effective amount of the antibody is administered as a single dose. In some instances, the single dose is 30 mg. In some instances, the single dose is 75 mg. In some instances, the single dose is 100 mg. In certain instances, the dose is administered subcutaneously. In other cases, the dose is administered intravenously. In some instances, the single dose is administered during gestational week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36.

Also provided herein is a method for treating HG in a human subject in need thereof, the method comprising administering to the human subject a therapeutically effective amount of an antibody that specifically binds human GFRAL. In some instances, the antibody comprises (i) a VH comprising the VH CDR1, the VH CDR2, the VH CDR3 from the amino acid sequence set forth in SEQ ID NO: 7; and (ii) a VL comprising the VL CDR1, the VL CDR2, and the VL CDR3 from the amino acid sequence set forth in SEQ ID NO: 8. In some instances, the antibody comprises: (i) a VH comprising a VH CDR1 comprising or consisting of the amino acid sequence set forth in SEQ ID NO:58, a VH CDR2 comprising or consisting of the amino acid sequence set forth in SEQ ID NO:147, and a VH CDR3 comprising or consisting of the amino acid sequence set forth in SEQ ID NO:233; and (ii) a VL comprising a VL CDR1 comprising or consisting of the amino acid sequence set forth in SEQ ID NO:309, a VL CDR2 comprising or consisting of the amino acid sequence set forth in SEQ ID NO:382, and a VL CDR3 comprising or consisting of the amino acid sequence set forth in SEQ ID NO:432. In some instances, the antibody comprises: (i) a VH comprising or consisting of the amino acid sequence set forth in SEQ ID NO:7; and (ii) a VL comprising or consisting of the amino acid sequence set forth in SEQ ID NO:8. In some instances, the therapeutically effective amount of the antibody is administered as a single dose. In some instances, the single dose is 30 mg. In some instances, the single dose is 75 mg. In some instances, the single dose is 100 mg. In certain cases, the single dose is administered subcutaneously. In other cases, the single dose is administered intravenously. In some instances, the single dose is administered during gestational week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36. In some instances, the therapeutically effective amount of the antibody is administered as more than one dose (e.g., 1, 2, 3, 4, 5, 6, 7, 8, 9, or 10 doses). In some instances, the first dose is administered during gestational week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36.

Also provided herein is a method for treating NVP in a human subject in need thereof, the method comprising administering to the human subject a therapeutically effective amount of an antibody that specifically binds human GFRAL. In some instances, the antibody comprises (i) a VH comprising the VH CDR1, the VH CDR2, the VH CDR3 from the amino acid sequence set forth in SEQ ID NO: 7; and (ii) a VL comprising the VL CDR1, the VL CDR2, and the VL CDR3 from the amino acid sequence set forth in SEQ ID NO: 8. In some instances, the antibody comprises: (i) a VH comprising or consisting of a VH CDR1 comprising the amino acid sequence set forth in SEQ ID NO:58, a VH CDR2 comprising or consisting of the amino acid sequence set forth in SEQ ID NO: 147, and a VH CDR3 comprising or consisting of the amino acid sequence set forth in SEQ ID NO:233; and (ii) a VL comprising a VL CDR1 comprising or consisting of the amino acid sequence set forth in SEQ ID NO:309, a VL CDR2 comprising or consisting of the amino acid sequence set forth in SEQ ID NO:382, and a VL CDR3 comprising or consisting of the amino acid sequence set forth in SEQ ID NO:432. In some instances, the antibody comprises: (i) a VH comprising or consisting of the amino acid sequence set forth in SEQ ID NO:7; and (ii) a VL comprising or consisting of the amino acid sequence set forth in SEQ ID NO:8. In some instances, the therapeutically effective amount of the antibody is administered as a single dose. In some instances, the single dose is 30 mg. In some instances, the single dose is 75 mg. In some instances, the single dose is 100 mg. In certain cases, the single dose is administered subcutaneously. In other cases, the single dose is administered intravenously. In some instances, the single dose is administered during gestational week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36. In some instances, the therapeutically effective amount of the antibody is administered as more than one dose (e.g., 1, 2, 3, 4, 5, 6, 7, 8, 9, or 10 doses). In some instances, the first dose is administered during gestational week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36.

Also provided herein is a method for treating nausea and/or vomiting in a human subject in need thereof, the method comprising administering to the human subject a therapeutically effective amount of an antibody that specifically binds human GFRAL. In some instances, the antibody comprises (i) a VH comprising the VH CDR1, the VH CDR2, the VH CDR3 from the amino acid sequence set forth in SEQ ID NO: 7; and (ii) a VL comprising the VL CDR1, the VL CDR2, and the VL CDR3 from the amino acid sequence set forth in SEQ ID NO: 8. In some instances, the antibody comprises: (i) a VH comprising a VH CDR1 comprising or consisting of the amino acid sequence set forth in SEQ ID NO:58, a VH CDR2 comprising or consisting of the amino acid sequence set forth in SEQ ID NO: 147, and a VH CDR3 comprising or consisting of the amino acid sequence set forth in SEQ ID NO: 233; and (ii) a VL comprising a VL CDR1 comprising or consisting of the amino acid sequence set forth in SEQ ID NO:309, a VL CDR2 comprising or consisting of the amino acid sequence set forth in SEQ ID NO:382, and a VL CDR3 comprising or consisting of the amino acid sequence set forth in SEQ ID NO:432. In some instances, the antibody comprises: (i) a VH comprising or consisting of the amino acid sequence set forth in SEQ ID NO: 7; and (ii) a VL comprising or consisting of the amino acid sequence set forth in SEQ ID NO: 8. In some instances, the therapeutically effective amount of the antibody is administered as a single dose. In some instances, the single dose is 30 mg. In some instances, the single dose is 75 mg. In some instances, the single dose is 100 mg. In some cases, the single dose is administered subcutaneously. In other cases, the single dose is administered intravenously. In some instances, the single dose is administered during gestational week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36. In some instances, the therapeutically effective amount of the antibody is administered as more than one dose (e.g., 1, 2, 3, 4, 5, 6, 7, 8, 9, or 10 doses). In some instances, the first dose is administered during gestational week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36.

Also provided herein is a method for reducing nausea and/or vomiting in a human subject in need thereof, the method comprising administering to the human subject a therapeutically effective amount of an antibody that specifically binds human GFRAL. In some instances, the antibody comprises (i) a VH comprising the VH CDR1, the VH CDR2, the VH CDR3 from the amino acid sequence set forth in SEQ ID NO: 7; and (ii) a VL comprising the VL CDR1, the VL CDR2, and the VL CDR3 from the amino acid sequence set forth in SEQ ID NO: 8. In some instances, the antibody comprises: (i) a VH comprising a VH CDR1 comprising or consisting of the amino acid sequence set forth in SEQ ID NO:58, a VH CDR2 comprising or consisting of the amino acid sequence set forth in SEQ ID NO:147, and a VH CDR3 comprising or consisting of the amino acid sequence set forth in SEQ ID NO: 233; and (ii) a VL comprising a VL CDR1 comprising or consisting of the amino acid sequence set forth in SEQ ID NO:309, a VL CDR2 comprising or consisting of the amino acid sequence set forth in SEQ ID NO:382, and a VL CDR3 comprising or consisting of the amino acid sequence set forth in SEQ ID NO:432. In some instances, the antibody comprises: (i) a VH comprising or consisting of the amino acid sequence set forth in SEQ ID NO: 7; and (ii) a VL comprising or consisting of the amino acid sequence set forth in SEQ ID NO: 8. In some instances, the therapeutically effective amount of the antibody is administered as a single dose. In some instances, the single dose is 30 mg. In some instances, the single dose is 75 mg. In some instances, the single dose is 100 mg. In some cases, the single dose is administered subcutaneously. In other cases, the single dose is administered intravenously. In some instances, the single dose is administered during gestational week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36. In some instances, the therapeutically effective amount of the antibody is administered as more than one dose (e.g., 1, 2, 3, 4, 5, 6, 7, 8, 9, or 10 doses). In some instances, the first dose is administered during gestational week 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36

In each of the above methods of treatment, the human subject may also be administered a further treatment comprising one or more of: doxylamine (e.g., doxylamine succinate), pyridoxine (e.g., pyridoxine hydrochloride (Vitamin B6)), a dual release formulation of doxylamine and pyridoxine, ondansetron, dopamine antagonists (e.g., metoclopramide, promethazine), diphenhydramine, droperidol, methylprednisolone, mirtazapine, dimenhydrinate, chlorpromazine, prochlorperazine, steroid therapy, an anti-emetic, vitamin B1, folic acid, vitamin K, vitamin D, Magnesium, and ginger. The further treatment may be provided before, at substantially the same time, or after the administration of the antibody that specifically binds human GFRAL.

Also provided is a composition comprising a combination of any of the anti-GFRAL antibodies described herein and a further treatment for HG or NVP. In some instances, the further treatment comprises one or more of: doxylamine (e.g., doxylamine succinate), pyridoxine (e.g., pyridoxine hydrochloride (Vitamin B6)), a dual release formulation of doxylamine and pyridoxine, ondansetron, dopamine antagonists (e.g., metoclopramide, promethazine), diphenhydramine, droperidol, methylprednisolone, mirtazapine, dimenhydrinate, chlorpromazine, prochlorperazine, steroid therapy, an anti-emetic, vitamin B1, folic acid, vitamin K, vitamin D, Magnesium, and ginger.

In another aspect the disclosure features a method for (i) treating hyperemesis gravidarum in a human subject in need thereof, (ii) treating nausea and vomiting of pregnancy (NVP) in a human subject in need thereof, (iii) treating nausea and/or vomiting in a human subject in need thereof, or (iv) reducing nausea and/or vomiting in a human subject in need thereof. The method comprises administering to the human subject a single dose of an antibody that specifically binds human GDNF Family Receptor Alpha-Like (GFRAL), wherein the single dose is about 75 mg or 75 mg and the dose is administered to the subject subcutaneously. In some instances, the anti-GFRAL antibody comprises the six CDRs of the 3P10 antibody (see, Table 1). In some instances, the antibody comprises a VH comprising the sequence set forth in SEQ ID NO: 1982 and a VL comprising the sequence set forth in SEQ ID NO: 1997. In certain instances, the antibody comprises a heavy chain comprising the sequence set forth in SEQ ID NO: 2010 and a light chain comprising the sequence set forth in SEQ ID NO: 2012.

### IV. BRIEF DESCRIPTION OF THE DRAWINGS

FIGS. 1A-1B show alignments of VH and VL sequences of humanized IC1 antibodies. SEQ ID NOS are noted in parenthesis and bold text.

FIGS. 2A-2B show alignments of VH and VL sequences of humanized 25M22 antibodies. SEQ ID NOS are noted in parenthesis and bold text.

FIGS. 3A-3B show alignments of VH and VL sequences of humanized 17J16 antibodies. SEQ ID NOS are noted in parenthesis and bold text.

FIGS. 4A-4B show alignments of VH and VL sequences of humanized 5F12 antibodies. SEQ ID NOS are noted in parenthesis and bold text.

FIGS. 5A-5B show alignments of VH and VL sequences of humanized 3P10 antibodies. SEQ ID NOS are noted in parenthesis and bold text.

FIG. 6 is a graph showing the dose-dependent effects of hz3P10 on body weight gain in mice following the induction of weight loss by a GDF15-Fc fusion protein.

FIG. 7 is a graph showing the dose-dependent weight gain in transgenic GDF15-expressing mice treated with hz3P10.

FIG. 8 is a graph showing the change in body weight in HT1080 tumor-bearing mice treated with hz3P10.

FIG. 9A is a graph showing the serum GDF15 levels in mouse tumor models compared to non-tumor bearing controls. FIG. 9B is a graph showing the serum GDF15 levels in patient-derived xenograft (PDX) mouse tumor models compared to non-tumor bearing controls.

FIGS. 10A-C are graphs showing the body weight changes in the KLN205 (FIG. 10A), Renca (FIG. 10B), and P16b16 (FIG. 10C) syngeneic mouse tumor models following treating with 3P10 or IgG control.

FIGS. 11A-C are graphs showing the body weight changes in gastric (FIG. 11A), ovarian (FIG. 11B), and liver (FIG. 11C) PDX mouse tumor models following treating with hz3P10 or IgG control.

FIGS. 12A-B are graphs showing the serum GDF15 level (FIG. 12A) and body weight change (FIG. 12B) of mice treated with cisplatin alone or in combination with hz3P10, and controls.

FIG. 13 is a graph showing the body weight change over time of rats administered a human GDF15-Fc fusion protein followed two days later with a single dose of hz3P10 or vehicle control.

FIG. 14 is a graph showing the average daily body weight of rats treated weekly with cisplatin and hz3P10 or vehicle controls.

FIG. 15A is a graph showing the simulated pharmacokinetic profile of a 75 kg human administered a single dose of 75 mg hz3P10 (1 mg/kg) over the first seven days along with the actual serum concentrations measured in healthy volunteers administered 10 mg, 30 mg, 100 mg, 200 mg, or 400 mg hz3P10 as a single dose. FIG. 15B is a graph showing the simulated pharmacokinetic profile of a 75 kg human administered a single dose of 75 mg hz3P10 (1 mg/kg) over the 30 weeks along with the actual serum concentrations measured in healthy volunteers administered 10 mg, 30 mg, 100 mg, 200 mg, or 400 mg hz3P10 as a single dose.

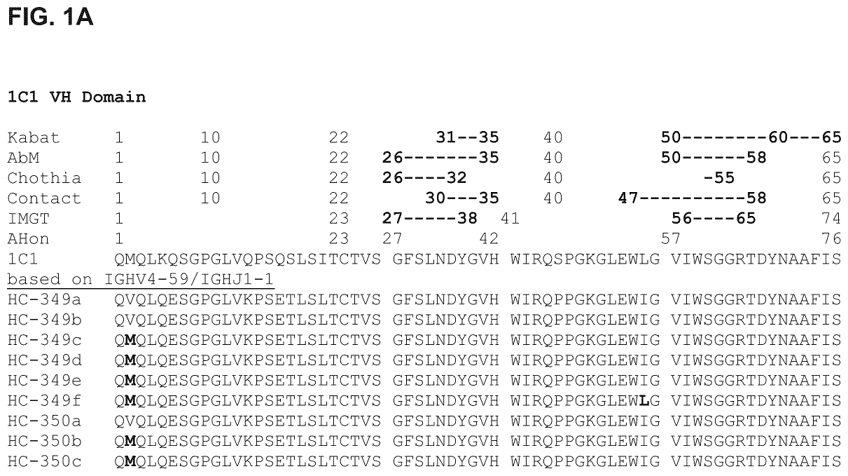

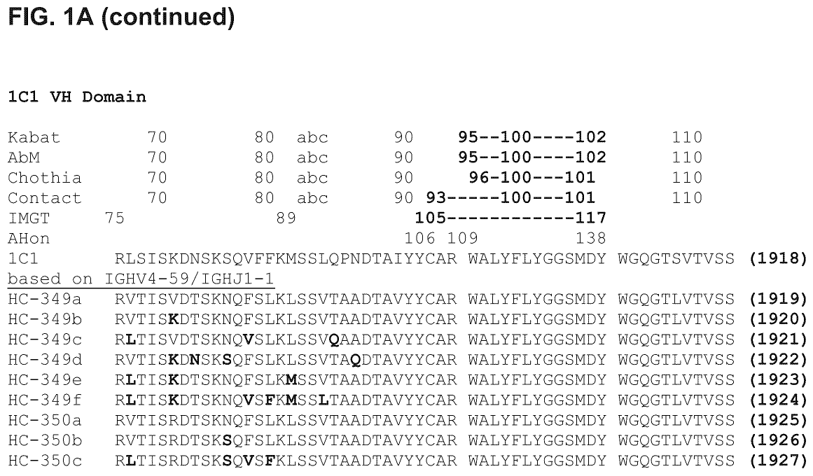

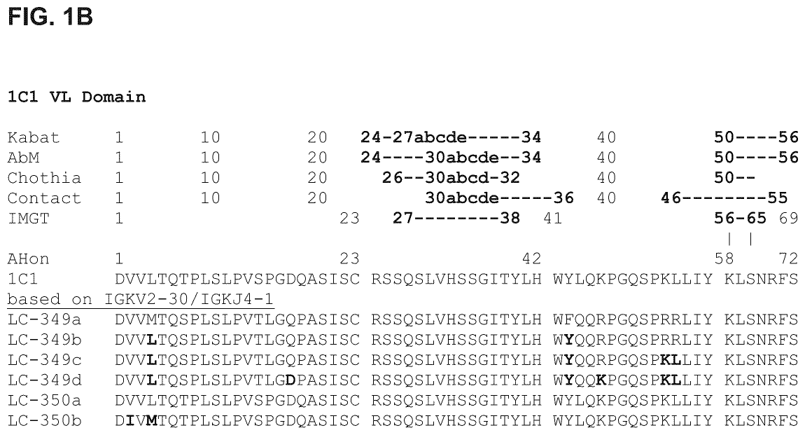

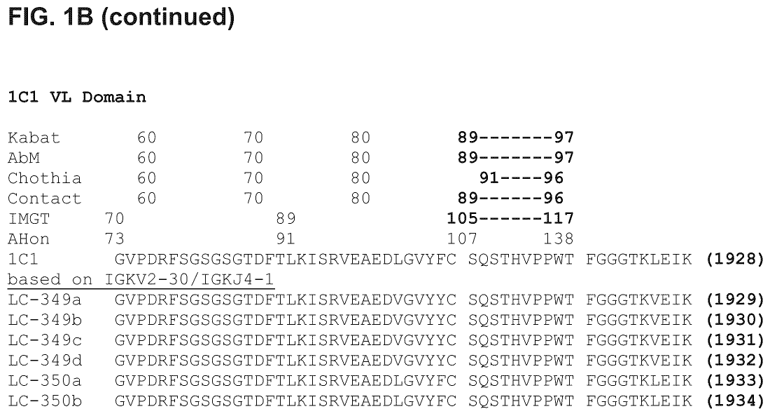

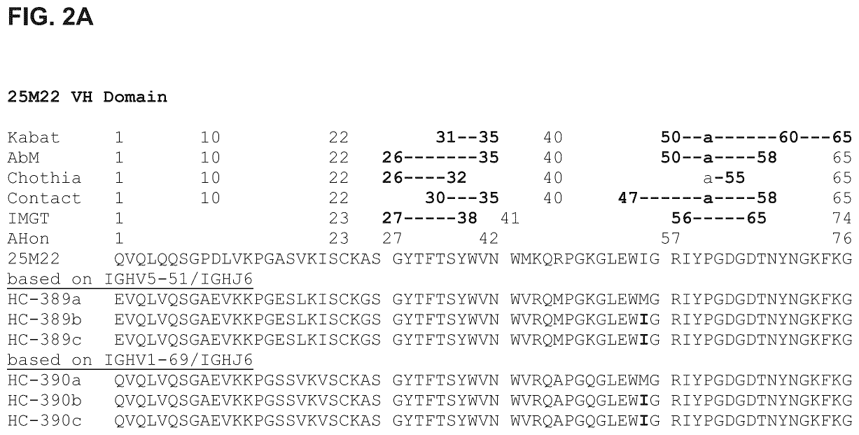

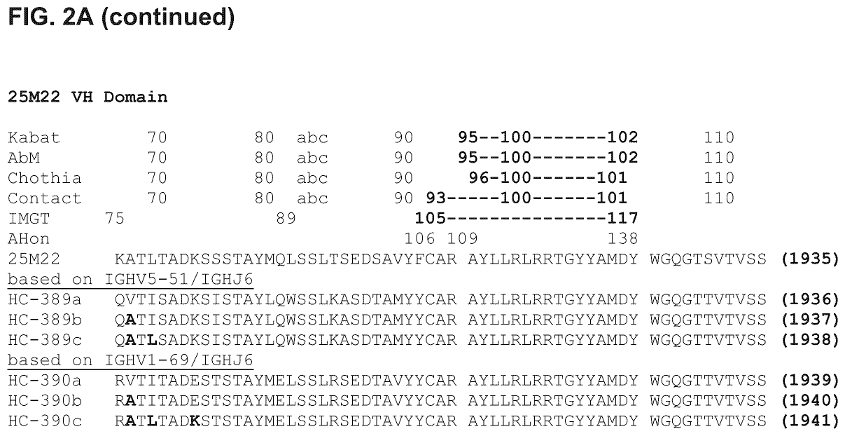

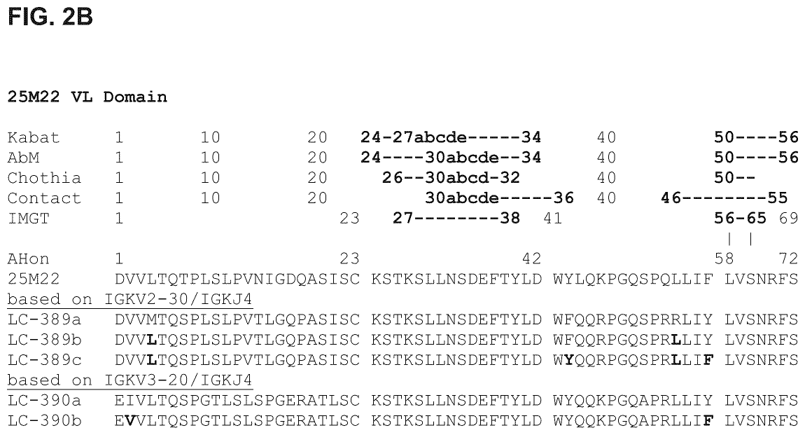

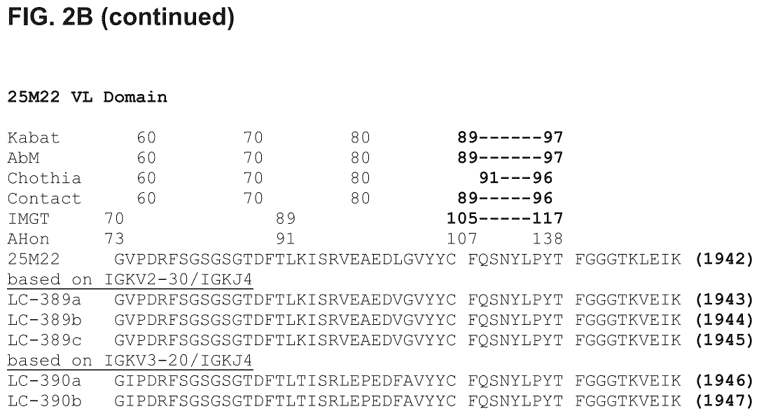

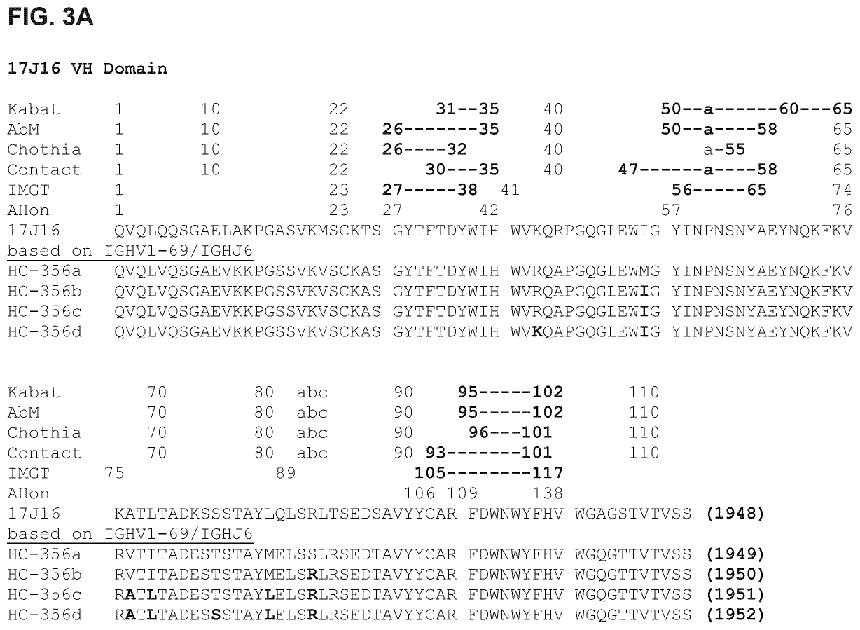

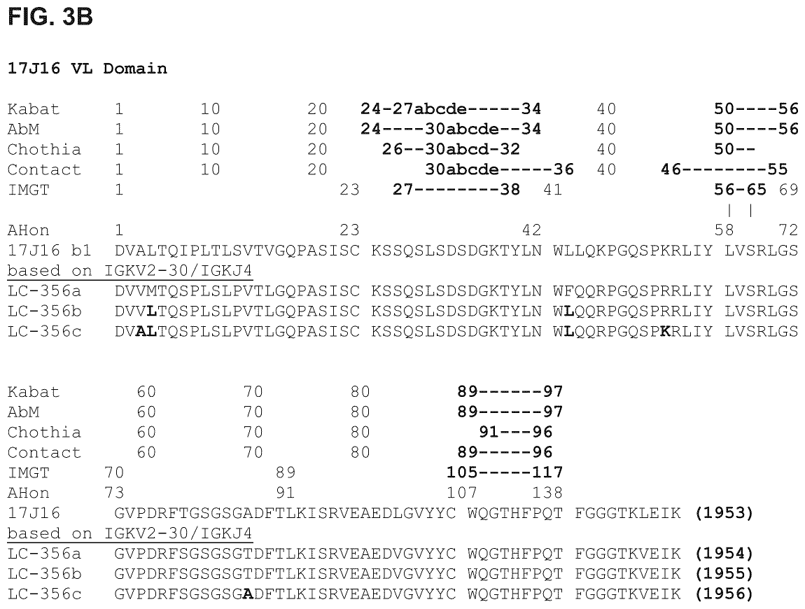

### V. DETAILED DESCRIPTION OF THE INVENTION

The present disclosure relates generally to methods of treating nausea and/or vomiting disorders, such as HG and NVP, in a human subject in need thereof, the method comprising administering to the human subject a therapeutically effective amount of an antibody that specifically binds to human GFRAL.

As used herein throughout, in the context of a dose, “about” means+/−10% of the recited dose. For instance, “about 30 mg” means “27 mg to 33 mg”; “about 75 mg” means “67.5 mg to 82.5 mg”, and “about 100 mg” means “90 mg to 110 mg”. As used herein throughout, in the context of a duration of time, “about” means+/−one week. For instance, “about 3 weeks” means “2 weeks to 4 weeks” and “about 4 weeks” means “3 weeks to 5 weeks”.

Unless explained otherwise, all technical and scientific terms used herein have the same meaning as commonly understood to one of ordinary skill in the art to which this disclosure belongs.

### A. GFRAL, GDF15, and RET

Human GDNF Family Receptor Alpha Like (GFRAL, also known in the art as GDF15 receptor, C6orf144, Chromosome 6 Open reading Frame 144, BA360D14.1, IVF19356, and UNQ9356) is a 375-amino acid (excluding the signal peptide) protein that acts as a receptor for GDF15 signaling.

The amino acid sequence of a full-length precursor human GFRAL is provided below (SEQ ID NO:1797) and includes a signal peptide sequence (lowercase residues):

(SEQ ID NO: 1797)
mivfiflamglsleneytsQTNNCTYLREQCLRDANGCKHAWRVM
EDACNDSDPGDPCKMRNSSYCNLSIQYLVESNFQFKECLCTDDFY
CTVNKLLGKKCINKSDNVKEDKFKWNLTTRSHHGFKGMWSCLEVA
EACVGDVVCNAQLASYLKACSANGNPCDLKQCQAAIRFFYQNIPF
NIAQMLAFCDCAQSDIPCQQSKEALHSKTCAVNMVPPPTCLSVIR
SCQNDELCRRHYRTFQSKCWQRVTRKCHEDENCISTLSKQDLTCS
GSDDCKAAYIDILGTVLQVQCTCRTITQSEESLCKIFQHMLHRKS
CFNYPTLSNVKGMALYTRKHANKITLTGFHSPFNGEVIYAAMCMT
VTCGILLLVMVKLRTSRISSKARDPSSIQIPGEL.

The amino acid sequence of a mature human GFRAL polypeptide is provided below (SEQ ID NO:1798):

(SEQ ID NO: 1798)
QTNNCTYLREQCLRDANGCKHAWRVMEDACNDSDPGDPCKMRNSS
YCNLSIQYLVESNFQFKECLCTDDFYCTVNKLLGKKCINKSDNVK
EDKFKWNLTTRSHHGFKGMWSCLEVAEACVGDVVCNAQLASYLKA
CSANGNPCDLKQCQAAIRFFYQNIPFNIAQMLAFCDCAQSDIPCQ
QSKEALHSKTCAVNMVPPPTCLSVIRSCQNDELCRRHYRTFQSKC
WQRVTRKCHEDENCISTLSKQDLTCSGSDDCKAAYIDILGTVLQV
QCTCRTITQSEESLCKIFQHMLHRKSCFNYPTLSNVKGMALYTRK
HANKITLTGFHSPENGEVIYAAMCMTVTCGILLLVMVKLRTSRIS
SKARDPSSIQIPGEL.

Human GFRAL has an extracellular domain (e.g., residues 20-351 of the amino acid sequence set forth in SEQ ID NO: 1797), a transmembrane domain (e.g., residues 352-371 of the amino acid sequence set forth in SEQ ID NO: 1797) and a cytoplasmic domain (e.g., residues 372-394 of the amino acid sequence set forth in SEQ ID NO: 1797).

GDF15 (also known in the art as MIC-1 (macrophage inhibitory cytokine-1), PDF (prostate differentiation factor), PLAB (placental bone morphogenetic protein), NAG-1 (non-steroidal anti-inflammatory drugs (NSAIDs) activated gene), TGF-PL, and PTGFB), is a member of the transforming growth factor β (TGF-β) super-family. GDF15, which is synthesized as a 62 kDa intracellular precursor protein that is subsequently cleaved by a furin-like protease, is secreted as a 25 kDa disulfide-linked protein (see, e.g., Fairlie et al., J. Leukoc. Biol 65:2-5 (1999)). GDF15 mRNA is seen in several tissues, including liver, kidney, pancreas, colon and placenta, and GDF15 expression in liver can be significantly up-regulated during injury of organs such as the liver, kidneys, heart and lungs. GDF15 also is upregulated during pregnancy and is produced in the placenta.

The GDF15 precursor is a 308 amino acid polypeptide (NCBI Ref. Seq. NP_004855.2; GI: 153792495) containing a 29 amino acid signal peptide, a 167 amino acid pro-domain, and a mature domain of 112 amino acids which is excised from the pro-domain by furin-like proteases.

An amino acid sequence of a precursor human GDF15 polypeptide is provided below (SEQ ID NO:1810):

(SEQ ID NO: 1810)
MPGQELRTVNGSQMLLVLLVLSWLPHGGALSLAEASRASFPGPSE
LHSEDSRFRELRKRYEDLLTRLRANQSWEDSNTDLVPAPAVRILT
PEVRLGSGGHLHLRISRAALPEGLPEASRLHRALFRLSPTASRSW
DVTRPLRRQLSLARPQAPALHLRLSPPPSQSDQLLAESSSARPQL
ELHLRPQAARGRRRARARNGDHCPLGPGRCCRLHTVRASLEDLGW
ADWVLSPREVQVTMCIGACPSQFRAANMHAQIKTSLHRLKPDTVP
APCCVPASYNPMVLIQKTDTGVSLQTYDDLLAKDCHCI.

Such a 308-amino acid GDF15 polypeptide is referred to as a “full-length” GDF15 polypeptide; a 112-amino acid GDF15 polypeptide (amino acids 197-308 of “full-length” GDF15) is a “mature” GDF15 polypeptide. An amino acid sequence of a mature human GDF15 polypeptide is provided below (SEQ ID NO:1811):

(SEQ ID NO: 1811)
ARNGDHCPLGPGRCCRLHTVRASLEDLGWADWVLSPREVQVTMCI
GACPSQFRAANMHAQIKTSLHRLKPDTVPAPCCVPASYNPMVLIQ
KTDTGVSLQTYDDLLAKDCHCI.

“RET” (also known in the art as Ret Proto-Oncogene, Cadherin-Related Family Member 16, Rearranged During Transfection, RET Receptor Tyrosine Kinase, Cadherin Family Member 12, Proto-Oncogene C-Ret, EC 2.7.10.1, CDHF12, CDHR16, RET51, PTC, Hydroxyaryl-Protein Kinase, RET Transforming Sequence, and Receptor Tyrosine Kinase) is one of the receptor tyrosine kinases, cell-surface molecules that transduce signals for cell growth and differentiation. RET acts as a co-receptor and is known as a primary signaling receptor for glial-cell-line-derived neurotrophic factor (GDNF) ligands (in human, GDNF, artemin, neurturin, and persephin) when bound to members of the GDNF receptor alpha (GFRa) co-receptors. A RET protein (e.g., a RET-ECD) comprises 4 consecutive cadherin-like domains (CLD1-CLD4) followed by a membrane proximal cysteine rich domain (CRD). RET protein is a co-receptor with a GFRAL protein and a GDF15 protein (e.g., acting as a co-receptor with a RET protein). A receptor complex includes a GFRAL protein, such as a RET/GFRAL complex, a GFRAL/GDF15 complex, and a RET/GFRAL/GDF15 complex.

RET is distinct from TGFβ RI and TGFβ RII. SEQ ID NO: 1812 is a sequence of a mature human RET9 that lacks a signal peptide:

(SEQ ID NO: 1812)
KVALGLYFSRDAYWEKLYVDQAAGTPLLYVHALRDAPEEVPSFRL
GQHLYGTYRTRLHENNWICIQEDTGLLYLNRSLDHSSWEKLSVRN
RGFPLLTVYLKVFLSPTSLREGECQWPGCARVYFSFFNTSFPACS
SLKPRELCFPETRPSFRIRENRPPGTFHQFRLLPVQFLCPNISVA
YRLLEGEGLPFRCAPDSLEVSTRWALDREQREKYELVAVCTVHAG
AREEVVMVPFPVTVYDEDDSAPTFPAGVDTASAVVEFKRKEDTVV
ATLRVFDADVVPASGELVRRYTSTLLPGDTWAQQTFRVEHWPNET
SVQANGSFVRATVHDYRLVLNRNLSISENRTMQLAVLVNDSDFQG
PGAGVLLLHFNVSVLPVSLHLPSTYSLSVSRRARRFAQIGKVCVE
NCQAFSGINVQYKLHSSGANCSTLGVVTSAEDTSGILFVNDTKAL
RRPKCAELHYMVVATDQQTSRQAQAQLLVTVEGSYVAEEAGCPLS
CAVSKRRLECEECGGLGSPTGRCEWRQGDGKGITRNFSTCSPSTK
TCPDGHCDVVETQDINICPQDCLRGSIVGGHEPGEPRGIKAGYGT
CNCFPEEEKCFCEPEDIQDPLCDELCRTVIAAAVLFSFIVSVLLS
AFCIHCYHKFAHKPPISSAEMTFRRPAQAFPVSYSSSGARRPSLD
SMENQVSVDAFKILEDPKWEFPRKNLVLGKTLGEGEFGKVVKATA
FHLKGRAGYTTVAVKMLKENASPSELRDLLSEFNVLKQVNHPHVI
KLYGACSQDGPLLLIVEYAKYGSLRGFLRESRKVGPGYLGSGGSR
NSSSLDHPDERALTMGDLISFAWQISQGMQYLAEMKLVHRDLAAR
NILVAEGRKMKISDFGLSRDVYEEDSYVKRSQGRIPVKWMAIESL
FDHIYTTQSDVWSFGVLLWEIVTLGGNPYPGIPPERLFNLLKTGH
RMERPDNCSEEMYRLMLQCWKQEPDKRPVFADISKDLEKMMVKRR
DYLDLAASTPSDSLIYDDGLSEEETPLVDCNNAPLPRALPSTWIE
NKLYGRISHAFTRF

The amino acid sequence of a full-length precursor human RET protein is provided below (SEQ ID NO:1813) and includes a signal peptide sequence (lowercase residues):

(SEQ ID NO: 1813)
makatsgaaglrlllllllpllgkvalgLYFSRDAYWEKLYVDQA
AGTPLLYVHALRDAPEEVPSFRLGQHLYGTYRTRLHENNWICIQE
DTGLLYLNRSLDHSSWEKLSVRNRGFPLLTVYLKVFLSPTSLREG
ECQWPGCARVYFSFFNTSFPACSSLKPRELCFPETRPSFRIRENR
PPGTFHQFRLLPVQFLCPNISVAYRLLEGEGLPFRCAPDSLEVST
RWALDREQREKYELVAVCTVHAGAREEVVMVPFPVTVYDEDDSAP
TFPAGVDTASAVVEFKRKEDTVVATLRVFDADVVPASGELVRRYT
STLLPGDTWAQQTFRVEHWPNETSVQANGSFVRATVHDYRLVLNR
NLSISENRTMQLAVLVNDSDFQGPGAGVLLLHFNVSVLPVSLHLP
STYSLSVSRRARRFAQIGKVCVENCQAFSGINVQYKLHSSGANCS
TLGVVTSAEDTSGILFVNDTKALRRPKCAELHYMVVATDQQTSRQ
AQAQLLVTVEGSYVAEEAGCPLSCAVSKRRLECEECGGLGSPTGR
CEWRQGDGKGITRNFSTCSPSTKTCPDGHCDVVETQDINICPQDC
LRGSIVGGHEPGEPRGIKAGYGTCNCFPEEEKCFCEPEDIQDPLC
DELCRTVIAAAVLFSFIVSVLLSAFCIHCYHKFAHKPPISSAEMT
FRRPAQAFPVSYSSSGARRPSLDSMENQVSVDAFKILEDPKWEFP
RKNLVLGKTLGEGEFGKVVKATAFHLKGRAGYTTVAVKMLKENAS
PSELRDLLSEFNVLKQVNHPHVIKLYGACSQDGPLLLIVEYAKYG
SLRGFLRESRKVGPGYLGSGGSRNSSSLDHPDERALTMGDLISFA
WQISQGMQYLAEMKLVHRDLAARNILVAEGRKMKISDFGLSRDVY
EEDSYVKRSQGRIPVKWMAIESLFDHIYTTQSDVWSFGVLLWEIV
TLGGNPYPGIPPERLFNLLKTGHRMERPDNCSEEMYRLMLQCWKQ
EPDKRPVFADISKDLEKMMVKRRDYLDLAASTPSDSLIYDDGLSE
EETPLVDCNNAPLPRALPSTWIENKLYGRISHAFTRF

B. Antibodies that Bind to Human GFRAL

The present disclosure provides antibodies that bind to human GFRAL (also referred to herein as “anti-GFRAL antibodies”) and may be used in the methods of treating described herein. Antibodies described herein include those known in the art, such as those described in International Patent Application Publication Nos. WO 2017/172260 and WO 2022/207785, each of which is incorporated by reference herein in its entirety.

Antibodies described herein may antagonize an effect of a GDF15 protein, including block the formation of a GDF15/GFRAL protein complex or a GDF15/GFRAL/RET protein complex or block GDF15 signaling, including, for example, as measured by several in vitro cell-based assays. Such assays may include (1) an ELK1-luciferase reporter assay (see, e.g., Example 3 of International Patent Application Publication No. WO 2017/172260, which is incorporated by reference herein in its entirety); and/or (2) am ERK-phosphorylation assay in U2OS cells (see, e.g., Example 4 of International Patent Application Publication No. WO 2017/172260, which is incorporated by reference herein in its entirety). The antibodies described herein, therefore, are expected to inhibit GDF15 activities in vivo (e.g., related to the signaling function of GDF15). This property makes the disclosed antibodies viable therapeutics for the treatment of a disease, disorder or condition that is caused by or otherwise associated with a GDF15 protein (e.g., a human GDF15 protein) and/or a GFRAL protein (e.g., a human GFRAL protein), such as those related to GDF15-induced signaling in a subject.

In some instances, an antibody described herein inhibits human GFRAL binding to human RET. Methods of determining whether an antibody inhibits human GFRAL binding to human RET are known in the art, such as, e.g., co-immunoprecipitation, immunohistochemistry, and ELISA (see, e.g., International Patent Application Publication No. WO 2017/172260, which is incorporated by reference herein in its entirety). Exemplary antibodies that inhibit human GFRAL binding to human RET include Hz3P10, 3P10, 5F12, 2123, 6N16, 1B3, 6G9, or 2B11 (see, e.g., the working examples of International Patent Application Publication No. WO 2017/172260, which is incorporated by reference herein in its entirety).

In some instances, an antibody described herein does not inhibit human GFRAL binding to human GDF15. Methods of determining whether an antibody does not inhibit human GFRAL binding to human GDF15 are known in the art, such as, e.g., co-immunoprecipitation, immunohistochemistry, and ELISA (see, e.g., International Patent Application Publication No. WO 2017/172260, which is incorporated by reference herein in its entirety). Exemplary antibodies that do not inhibit human GFRAL binding to human GDF15 include 5F12, 3P10, Hz3P10, 6N16, 2B11, 1B3, 2123, 1A3, P1B6, P1H8, and P8G4 (see, e.g., the working examples of International Patent Application Publication No. WO 2017/172260, which is incorporated by reference herein in its entirety). In some instances, an antibody described herein (i) inhibits human GFRAL binding to human RET and (ii) does not inhibit human GFRAL binding to human GDF15.

In some instances, an antibody described herein inhibits human GFRAL binding to human GDF15. Methods of determining whether an antibody inhibits human GFRAL binding to human GDF15 are known in the art, such as, e.g., co-immunoprecipitation, immunohistochemistry, and ELISA (see, e.g., International Patent Application Publication No. WO 2017/172260, which is incorporated by reference herein in its entirety). Exemplary antibodies that inhibit human GFRAL binding to human GDF15 include 2B8, 8C10, 25M22, 12A3, 19K19, 1C1, 8D8, 22N5, 2A9, 2B3, 24G2, 17J16, and 5A20 (see, e.g., the working examples of International Patent Application Publication No. WO 2017/172260, which is incorporated by reference herein in its entirety).

In some instances, the antibody comprises a VH comprising a VH CDR1, a VH CDR2, a VH CDR3, and a VL comprising a VL CDR1, a VL CDR2, and a VL CDR3, wherein the VH CDR1, VH CDR2, VH CDR3, VL CDR1, VL CDR2 and VL CDR3 are from any one of the VH and VL sequences of an antibody described herein, such as the VH and VL amino acid sequences depicted in Tables 1-24. In some instances, the antibody is a humanized version of an antibody described herein. In some instances, a humanized version of an antibody described herein comprises a VH comprising a VH CDR1, a VH CDR2, a VH CDR3, and a VL comprising a VL CDR1, a VL CDR2, and a VL CDR3, wherein the VH CDR1, VH CDR2, and VH CDR3 are from the amino acid sequence of the VH of the antibody described herein, and the VL CDR1, VL CDR2 and VL CDR3 are from the amino acid sequence of the VL of the antibody described herein (see Tables 1-24).

The antibody designated 3P10 comprises a VH comprising the sequence set forth in SEQ ID NO: 3 and a VL comprising the sequence set forth in SEQ ID NO: 4 (see Table 1). Exemplary humanized versions of antibody 3P10 comprise a VH comprising the sequence set forth in any one of SEQ ID NOs: 1978-1988 and a VL comprising the sequence set forth in any one of SEQ ID NOs: 1990-2000.

The antibody designated Hz3P10 comprises a VH comprising the sequence set forth in SEQ ID NO: 1982 and a VL comprising the sequence set forth in SEQ ID NO: 1997 (see Table 1A). The antibody designated Hz3P10 comprises a heavy chain comprising the sequence set forth in SEQ ID NO: 2010 and a light chain comprising the sequence set forth in SEQ ID NO: 2012 (see Table 1A).

The antibody designated 5F12 comprises a comprising the sequence set forth in SEQ ID NO: 7 and a VL comprising the sequence set forth in SEQ ID NO: 8 (see Table 2).

The antibody designated 2123 comprises a VH comprising the sequence set forth in SEQ ID NO: 21 and a VL comprising the sequence set forth in SEQ ID NO: 22 (see Table 3).

The antibody designated 6N16 comprises a VH comprising the sequence set forth in SEQ ID NO: 23 and a VL comprising the sequence set forth in SEQ ID NO: 24 (see Table 4).

The antibody designated 1B3 comprises a VH comprising the sequence set forth in SEQ ID NO: 25 and a VL comprising the sequence set forth in SEQ ID NO: 26 (see Table 5).

The antibody designated 6G9 comprises a VH comprising the sequence set forth in SEQ ID NO: 37 and a VL comprising the sequence set forth in SEQ ID NO: 38 (see Table 6).

The antibody designated 2B11 comprises a VH comprising the sequence set forth in SEQ ID NO: 39 and a VL comprising the sequence set forth in SEQ ID NO: 40 (see Table 7).

The antibody designated 17J16 comprises a VH comprising the sequence set forth in SEQ ID NO: 13 and a VL comprising the sequence set forth in SEQ ID NO: 14 (see Table 8).

The antibody designated 8D8 comprises a VH comprising the sequence set forth in SEQ ID NO: 11 and a VL comprising the sequence set forth in SEQ ID NO: 12 (see Table 9).

The antibody designated 2B3 comprises a VH comprising the sequence set forth in SEQ ID NO: 29 and a VL comprising the sequence set forth in SEQ ID NO: 30 (see Table 10).

The antibody designated 24G2 comprises a VH comprising the sequence set forth in SEQ ID NO: 35 and a VL comprising the sequence set forth in SEQ ID NO: 36 (see Table 11)

The antibody designated 5A20 comprises a VH comprising the sequence set forth in SEQ ID NO: 9 and a VL comprising the sequence set forth in SEQ ID NO: 10 (see Table 12)

The antibody designated 2B8 comprises a VH comprising the sequence set forth in SEQ ID NO: 17 and a VL comprising the sequence set forth in SEQ ID NO: 18 (see Table 13).

The antibody designated 8C10 comprises a VH comprising the sequence set forth in SEQ ID NO: 31 and a VL comprising the sequence set forth in SEQ ID NO: 32 (see Table 14)

The antibody designated 25M22 comprises a VH comprising the sequence set forth in SEQ ID NO: 15 and a VL comprising the sequence set forth in SEQ ID NO: 16 (see Table 15).

The antibody designated 12A3 comprises a VH comprising the sequence set forth in SEQ ID NO: 5 and a VL comprising the sequence set forth in SEQ ID NO: 6 (see Table 16)

The antibody designated 19K19 comprises a VH comprising the sequence set forth in SEQ ID NO: 27 and a VL comprising the sequence set forth in SEQ ID NO: 28 (see Table 17).

The antibody designated 1C1 comprises a VH comprising the sequence set forth in SEQ ID NO: 1 and a VL comprising the sequence set forth in SEQ ID NO: 2 (see Table 18).

The antibody designated 22N5 comprises a VH comprising the sequence set forth in SEQ ID NO: 19 and a VL comprising the sequence set forth in SEQ ID NO: 20 (see Table 19)

The antibody designated 2A9 comprises a VH comprising the sequence set forth in SEQ ID NO: 33 and a VL comprising the sequence set forth in SEQ ID NO: 34 (see Table 20).

The antibody designated 1A3 comprises a VH comprising the sequence set forth in SEQ ID NO: 480 and a VL comprising the sequence set forth in SEQ ID NO: 481 (see Table 21).

The antibody designated P1B6 comprises a VH comprising the sequence set forth in SEQ ID NO: 482 and a VL comprising the sequence set forth in SEQ ID NO: 483 (see Table 22).

The antibody designated P8G4 comprises a VH comprising the sequence set forth in SEQ ID NO: 486 and a VL comprising the sequence set forth in SEQ ID NO: 487 (see Table 23).

The antibody designated P1H8 comprises a VH comprising the sequence set forth in SEQ ID NO: 484 and a VL comprising the sequence set forth in SEQ ID NO: 485 (see Table 24).

TABLE 1
Antibody 3P10 CDR Sequences

| | | Exemplary | IMGT | Kabat |
|---|---|---|---|---|
| VH CDR | VH CDR1 | GYTFTDYGVI | GYTFTDYG | DYGVI |
| Seq. | | (SEQ ID NO: 46) | (SEQ ID NO: 47) | (SEQ ID NO: 48) |
| | VH CDR2 | WINTYTGEPTYAD | INTYTGEP | WINTYTGEPTYAD |
| | | DLKG | (SEQ ID NO: 138) | DLKG |
| | | (SEQ ID NO: 137) | | (SEQ ID NO: 137) |
| | VH CDR3 | RYGPEDIDY | ARRYGPEDIDY | RYGPEDIDY |
| | | (SEQ ID NO: 225) | (SEQ ID NO: 226) | (SEQ ID NO: 225) |
| VL CDR | VL CDR1 | RASESVDNYGISF | ESVDNYGISF | RASESVDNYGISF |
| Seq. | | MS | (SEQ ID NO: 302) | MS |
| | | (SEQ ID NO: 301) | | (SEQ ID NO: 301) |
| | VL CDR2 | AASHQGS | AAS | AASHQGS |
| | | (SEQ ID NO: 376) | | (SEQ ID NO: 376) |
| | VL CDR3 | LQSKEVPWT | LQSKEVPWT | LQSKEVPWT |
| | | (SEQ ID NO: 426) | (SEQ ID NO: 426) | (SEQ ID NO: 426) |
| | VH CDR1 | Chothia | Contact | AbM |
| VH CDR | VH CDR2 | GYTFTDY | TDYGVI | GYTFTDYGVI |
| Seq. | | (SEQ ID NO: 49) | (SEQ ID NO: 50) | (SEQ ID NO: 46) |
| | VH CDR3 | TYTG | WMGWINTYTGEP | WINTYTGEPT |
| | | (SEQ ID NO: 139) | T | (SEQ ID NO: 141) |
| | | | (SEQ ID NO: 140) | |
| VL CDR | VL CDR1 | YGPEDID | ARRYGPEDID | RYGPEDIDY |
| Seq. | | (SEQ ID NO: 227) | (SEQ ID NO: 228) | (SEQ ID NO: 225) |
| | VL CDR2 | SESVDNYGISF | DNYGISFMSWF | RASESVDNYGISF |
| | | (SEQ ID NO: 303) | (SEQ ID NO: 304) | MS |
| | | | | (SEQ ID NO: 301) |
| | VL CDR3 | AAS | LLIYAASHQG | AASHQGS |
| | | | (SEQ ID NO: 378) | (SEQ ID NO: 376) |
| | VH CDR1 | SKEVPW | LQSKEVPW | LQSKEVPWT |
| | | (SEQ ID NO: 427) | (SEQ ID NO: 428) | (SEQ ID NO: 426) |

VH Sequence:
QIQLVQSGPELKKPGETVKISCKASGYTFTDYGVIWVKQAPGKALKWMGWINTYTGEPTYADDLKGRFAFSLETSASSAS
LQINNLKNEDTATYFCARRYGPEDIDYWGQGTTLTVSS(SEQ ID NO: 3)
VL Sequence:
DIVLTQSPVSLAVSLGQRATISCRASESVDNYGISFMSWFQQKPGQPPKLLIYAASHQGSGVPARFSGSGSGTDFSLNIH
PMEEDDSAMYFCLQSKEVPWTFGGGTKLEIK
(SEQ ID NO: 4)

TABLE 1A
Hz3P10 VH, VL, heavy chain (HC), and
light chain (LC) Sequences

| | |
|---|---|
| VH | QVQLVQSGAEVKKPGSSVKVSCKASGYTFTDYGV |
| | IWVRQAPGQGLEWMGWINTYTGEPTYADDLKGRV |
| | TFTADESTSTAYMELSSLRSEDTAVYYCARRYGP |
| | EDIDYWGQGTTVTVSS (SEQ ID NO: 1982) |
| VL | DVVLTQSPLSLPVTLGQPASISCRASESVDNYGI |
| | SFMSWFQQRPGQSPRLLIYAASHQGSGVPDRFSG |
| | SGSGTDFTLKISRVEAEDVGVYFCLQSKEVPWTF |
| | GGGTKVEIK (SEQ ID NO: 1997) |
| HC | MDMRVPAQLLGLLLLWLRGARCQVQLVQSGAEVK |
| (with | KPGSSVKVSCKASGYTFTDYGVIWVRQAPGQGLE |
| signal | WMGWINTYTGEPTYADDLKGRVTFTADESTSTAY |
| sequence | MELSSLRSEDTAVYYCARRYGPEDIDYWGQGTTV |
| in bold; | TVSSASTKGPSVFPLAPSSKSTSGGTAALGCLVK |
| VH + | DYFPEPVTVSWNSGALTSGVHTFPAVLQSSGLYS |
| heavy | LSSVVTVPSSSLGTQTYICNVNHKPSNTKVDKKV |
| chain | EPKSCDKTHTCPPCPAPALAGGPSVFLFPPKPKD |
| constant | TLMISRTPEVTCVVVDVSHEDPEVKFNWYVDGVE |
| region) | VHNAKTKPREEQYNSTYRVVSVLTVLHQDWLNGK |
| | EYKCKVSNKALPAPIEKTISKAKGQPREPQVYTL |
| | PPSREEMTKNQVSLTCLVKGFYPSDIAVEWESNG |
| | QPENNYKTTPPVLDSDGSFFLYSKLTVDKSRWQQ |
| | GNVFSCSVMHEALHNHYTQKSLSLSPGK |
| | (SEQ ID NO: 2009) |
| HC | QVQLVQSGAEVKKPGSSVKVSCKASGYTFTDYGV |
| (without | IWVRQAPGQGLEWMGWINTYTGEPTYADDLKGRV |
| signal | TFTADESTSTAYMELSSLRSEDTAVYYCARRYGP |
| sequence; | EDIDYWGQGTTVTVSSASTKGPSVFPLAPSSKST |
| VH + | SGGTAALGCLVKDYFPEPVTVSWNSGALTSGVHT |
| heavy | FPAVLQSSGLYSLSSVVTVPSSSLGTQTYICNVN |
| chain | HKPSNTKVDKKVEPKSCDKTHTCPPCPAPALAGG |
| constant | PSVFLFPPKPKDTLMISRTPEVTCVVVDVSHEDP |
| region) | EVKFNWYVDGVEVHNAKTKPREEQYNSTYRVVSV |
| | LTVLHQDWLNGKEYKCKVSNKALPAPIEKTISKA |
| | KGQPREPQVYTLPPSREEMTKNQVSLTCLVKGFY |
| | PSDIAVEWESNGQPENNYKTTPPVLDSDGSFFLY |
| | SKLTVDKSRWQQGNVFSCSVMHEALHNHYTQKSL |
| | SLSPGK (SEQ ID NO: 2010) |
| LC | MDMRVPAQLLGLLLLWLRGARCDVVLTQSPLSLP |
| (with | VTLGQPASISCRASESVDNYGISFMSWFQQRPGQ |
| signal | SPRLLIYAASHQGSGVPDRFSGSGSGTDFTLKIS |
| sequence | RVEAEDVGVYFCLQSKEVPWTFGGGTKVEIKRTV |
| in bold; | AAPSVFIFPPSDEQLKSGTASVVCLLNNFYPREA |
| VL + | KVQWKVDNALQSGNSQESVTEQDSKDSTYSLSST |
| kappa | LTLSKADYEKHKVYACEVTHQGLSSPVTKSFNRG |
| light | EC (SEQ ID NO: 2011) |
| chain | |
| constant | |
| region) | |
| LC | DVVLTQSPLSLPVTLGQPASISCRASESVDNYGI |
| (without | SFMSWFQQRPGQSPRLLIYAASHQGSGVPDRFSG |
| signal | SGSGTDFTLKISRVEAEDVGVYFCLQSKEVPWTF |
| sequence; | GGGTKVEIKRTVAAPSVFIFPPSDEQLKSGTASV |
| VL + | VCLLNNFYPREAKVQWKVDNALQSGNSQESVTEQ |
| kappa | DSKDSTYSLSSTLTLSKADYEKHKVYACEVTHQG |
| light | LSSPVTKSFNRGEC (SEQ ID NO: 2012) |
| chain | |
| constant | |
| region) | |

TABLE 2
Antibody 5F12 CDR Sequences

| | | Exemplary | IMGT | Kabat |
|---|---|---|---|---|
| VH CDR | VH CDR1 | GYTFTDYYIN | GYTFTDYY | DYYIN |
| Seq. | | (SEQ ID NO: 56) | (SEQ ID NO: 57) | (SEQ ID NO: 58) |
| | VH CDR2 | RIYPGNGNTYHNE | IYPGNGNT | RIYPGNGNTYHNE |
| | | KFKG | (SEQ ID NO: 148) | KFKG |
| | | (SEQ ID NO: 147) | | (SEQ ID NO: 147) |
| | VH CDR3 | EGLYYDYDRYFD | AREGLYYDYDRY | EGLYYDYDRYFD |
| | | Y | FDY | Y |
| | | (SEQ ID NO: 233) | (SEQ ID NO: 234) | (SEQ ID NO: 233) |
| VL CDR | VL CDR1 | RASESVDTYGNSF | ESVDTYGNSF | RASESVDTYGNSF |
| Seq. | | MH | (SEQ ID NO: 310) | MH |
| | | (SEQ ID NO: 309) | | (SEQ ID NO: 309) |
| | VL CDR2 | LASNLES | LAS | LASNLES |
| | | (SEQ ID NO: 382) | | (SEQ ID NO: 382) |
| | VL CDR3 | HQNNEDPPA | HQNNEDPPA | HQNNEDPPA |
| | | (SEQ ID NO: 432) | (SEQ ID NO: 432) | (SEQ ID NO: 432) |
| | | Chothia | Contact | AbM |
| VH CDR | VH CDR1 | GYTFTDY | TDYYIN | GYTFTDYYIN |
| Seq. | | (SEQ ID NO: 49) | (SEQ ID NO: 59) | (SEQ ID NO: 56) |
| | VH CDR2 | PGNG | WIARIYPGNGNTY | RIYPGNGNTY |
| | | (SEQ ID NO: 149) | (SEQ ID NO: 150) | (SEQ ID NO: 151) |
| | VH CDR3 | GLYYDYDRYFD | AREGLYYDYDRY | EGLYYDYDRYFD |
| | | (SEQ ID NO: 235) | FL | Y |
| | | | (SEQ ID NO: 236) | (SEQ ID NO: 233) |
| VL CDR | VL CDR1 | SESVDTYGNSF | DTYGNSFMHWY | RASESVDTYGNSF |
| Seq. | | (SEQ ID NO: 311) | (SEQ ID NO: 312) | MH |
| | | | | (SEQ ID NO: 309) |
| | VL CDR2 | LAS | LLIYLASNLE | LASNLES |
| | | | (SEQ ID NO: 384) | (SEQ ID NO: 382) |
| | VL CDR3 | NNEDPP | HQNNEDPP | HONNEDPPA |
| | | (SEQ ID NO: 433) | (SEQ ID NO: 434) | (SEQ ID NO: 432) |

VH Sequence:
QVQLKQSGTELVRPGASVKLSCKASGYTFTDYYINWVKQRPGQGLEWIARIYPGNGNTYHNEKFKGKATLTAEKSSSTAYM
QLSSLTSEDSAVYFCAREGLYYDYDRYFDYWGQGTALTVSS (SEQ ID NO: 7)
VL Sequence:
NIVLTQSPASLAVSLGQRATISCRASESVDTYGNSFMHWYQQKPGQPPKLLIYLASNLESGVPARFSGSGSRTDFTLTIDP
VEADDAATYYCHQNNEDPPAFGGGTKLEIK (SEQ ID NO: 8)

TABLE 3
Antibody 2123 CDR Sequences

| | | Exemplary | IMGT | Kabat |
|---|---|---|---|---|
| VH CDR | VH CDR1 | GYSFTSYNID | GYSFTSYN | SYNID |
| Seq. | | (SEQ ID NO: 86) | (SEQ ID NO: 87) | (SEQ ID NO: 88) |
| | VH CDR2 | WIFPGDGST | IFPGDGST | WIFPGDGSTKYNE |
| | | (SEQ ID NO: 177) | (SEQ ID NO: 178) | KFKG |
| | | | | (SEQ ID NO: 179) |
| | VH CDR3 | SGIYYGSHFVY | ARSGIYYGSHFVY | SGIYYGSHFVY |
| | | (SEQ ID NO: 261) | (SEQ ID NO: 262) | (SEQ ID NO: 261) |
| VL CDR | VL CDR1 | RSSQSLLDSDGKT | QSLLDSDGKTY | RSSQSLLDSDGKT |
| Seq. | | YLN | (SEQ ID NO: 338) | YLN |
| | | (SEQ ID NO: 337) | | (SEQ ID NO: 337) |
| | VL CDR2 | LVSKVDS | LVS | LVSKVDS |
| | | (SEQ ID NO: 401) | | (SEQ ID NO: 401) |
| | VL CDR3 | WQGTHFPLT | WQGTHFPLT | WQGTHFPLT |
| | | (SEQ ID NO: 453) | (SEQ ID NO: 453) | (SEQ ID NO: 453) |
| | | Chothia | Contact | AbM |
| VH CDR | VH CDR1 | GYSFTSY | TSYNID | GYSFTSYNID |
| Seq. | | (SEQ ID NO: 89) | (SEQ ID NO: 90) | (SEQ ID NO: 86) |
| | VH CDR2 | PGDG | WIGWIFPGDGSTK | WIFPGDGSTK |
| | | (SEQ ID NO: 168) | (SEQ ID NO: 180) | (SEQ ID NO: 181) |
| | VH CDR3 | GIYYGSHFV | ARSGIYYGSHFV | SGIYYGSHFVY |
| | | (SEQ ID NO: 263) | (SEQ ID NO: 264) | (SEQ ID NO: 261) |
| VL CDR | VL CDR1 | SQSLLDSDGKTY | LDSDGKTYLNWL | RSSQSLLDSDGKT |
| Seq. | | (SEQ ID NO: 339) | (SEQ ID NO: 340) | YLN |
| | | | | (SEQ ID NO: 337) |
| | VL CDR2 | LVS | RLIYLVSKVD | LVSKVDS |
| | | | (SEQ ID NO: 402) | (SEQ ID NO: 401) |
| | VL CDR3 | GTHFPL | WQGTHFPL | WQGTHFPLT |
| | | (SEQ ID NO: 454) | (SEQ ID NO: 455) | (SEQ ID NO: 453) |

VH Sequence:
QAQLQQSGAELVKPGASVKLSCKASGYSFTSYNIDWVRQRPEQGLEWIGWIFPGDGSTKYNEKFKGQATLTTDKSSSTTY
IHLSRLTSEDSAVYFCARSGIYYGSHFVYWGQGTLVTVSA
(SEQ ID NO: 21)
VL Sequence:
DVVMTQTPLTLSVTIGQSASISCRSSQSLLDSDGKTYLNWLLQRPGQSPKRLIYLVSKVDSGVPDRFTGSGSGTDFTLKI
SRVEAEDLGVYFCWQGTHFPLTFGAGTKLELK
(SEQ ID NO: 22)

TABLE 4
Antibody 6N16 CDR Sequences

| | | Exemplary | IMGT | Kabat |
|---|---|---|---|---|
| VH CDR | VH CDR1 | GYTFTSYNIN | GYTFTSYN | SYNIN |
| Seq. | | (SEQ ID NO: 91) | (SEQ ID NO: 92) | (SEQ ID NO: 93) |
| | VH CDR2 | WIFPGDDSIKYNE | IFPGDDSI | WIFPGDDSIKYNE |
| | | NFRG | (SEQ ID NO: 183) | NFRG |
| | | (SEQ ID NO: 182) | | (SEQ ID NO: 182) |
| | VH CDR3 | SGIFYGNNFAY | ARSGIFYGNNFAY | SGIFYGNNFAY |
| | | (SEQ ID NO: 265) | (SEQ ID NO: 266) | (SEQ ID NO: 265) |
| VL CDR | VL CDR1 | KSSQSLLDGDGET | QSLLDGDGETY | KSSQSLLDGDGET |
| Seq. | | YLS | (SEQ ID NO: 342) | YLS |
| | | (SEQ ID NO: 341) | | (SEQ ID NO: 341) |
| | VL CDR2 | LVSKLDS | LVS | LVSKLDS |
| | | (SEQ ID NO: 385) | | (SEQ ID NO: 385) |
| | VL CDR3 | CQSTHFPLT | CQSTHFPLT | CQSTHFPLT |
| | | (SEQ ID NO: 456) | (SEQ ID NO: 456) | (SEQ ID NO: 456) |
| | | Chothia | Contact | AbM |
| VH CDR | VH CDR1 | GYTFTSY | TSYNIN | GYTFTSYNIN |
| Seq. | | (SEQ ID NO: 75) | (SEQ ID NO: 94) | (SEQ ID NO: 91) |
| | VH CDR2 | PGDD | WIGWIFPGDDSIK | WIFPGDDSIK |
| | | (SEQ ID NO: 184) | (SEQ ID NO: 185) | (SEQ ID NO: 186) |
| | VH CDR3 | GIFYGNNFA | ARSGIFYGNNFA | SGIFYGNNFAY |
| | | (SEQ ID NO: 267) | (SEQ ID NO: 268) | (SEQ ID NO: 265) |
| VL CDR | VL CDR1 | SQSLLDGDGETY | LDGDGETYLSWL | KSSQSLLDGDGET |
| Seq. | | (SEQ ID NO: 343) | (SEQ ID NO: 344) | YLS |
| | | | | (SEQ ID NO: 341) |
| | VL CDR2 | LVS | RLIYLVSKLD | LVSKLDS |
| | | | (SEQ ID NO: 403) | (SEQ ID NO: 385) |
| | VL CDR3 | STHFPL | CQSTHFPL | COSTHFPLT |
| | | (SEQ ID NO: 457) | (SEQ ID NO: 458) | (SEQ ID NO: 456) |

VH Sequence:
QVQLQQSGSELVKPGTSMKLSCKASGYTFTSYNINWVRLRPEQGLEWIGWIFPGDDSIKYNENFRGKATLTTDKSSSTAYMH
LSRLTSDDSAVYFCARSGIFYGNNFAYWGQGTLVTVSA (SEQ ID NO: 23)
VL Sequence:
DVVMTQAPLILSVTIGQPASISCKSSQSLLDGDGETYLSWLLQRPGQSPKRLIYLVSKLDSGVPDRFTGSGSGTDFTLKISR
VEAEDLGVYYCCQSTHFPLTFGAGTKLELK (SEQ ID NO: 24)

TABLE 5
Antibody 1B3 CDR Sequences

| | | Exemplary | IMGT | Kabat |
|---|---|---|---|---|
| VH CDR | VH CDR1 | GFTFTGYNIN | GFTFTGYN | GYNIN |
| Seq. | | (SEQ ID NO: 95) | (SEQ ID NO: 96) | (SEQ ID NO: 97) |
| | VH CDR2 | WIFPGDDNAKYNE | IFPGDDNA | WIFPGDDNAKYNE |
| | | KFKG | (SEQ ID NO: 188) | KFKG |
| | | (SEQ ID NO: 187) | | (SEQ ID NO: 187) |
| | VH CDR3 | TPVLSNYFDY | ARTPVLSNYFDY | TPVLSNYFDY |
| | | (SEQ ID NO: 269) | (SEQ ID NO: 270) | (SEQ ID NO: 269) |
| VL CDR | VL CDR1 | KASQDISKYIS | QDISKY | KASQDISKYIS |
| Seq. | | (SEQ ID NO: 345) | (SEQ ID NO: 346) | (SEQ ID NO: 345) |
| | VL CDR2 | YTSTLQP | YTS | YTSTLQP |
| | | (SEQ ID NO: 404) | | (SEQ ID NO: 404) |
| | VL CDR3 | LQYDNLYT | LQYDNLYT | LOYDNLYT |
| | | (SEQ ID NO: 459) | (SEQ ID NO: 459) | (SEQ ID NO: 459) |
| | | Chothia | Contact | AbM |
| VH CDR | VH CDR1 | GFTFTGY | TGYNIN | GFTFTGYNIN |
| Seq. | | (SEQ ID NO: 98) | (SEQ ID NO: 99) | (SEQ ID NO: 95) |
| | VH CDR2 | PGDD | WIGWIFPGDDNAK | WIFPGDDNAK |
| | | (SEQ ID NO: 184) | (SEQ ID NO: 189) | (SEQ ID NO: 190) |
| | VH CDR3 | PVLSNYFD | ARTPVLSNYFD | TPVLSNYFDY |
| | | (SEQ ID NO: 271) | (SEQ ID NO: 272) | (SEQ ID NO: 269) |
| VL CDR | VL CDR1 | SQDISKY | SKYISWY | KASQDISKYIS |
| Seq. | | (SEQ ID NO: 347) | (SEQ ID NO: 348) | (SEQ ID NO: 345) |
| | VL CDR2 | YTS | LLIHYTSTLQ | YTSTLQP |
| | | | (SEQ ID NO: 406) | (SEQ ID NO: 404) |
| | VL CDR3 | YDNLY | LOYDNLY | LQYDNLYT |
| | | (SEQ ID NO: 460) | (SEQ ID NO: 461) | (SEQ ID NO: 459) |

VH Sequence:
QVHLQQPGAELVKPGASVKLSCKASGFTFTGYNINWVRLRPEQGLEWIGWIFPGDDNAKYNEKFKGKATLTTDKSSNTAYMQ
LSRLTSEDSAVYFCARTPVLSNYFDYWGQGTTLTVSS (SEQ ID NO: 25)
VL Sequence:
DIQMTQSPSSLSASLGGKVTITCKASQDISKYISWYQHKPGKSPRLLIHYTSTLQPGIPSRFSGSGSGRDYSFSISNLEPED
IATYYCLQYDNLYTFGGGTKLEIK
(SEQ ID NO: 26)

TABLE 6
Antibody 6G9 CDR Sequences

| | | Exemplary | IMGT | Kabat |
|---|---|---|---|---|
| VH CDR | VH CDR1 | GYTFTSYWMQ | GYTFTSYW | SYWMQ |
| Seq. | | (SEQ ID NO: 124) | (SEQ ID NO: 73) | (SEQ ID NO: 125) |
| | VH CDR2 | EIDPSDSYTNYNQ | IDPSDSYT | EIDPSDSYTNYNQ |
| | | KFKG | (SEQ ID NO: 211) | KFKG |
| | | (SEQ ID NO: 210) | | (SEQ ID NO: 210) |
| | VH CDR3 | PLDRSAYYFDY | ARPLDRSAYYFDY | PLDRSAYYFDY |
| | | (SEQ ID NO: 289) | (SEQ ID NO: 290) | (SEQ ID NO: 289) |
| VL CDR | VL CDR1 | RASESVDFSGNSF | ESVDFSGNSF | RASESVDFSGNSF |
| Seq. | | MH | (SEQ ID NO: 366) | MH |
| | | (SEQ ID NO: 365) | | (SEQ ID NO: 365) |
| | VL CDR2 | RASNLDS | RAS | RASNLDS |
| | | (SEQ ID NO: 418) | | (SEQ ID NO: 418) |
| | VL CDR3 | QQSNEDPYT | QQSNEDPYT | QQSNEDPYT |
| | | (SEQ ID NO: 474) | (SEQ ID NO: 474) | (SEQ ID NO: 474) |
| | | Chothia | Contact | AbM |
| VH CDR | VH CDR1 | GYTFTSY | TSYWMQ | GYTFTSYWMQ |
| Seq. | | (SEQ ID NO: 75) | (SEQ ID NO: 126) | (SEQ ID NO: 124) |
| | VH CDR2 | PSDS | WIGEIDPSDSYTN | EIDPSDSYTN |
| | | (SEQ ID NO: 212) | (SEQ ID NO: 213) | (SEQ ID NO: 214) |
| | VH CDR3 | LDRSAYYFD | ARPLDRSAYYFD | PLDRSAYYFDY |
| | | (SEQ ID NO: 291) | (SEQ ID NO: 292) | (SEQ ID NO: 289) |
| VL CDR | VL CDR1 | SESVDFSGNSF | DFSGNSFMHWY | RASESVDFSGNSF |
| Seq. | | (SEQ ID NO: 367) | (SEQ ID NO: 368) | MH |
| | | | | (SEQ ID NO: 365) |
| | VL CDR2 | RAS | LLIYRASNLD | RASNLDS |
| | | | (SEQ ID NO: 420) | (SEQ ID NO: 418) |
| | VL CDR3 | SNEDPY | QQSNEDPY | QQSNEDPYT |
| | | (SEQ ID NO: 475) | (SEQ ID NO: 476) | (SEQ ID NO: 474) |

VH Sequence:
QVQLHQPGAELVKPGASVKLSCKTSGYTFTSYWMQWVKQRPGQGLEWIGEIDPSDSYTNYNQKFKGKATLTVDTSSTTAYM
QLSSLTSEDSAVYYCARPLDRSAYYEDYWGQGTTLTVSS (SEQ ID NO: 37)
VL Sequence:
DIVLTQSPASLAVSLGQRATISCRASESVDFSGNSFMHWYQQKPGQPPKLLIYRASNLDSGIPARFSGVGSRTDFTLTINP
VEADDVATYYCQQSNEDPYTFGGGTKLEIE
(SEQ ID NO: 38)

TABLE 7
Antibody 2B11 CDR Sequences

| | | Exemplary | IMGT | Kabat |
|---|---|---|---|---|
| VH CDR | VH CDR1 | GYSITSGYYWN | GYSITSGYY | SGYYWN |
| Seq. | | (SEQ ID NO: 127) | (SEQ ID NO: 128) | (SEQ ID NO: 129) |
| | VH CDR2 | HIANDGSNYYNPF | IANDGSN | HIANDGSNYYNPF |
| | | LKH | (SEQ ID NO: 216) | LKH |
| | | (SEQ ID NO: 215) | | (SEQ ID NO: 215) |
| | VH CDR3 | GGSYFDYVDY | ARGGSYFDYVDY | GGSYFDYVDY |
| | | (SEQ ID NO: 293) | (SEQ ID NO: 294) | (SEQ ID NO: 293) |
| VL CDR | VL CDR1 | RASQDISNYLN | QDISNY | RASQDISNYLN |
| Seq. | | (SEQ ID NO: 369) | (SEQ ID NO: 370) | (SEQ ID NO: 369) |
| | VL CDR2 | YTSRLHS | YTS | YTSRLHS |
| | | (SEQ ID NO: 421) | | (SEQ ID NO: 421) |
| | VL CDR3 | QQGNTLPFT | QQGNTLPFT | QQGNTLPFT |
| | | (SEQ ID NO: 477) | (SEQ ID NO: 477) | (SEQ ID NO: 477) |
| | | Chothia | Contact | AbM |
| VH CDR | VH CDR1 | GYSITSGY | TSGYYWN | GYSITSGYYWN |
| Seq. | | (SEQ ID NO: 130) | (SEQ ID NO: 131) | (SEQ ID NO: 127) |
| | VH CDR2 | NDG | WMGHIANDGSNY | HIANDGSNY |
| | | | (SEQ ID NO: 218) | (SEQ ID NO: 219) |
| | VH CDR3 | GSYFDYVD | ARGGSYFDYVD | GGSYFDYVDY |
| | | (SEQ ID NO: 295) | (SEQ ID NO: 296) | (SEQ ID NO: 293) |
| VL CDR | VL CDR1 | SQDISNY | SNYLNWY | RASQDISNYLN |
| Seq. | | (SEQ ID NO: 371) | (SEQ ID NO: 372) | (SEQ ID NO: 369) |
| | VL CDR2 | YTS | LLIYYTSRLH | YTSRLHS |
| | | | (SEQ ID NO: 422) | (SEQ ID NO: 421) |
| | VL CDR3 | GNTLPF | QQGNTLPF | QQGNTLPFT |
| | | (SEQ ID NO: 478) | (SEQ ID NO: 479) | (SEQ ID NO: 477) |

VH Sequence:
DVQLQESGPGLVKPSQSLSLTCSVTGYSITSGYYWNWIRQFPGNKLEWMGHIANDGSNYYNPFLKHRVSITRDTSKNQFFL
KLNSVTIQDTATYYCARGGSYFDYVDYWGQGTTLTVSS (SEQ ID NO: 39)
VL Sequence:
DIQMTQTTSSLSASLGDRVTINCRASQDISNYLNWYQQKPDGTVKLLIYYTSRLHSGVPSRFSGSGSGTDYSLTITNLEQE
DIATYFCQQGNTLPFTFGSGTKLEIK (SEQ ID NO: 40)

TABLE 8
Antibody 17J16 CDR Sequences

| | | Exemplary | IMGT | Kabat |
|---|---|---|---|---|
| VH CDR | VH CDR1 | GYTFTDYWIH | GYTFTDYW | DYWIH |
| Seq. | | (SEQ ID NO: 69) | (SEQ ID NO: 61) | (SEQ ID NO: 70) |
| | VH CDR2 | YINPNSNYAEYNQ | INPNSNYA | YINPNSNYAEYNQ |
| | | KFKV | (SEQ ID NO: 162) | KFKV |
| | | (SEQ ID NO: 161) | | (SEQ ID NO: 161) |
| | VH CDR3 | FDWNWYFHV | ARFDWNWYFHV | FDWNWYFHV |
| | | (SEQ ID NO: 245) | (SEQ ID NO: 246) | (SEQ ID NO: 245) |
| VL CDR | VL CDR1 | KSSQSLSDSDGKT | QSLSDSDGKTY | KSSQSLSDSDGKT |
| Seq. | | YLN | (SEQ ID NO: 322) | YLN |
| | | (SEQ ID NO: 321) | | (SEQ ID NO: 321) |
| | VL CDR2 | LVSRLGS | LVS | LVSRLGS |
| | | (SEQ ID NO: 391) | | (SEQ ID NO: 391) |
| | VL CDR3 | WQGTHFPQT | WQGTHFPQT | WQGTHFPQT |
| | | (SEQ ID NO: 441) | (SEQ ID NO: 441) | (SEQ ID NO: 441) |
| | | Chothia | Contact | AbM |
| VH CDR | VH CDR1 | GYTFTDY | TDYWIH | GYTFTDYWIH |
| Seq. | | (SEQ ID NO: 49) | (SEQ ID NO: 71) | (SEQ ID NO: 69) |
| | VH CDR2 | PNSN | WIGYINPNSNYAE | YINPNSNYAE |
| | | (SEQ ID NO: 163) | (SEQ ID NO: 164) | (SEQ ID NO: 165) |
| | VH CDR3 | DWNWYFH | ARFDWNWYFH | FDWNWYFHV |
| | | (SEQ ID NO: 247) | (SEQ ID NO: 248) | (SEQ ID NO: 245) |
| VL CDR | VL CDR1 | SQSLSDSDGKTY | SDSDGKTYLNWL | KSSQSLSDSDGKT |
| Seq. | | (SEQ ID NO: 323) | (SEQ ID NO: 324) | YLN |
| | | | | (SEQ ID NO: 321) |
| | VL CDR2 | LVS | RLIYLVSRLG | LVSRLGS |
| | | | (SEQ ID NO: 392) | (SEQ ID NO: 391) |
| | VL CDR3 | GTHFPQ | WQGTHFPQ | WQGTHFPQT |
| | | (SEQ ID NO: 442) | (SEQ ID NO: 443) | (SEQ ID NO: 441) |

VH Sequence:
QVQLQQSGAELAKPGASVKMSCKTSGYTFTDYWIHWVKQRPGQGLEWIGYINPNSNYAEYNQKFKVKATLTADKSSSTAYL
QLSRLTSEDSAVYYCARFDWNWYFHVWGAGSTVTVSS (SEQ ID NO: 13)
VL Sequence:
DVALTQIPLTLSVTVGQPASISCKSSQSLSDSDGKTYLNWLLQKPGQSPKRLIYLVSRLGSGVPDRFTGSGSGADFTLKIS
RVEAEDLGVYYCWQGTHFPQTFGGGTKLEIK (SEQ ID NO: 14)

TABLE 9
Antibody 8D8 CDR Sequences

| | | Exemplary | IMGT | Kabat |
|---|---|---|---|---|
| VH CDR | VH CDR1 | GFSLSRYSVH | GFSLSRYS | RYSVH |
| Seq. | | (SEQ ID NO: 64) | (SEQ ID NO: 65) | (SEQ ID NO: 66) |
| | VH CDR2 | MIWGFGSTDYNSA | IWGFGST | MIWGFGSTDYNSA |
| | | LKS | (SEQ ID NO: 220) | LKS |
| | | (SEQ ID NO: 157) | | (SEQ ID NO: 157) |
| | VH CDR3 | IHTTAGSY | ARIHTTAGSY | IHTTAGSY |
| | | (SEQ ID NO: 241) | (SEQ ID NO: 242) | (SEQ ID NO: 241) |
| VL CDR | VL CDR1 | KASQNVGTNVA | QNVGTN | KASQNVGTNVA |
| Seq. | | (SEQ ID NO: 317) | (SEQ ID NO: 318) | (SEQ ID NO: 317) |
| | VL CDR2 | STSYRYS | STS | STSYRYS |
| | | (SEQ ID NO: 388) | | (SEQ ID NO: 388) |
| | VL CDR3 | HQYNSYPLT | HQYNSYPLT | HQYNSYPLT |
| | | (SEQ ID NO: 438) | (SEQ ID NO: 438) | (SEQ ID NO: 438) |
| | | Chothia | Contact | AbM |
| VH CDR | VH CDR1 | GFSLSRY | SRYSVH | GFSLSRYSVH |
| Seq. | | (SEQ ID NO: 67) | (SEQ ID NO: 68) | (SEQ ID NO: 64) |
| | VH CDR2 | GFG | WLGMIWGFGSTD | MIWGFGSTD |
| | | | (SEQ ID NO: 159) | (SEQ ID NO: 160) |
| | VH CDR3 | HTTAGS | ARIHTTAGS | IHTTAGSY |
| | | (SEQ ID NO: 243) | (SEQ ID NO: 244) | (SEQ ID NO: 241) |
| VL CDR | VL CDR1 | SQNVGTN | GTNVAWY | KASQNVGTNVA |
| Seq. | | (SEQ ID NO: 319) | (SEQ ID NO: 320) | (SEQ ID NO: 317) |
| | VL CDR2 | STS | ALVYSTSYRY | STSYRYS |
| | | | (SEQ ID NO: 390) | (SEQ ID NO: 388) |
| | VL CDR3 | YNSYPL | HQYNSYPL | HQYNSYPLT |
| | | (SEQ ID NO: 439) | (SEQ ID NO: 440) | (SEQ ID NO: 438) |

VH Sequence:
QVQLKESGPGLVAPSQSLSITCTVSGFSLSRYSVHWVRQPPGKGLEWLGMIWGFGSTDYNSALKSRLSITKDNSKSQFFLKM
NSLQTDDTAMYYCARIHTTAGSYWGQGTLVTVSA
(SEQ ID NO: 11)
VL Sequence:
DIVMTQSQKFMSTSIGDRVSVTCKASQNVGTNVAWYQQKPGQSPKALVYSTSYRYSGVPDRFTGSGSGTDFTLTISNVQSED
LAEYFCHQYNSYPLTFGAGTKLELK
(SEQ ID NO: 12)

TABLE 10
Antibody 2B3 CDR Sequences

| | | Exemplary | IMGT | Kabat |
|---|---|---|---|---|
| VH CDR | VH CDR1 | GFTFSDYFMF | GFTFSDYF | DYFMF |
| Seq. | | (SEQ ID NO: 105) | (SEQ ID NO: 106) | (SEQ ID NO: 107) |
| | VH CDR2 | YISNDGDSTYYPD | ISNDGDST | YISNDGDSTYYPD |
| | | TVQG | (SEQ ID NO: 192) | TVQG |
| | | (SEQ ID NO: 191) | | (SEQ ID NO: 191) |
| | VH CDR3 | QGAQATLDY | TRQGAQATLDY | QGAQATLDY |
| | | (SEQ ID NO: 273) | (SEQ ID NO: 274) | (SEQ ID NO: 273) |
| VL CDR | VL CDR1 | SASSSVFYMH | SSVFY | SASSSVFYMH |
| Seq. | | (SEQ ID NO: 349) | (SEQ ID NO: 350) | (SEQ ID NO: 349) |
| | VL CDR2 | STSNLAS | STS | STSNLAS |
| | | (SEQ ID NO: 408) | | (SEQ ID NO: 408) |
| | VL CDR3 | HQWSST | HQWSST | HQWSST |
| | | (SEQ ID NO: 462) | (SEQ ID NO: 462) | (SEQ ID NO: 462) |
| | | Chothia | Contact | AbM |
| VH CDR | VH CDR1 | GFTFSDY | SDYFMF | GFTFSDYFMF |
| Seq. | | (SEQ ID NO: 108) | (SEQ ID NO: 109) | (SEQ ID NO: 105) |
| | VH CDR2 | NDGD | WVAYISNDGDST | YISNDGDSTY |
| | | (SEQ ID NO: 193) | Y | (SEQ ID NO: 195) |
| | | | (SEQ ID NO: 194) | |
| | VH CDR3 | GAQATLD | TRQGAQATLD | QGAQATLDY |
| | | (SEQ ID NO: 275) | (SEQ ID NO: 276) | (SEQ ID NO: 273) |
| VL CDR | VL CDR1 | SSSVFY | FYMHWY | SASSSVFYMH |
| Seq. | | (SEQ ID NO: 351) | (SEQ ID NO: 352) | (SEQ ID NO: 349) |
| | VL CDR2 | STS | LLIYSTSNLA | STSNLAS |
| | | | (SEQ ID NO: 409) | (SEQ ID NO: 408) |
| | VL CDR3 | WSS | HQWSS | HQWSST |
| | | | (SEQ ID NO: 464) | (SEQ ID NO: 462) |

VH Sequence:
EVKLVESGGGLVQPGGSLKLSCAASGFTFSDYFMFWVRQTPEKRLEWVAYISNDGDSTYYPDTVQGR
FTISRDNAKNTLYLQMSRLRSEDTAMYYCTRQGAQATLDYWGQGTTLTVSS (SEQ ID NO: 29)
VL Sequence:
QIVLTQSPAIMSASLGEEITLTCSASSSVFYMHWYQQKSGTSPKLLIYSTSNLASGIPSRFSGSGSG
TFYSLTISSVEAEDAADYYCHQWSSTFGGGTKLEIK
(SEQ ID NO: 30)

TABLE 11
Antibody 24G2 CDR Sequences

| | | Exemplary | IMGT | Kabat |
|---|---|---|---|---|
| VH CDR | VH CDR1 | GYTFTTYWMH | GYTFTTYW | TYWMH |
| Seq. | | (SEQ ID NO: 120) | (SEQ ID NO: 121) | (SEQ ID NO: 122) |
| | VH CDR2 | MIHPNSGSSNYNE | IHPNSGSS | MIHPNSGSSNYNE |
| | | KFKN | (SEQ ID NO: 206) | KFKN |
| | | (SEQ ID NO: 205) | | (SEQ ID NO: 205) |
| | VH CDR3 | SDYGFIPYFDY | ARSDYGFIPYFDY | SDYGFIPYFDY |
| | | (SEQ ID NO: 285) | (SEQ ID NO: 286) | (SEQ ID NO: 285) |
| VL CDR | VL CDR1 | RASQSIGTSIH | QSIGTS | RASQSIGTSIH |
| Seq. | | (SEQ ID NO: 361) | (SEQ ID NO: 362) | (SEQ ID NO: 361) |
| | VL CDR2 | YASESIS | YAS | YASESIS |
| | | (SEQ ID NO: 416) | | (SEQ ID NO: 416) |
| | VL CDR3 | QQSNSWPTFT | QQSNSWPTFT | QQSNSWPTFT |
| | | (SEQ ID NO: 471) | (SEQ ID NO: 471) | (SEQ ID NO: 471) |
| | | Chothia | Contact | AbM |
| VH CDR | VH CDR1 | GYTFTTY | TTYWMH | GYTFTTYWMH |
| Seq. | | (SEQ ID NO: 80) | (SEQ ID NO: 123) | (SEQ ID NO: 120) |
| | VH CDR2 | PNSG | WIGMIHPNSGSSN | MIHPNSGSSN |
| | | (SEQ ID NO: 207) | (SEQ ID NO: 208) | (SEQ ID NO: 209) |
| | VH CDR3 | DYGFIPYFD | ARSDYGFIPYFD | SDYGFIPYFDY |
| | | (SEQ ID NO: 287) | (SEQ ID NO: 288) | (SEQ ID NO: 285) |
| VL CDR | VL CDR1 | SQSIGTS | GTSIHWY | RASQSIGTSIH |
| Seq. | | (SEQ ID NO: 363) | (SEQ ID NO: 364) | (SEQ ID NO: 361) |
| | VL CDR2 | YAS | LLIKYASESI | YASESIS |
| | | | (SEQ ID NO: 417) | (SEQ ID NO: 416) |
| | VL CDR3 | SNSWPTF | QQSNSWPTF | QQSNSWPTFT |
| | | (SEQ ID NO: 472) | (SEQ ID NO: 473) | (SEQ ID NO: 471) |

VH Sequence:
QVQLQQSGAELLKPGASVKLSCKASGYTFTTYWMHWVKQRPGQGLEWIGMIHPNSGSSNYNEKFKNK
ATLTVDKSSSTAYMQLSSLTSEDSAVYFCARSDYGFIPYFDYWGQGTTLTVSS (SEQ ID NO: 35)
VL Sequence:
DILLTQSPAILSVSPGERVSFSCRASQSIGTSIHWYQQRTNGSPRLLIKYASESISGIPSRFSGSGSGT
DFTLIINSVESEDIADYYCQQSNSWPTFTFGAGTKLELK (SEQ ID NO: 36)

TABLE 12
Antibody 5A20 CDR Sequences

| | | Exemplary | IMGT | Kabat |
|---|---|---|---|---|
| VH CDR | VH CDR1 | GYTFTDYWIE | GYTFTDYW | DYWIE |
| Seq. | | (SEQ ID NO: 60) | (SEQ ID NO: 61) | (SEQ ID NO: 62) |
| | VH CDR2 | EILLGSDSIHFNEK | ILLGSDSI | EILLGSDSIHFNEK |
| | | FKG | (SEQ ID NO: 153) | FKG |
| | | (SEQ ID NO: 152) | | (SEQ ID NO: 152) |
| | VH CDR3 | QDWNWYFDV | VRQDWNWYFDV | QDWNWYFDV |
| | | (SEQ ID NO: 237) | (SEQ ID NO: 238) | (SEQ ID NO: 237) |
| VL CDR | VL CDR1 | KSSQSLLDFDGKT | QSLLDFDGKTY | KSSQSLLDFDGKT |
| Seq. | | YLN | (SEQ ID NO: 314) | YLN |
| | | (SEQ ID NO: 313) | | (SEQ ID NO: 313) |
| | VL CDR2 | LVSKLDS | LVS | LVSKLDS |
| | | (SEQ ID NO: 385) | | (SEQ ID NO: 385) |
| | VL CDR3 | WQGTHFPRT | WQGTHFPRT | WQGTHFPRT |
| | | (SEQ ID NO: 435) | (SEQ ID NO: 435) | (SEQ ID NO: 435) |
| | | Chothia | Contact | AbM |
| VH CDR | VH CDR1 | GYTFTDY | TDYWIE | GYTFTDYWIE |
| Seq. | | (SEQ ID NO: 49) | (SEQ ID NO: 63) | (SEQ ID NO: 60) |
| | VH CDR2 | LGSD | WIGEILLGSDSIH | EILLGSDSIH |
| | | (SEQ ID NO: 154) | (SEQ ID NO: 155) | (SEQ ID NO: 156) |
| | VH CDR3 | DWNWYFD | VRQDWNWYFD | QDWNWYFDV |
| | | (SEQ ID NO: 239) | (SEQ ID NO: 240) | (SEQ ID NO: 237) |
| VL CDR | VL CDR1 | SQSLLDFDGKTY | LDFDGKTYLNWY | KSSQSLLDFDGKT |
| Seq. | | (SEQ ID NO: 315) | (SEQ ID NO: 316) | YLN |
| | | | | (SEQ ID NO: 313) |
| | VL CDR2 | LVS | RLFYLVSKLD | LVSKLDS |
| | | | (SEQ ID NO: 387) | (SEQ ID NO: 385) |
| | VL CDR3 | GTHFPR | WQGTHFPR | WQGTHFPRT |
| | | (SEQ ID NO: 436) | (SEQ ID NO: 437) | (SEQ ID NO: 435) |

VH Sequence:
QVQLQQSGPELMKPGASVILSCKAIGYTFTDYWIEWVKERPGHGLEWIGEILLGSDSIHFNEKFKGKAT
ISADTSSNTAYMQLSSLTTEDSAIYYCVRQDWNWYFDVWGTGTTVTVSS (SEQ ID NO: 9)
VL Sequence:
DVVMTQTPLTLSVTIGHPASISCKSSQSLLDFDGKTYLNWLFQRPGQSPKRLFYLVSKLDSGVPDRFTG
SGSGTDFTLKISRVEAEDLGVYYCWQGTHFPRTFGGGTKLEIK (SEQ ID NO: 10)

TABLE 13
Antibody 2B8 CDR Sequences

| | | Exemplary | IMGT | Kabat |
|---|---|---|---|---|
| VH CDR | VH CDR1 | GYTFTTYGMS | GYTFTTYG | TYGMS |
| Seq. | | (SEQ ID NO: 77) | (SEQ ID NO: 78) | (SEQ ID NO: 79) |
| | VH CDR2 | WINTYSGVPTFVD | INTYSGVP | WINTYSGVPTFVD |
| | | DFRG | (SEQ ID NO: 143) | DFRG |
| | | (SEQ ID NO: 171) | | (SEQ ID NO: 171) |
| | VH CDR3 | RSSYYPYWYFDV | ARRSSYYPYWYFD | RSSYYPYWYFDV |
| | | (SEQ ID NO: 253) | V | (SEQ ID NO: 253) |
| | | | (SEQ ID NO: 254) | |
| VL CDR | VL CDR1 | RPSENIYSYLT | ENIYSY | RPSENIYSYLT |
| Seq. | | (SEQ ID NO: 329) | (SEQ ID NO: 330) | (SEQ ID NO: 329) |
| | VL CDR2 | NAQTLAE | NAQ | NAQTLAE |
| | | (SEQ ID NO: 395) | | (SEQ ID NO: 395) |
| | VL CDR3 | QHYYGYPFT | QHYYGYPFT | QHYYGYPFT |
| | | (SEQ ID NO: 447) | (SEQ ID NO: 447) | (SEQ ID NO: 447) |
| | | Chothia | Contact | AbM |
| VH CDR | VH CDR1 | GYTFTTY | TTYGMS | GYTFTTYGMS |
| Seq. | | (SEQ ID NO: 80) | (SEQ ID NO: 81) | (SEQ ID NO: 77) |
| | VH CDR2 | TYSG | WMGWINTYSGVP | WINTYSGVPT |
| | | (SEQ ID NO: 144) | T | (SEQ ID NO: 146) |
| | | | (SEQ ID NO: 145) | |
| | VH CDR3 | SSYYPYWYFD | ARRSSYYPYWYFD | RSSYYPYWYFDV |
| | | (SEQ ID NO: 255) | (SEQ ID NO: 256) | (SEQ ID NO: 253) |
| VL CDR | VL CDR1 | SENIYSY | YSYLTWF | RPSENIYSYLT |
| Seq. | | (SEQ ID NO: 331) | (SEQ ID NO: 332) | (SEQ ID NO: 329) |
| | VL CDR2 | NAQ | LLVYNAQTLA | NAQTLAE |
| | | | (SEQ ID NO: 397) | (SEQ ID NO: 395) |
| | VL CDR3 | YYGYPF | QHYYGYPF | QHYYGYPFT |
| | | (SEQ ID NO: 448) | (SEQ ID NO: 449) | (SEQ ID NO: 447) |

VH Sequence:
QIQLVQSGPELKKPGETVKISCKASGYTFTTYGMSWVKQAPGKIFKWMGWINTYSGVPTFVDDFRGRFAFSLETSAS
TAYLQIGNLKNEDTATYFCARRSSYYPYWYFDVWGTGTTVTVSS
(SEQ ID NO: 17)
VL Sequence:
DIQMTQSPASLSASVGETVTITCRPSENIYSYLTWFQQEQGKSPQLLVYNAQTLAEGVPSRFSGSGSGTHFSLKINS
LQPEDFGTYYCQHYYGYPFTFGSGTKLEIK
(SEQ ID NO: 18)

TABLE 14
Antibody 8C10 CDR Sequences

| | | Exemplary | IMGT | Kabat |
|---|---|---|---|---|
| VH CDR | VH CDR1 | GYTFANYGLT | GYTFANYG | NYGLT |
| Seq. | | (SEQ ID NO: 110) | (SEQ ID NO: 111) | (SEQ ID NO: 112) |
| | VH CDR2 | EIYPGSGHTHYNE | IYPGSGHT | EIYPGSGHTHYNE |
| | | DFKG | (SEQ ID NO: 197) | DFKG |
| | | (SEQ ID NO: 196) | | (SEQ ID NO: 196) |
| | VH CDR3 | RIQLLLPVGGFVY | ARRIQLLLPVGGF | RIQLLLPVGGFVY |
| | | (SEQ ID NO: 277) | VY | (SEQ ID NO: 277) |
| | | | (SEQ ID NO: 278) | |
| VL CDR | VL CDR1 | RASQSISNNLH | QSISNN | RASQSISNNLH |
| Seq. | | (SEQ ID NO: 353) | (SEQ ID NO: 354) | (SEQ ID NO: 353) |
| | VL CDR2 | YASQSIS | YAS | YASQSIS |
| | | (SEQ ID NO: 410) | | (SEQ ID NO: 410) |
| | VL CDR3 | QQSNSWPHT | QQSNSWPHT | QQSNSWPHT |
| | | (SEQ ID NO: 465) | (SEQ ID NO: 465) | (SEQ ID NO: 465) |
| | | Chothia | Contact | AbM |
| VH CDR | VH CDR1 | GYTFANY | ANYGLT | GYTFANYGLT |
| Seq. | | (SEQ ID NO: 113) | (SEQ ID NO: 114) | (SEQ ID NO: 110) |
| | VH CDR2 | PGSG | WIGEIYPGSGHTH | EIYPGSGHTH |
| | | (SEQ ID NO: 198) | (SEQ ID NO: 199) | (SEQ ID NO: 200) |
| | VH CDR3 | IQLLLPVGGFV | ARRIQLLLPVGGF | RIQLLLPVGGFVY |
| | | (SEQ ID NO: 279) | V | (SEQ ID NO: 277) |
| | | | (SEQ ID NO: 280) | |
| VL CDR | VL CDR1 | SQSISNN | SNNLHWY | RASQSISNNLH |
| Seq. | | (SEQ ID NO: 355) | (SEQ ID NO: 356) | (SEQ ID NO: 353) |
| | VL CDR2 | YAS | LLIKYASQSI | YASQSIS |
| | | | (SEQ ID NO: 412) | (SEQ ID NO: 410) |
| | VL CDR3 | SNSWPH | QQSNSWPH | QQSNSWPHT |
| | | (SEQ ID NO: 466) | (SEQ ID NO: 467) | (SEQ ID NO: 465) |

VH Sequence:
QVQLQQSGVELARPGAAVKLSCKASGYTFANYGLTWVKQRTGQGLEWIGEIYPGSGHTHYNEDFKGKA
TLTADRSSSTAYMELRSLTSEDSAVYFCARRIQLLLPVGGFVYWGQGTLVTVSA (SEQ ID NO: 31)
VL Sequence:
DFVLTQSPATLSVTPGDSVSLSCRASQSISNNLHWYQQKSHESPRLLIKYASQSISGIPSRFSGSGSG
TDFTLSINSVETEDFGVYFCQQSNSWPHTFGGGTKLEIK
(SEQ ID NO: 32)

TABLE 15
Antibody 25M22 CDR Sequences

| | | Exemplary | IMGT | Kabat |
|---|---|---|---|---|
| VH CDR | VH CDR1 | GYTFTSYWVN | GYTFTSYW | SYWVN |
| Seq. | | (SEQ ID NO: 72) | (SEQ ID NO: 73) | (SEQ ID NO: 74) |
| | VH CDR2 | RIYPGDGDTNYNG | IYPGDGDT | RIYPGDGDTNYNG |
| | | KFKG | (SEQ ID NO: 167) | KFKG |
| | | (SEQ ID NO: 166) | | (SEQ ID NO: 166) |
| | VH CDR3 | MDY | YAMDY | MDY |
| | | (SEQ ID NO: 249) | (SEQ ID NO: 250) | (SEQ ID NO: 249) |
| | | AYLLRLRRTGYYA | ARAYLLRLRRTGY | AYLLRLRRTGYYA |
| VL CDR | VL CDR1 | KSTKSLLNSDEFT | KSLLNSDEFTY | KSTKSLLNSDEFT |
| Seq. | | YLD | (SEQ ID NO: 326) | YLD |
| | | (SEQ ID NO: 325) | | (SEQ ID NO: 325) |
| | VL CDR2 | LVSNRFS | LVS | LVSNRFS |
| | | (SEQ ID NO: 393) | | (SEQ ID NO: 393) |
| | VL CDR3 | FQSNYLPYT | FQSNYLPYT | FQSNYLPYT |
| | | (SEQ ID NO: 444) | (SEQ ID NO: 444) | (SEQ ID NO: 444) |
| | | Chothia | Contact | AbM |
| VH CDR | VH CDR1 | GYTFTSY | TSYWVN | GYTFTSYWVN |
| Seq. | | (SEQ ID NO: 75) | (SEQ ID NO: 76) | (SEQ ID NO: 72) |
| | VH CDR2 | PGDG | WIGRIYPGDGDTN | RIYPGDGDTN |
| | | (SEQ ID NO: 168) | (SEQ ID NO: 169) | (SEQ ID NO: 170) |
| | VH CDR3 | YLLRLRRTGYYA | ARAYLLRLRRTGYAY | MDY |
| | | MD | LLRLRRTGYYAYAMD | (SEQ ID NO: 249) |
| | | (SEQ ID NO: 251) | (SEQ ID NO: 252) | |
| VL CDR | VL CDR1 | TKSLLNSDEFTY | LNSDEFTYLDWY | KSTKSLLNSDEFT |
| Seq. | | (SEQ ID NO: 327) | (SEQ ID NO: 328) | YLD |
| | | | | (SEQ ID NO: 325) |
| | VL CDR2 | LVS | LLIFLVSNRF | LVSNRFS |
| | | | (SEQ ID NO: 394) | (SEQ ID NO: 393) |
| | VL CDR3 | SNYLPY | FQSNYLPY | FQSNYLPYT |
| | | (SEQ ID NO: 445) | (SEQ ID NO: 446) | (SEQ ID NO: 444) |

VH Sequence:
QVQLQQSGPDLVKPGASVKISCKASGYTFTSYWVNWMKQRPGKGLEWIGRIYPGDGDTNYNGKFKGKATLTAD
KSSSTAYMQLSSLTSEDSAVYFCARAYLLRLRRTGYYAMDYWGQGTSVTVSS (SEQ ID NO: 15)
VL Sequence:
DVVLTQTPLSLPVNIGDQASISCKSTKSLLNSDEFTYLDWYLQKPGQSPQLLIFLVSNRFSGVPDRFSGSGSG
TDFTLKISRVEAEDLGVYYCFQSNYLPYTFGGGTKLEIK
(SEQ ID NO: 16)

TABLE 16
Antibody 12A3 CDR Sequences

| | | Exemplary | IMGT | Kabat |
|---|---|---|---|---|
| VH CDR | VH CDR1 | GYPFTIYGMN | GYPFTIYG | IYGMN |
| Seq. | | (SEQ ID NO: 51) | (SEQ ID NO: 52) | (SEQ ID NO: 53) |
| | VH CDR2 | WINTYSGVPTYAD | INTYSGVP | WINTYSGVPTYAD |
| | | DFKG | (SEQ ID NO: 143) | DFKG |
| | | (SEQ ID NO: 142) | | (SEQ ID NO: 142) |
| | VH CDR3 | ATGNY | ASATGNY | ATGNY |
| | | (SEQ ID NO: 229) | (SEQ ID NO: 230) | (SEQ ID NO: 229) |
| VL CDR | VL CDR1 | RASQDIGSSLN | QDIGSS | RASQDIGSSLN |
| Seq. | | (SEQ ID NO: 305) | (SEQ ID NO: 306) | (SEQ ID NO: 305) |
| | VL CDR2 | ATSSLDS | ATS | ATSSLDS |
| | | (SEQ ID NO: 379) | | (SEQ ID NO: 379) |
| | VL CDR3 | LQYASSPYT | LQYASSPYT | LQYASSPYT |
| | | (SEQ ID NO: 429) | (SEQ ID NO: 429) | (SEQ ID NO: 429) |
| | | Chothia | Contact | AbM |
| VH CDR | VH CDR1 | GYPFTIY | TIYGMN | GYPFTIYGMN |
| Seq. | | (SEQ ID NO: 54) | (SEQ ID NO: 55) | (SEQ ID NO: 51) |
| | VH CDR2 | TYSG | WMGWINTYSGVP | WINTYSGVPT |
| | | (SEQ ID NO: 144) | T | (SEQ ID NO: 146) |
| | | | (SEQ ID NO: 145) | |
| | VH CDR3 | TGN | ASATGN | ATGNY |
| | | | (SEQ ID NO: 232) | (SEQ ID NO: 229) |
| VL CDR | VL CDR1 | SQDIGSS | GSSLNWL | RASQDIGSSLN |
| Seq. | | (SEQ ID NO: 307) | (SEQ ID NO: 308) | (SEQ ID NO: 305) |
| | VL CDR2 | ATS | RLIYATSSLD | ATSSLDS |
| | | | (SEQ ID NO: 381) | (SEQ ID NO: 379) |
| | VL CDR3 | YASSPY | LQYASSPY | LQYASSPYT |
| | | (SEQ ID NO: 430) | (SEQ ID NO: 431) | (SEQ ID NO: 429) |

VH Sequence:
QIQLVQSGPELKKPGETVKISCKASGYPFTIYGMNWVEQAPGKGLKWMGWINTYSGVPTYADDFKGRFAFSL
ETSASTAYLQINNLKDEDTATYFCASATGNYWGQGTTLTVSS (SEQ ID NO: 5)
VL Sequence:
DIQMTQSPSSLSASLGERVSLTCRASQDIGSSLNWLQQEPDGTIKRLIYATSSLDSGVPKRFSGSRSGSDYSL
TISSLESEDFVDYYCLQYASSPYTFGGGTKVEIK
(SEQ ID NO: 6)

TABLE 17
Antibody 19K19 CDR Sequences

| | | Exemplary | IMGT | Kabat |
|---|---|---|---|---|
| VH CDR | VH CDR1 | GYAFTSYWMN | GYAFTSYW | SYWMN |
| Seq. | | (SEQ ID NO: 100) | (SEQ ID NO: 101) | (SEQ ID NO: 102) |
| | VH CDR2 | RIYPGDGDTNYNG | IYPGDGDT | RIYPGDGDTNYNG |
| | | KFKG | (SEQ ID NO: 167) | KFKG |
| | | (SEQ ID NO: 166) | | (SEQ ID NO: 166) |
| | VH CDR3 | MDY | YAMDY | MDY |
| | | (SEQ ID NO: 249) | (SEQ ID NO: 250) | (SEQ ID NO: 249) |
| | | AYLLRLRRTGYYA | ARAYLLRLRRTGY | AYLLRLRRTGYYA |
| VL CDR | VL CDR1 | KSTKSLLNSDEFT | KSLLNSDEFTY | KSTKSLLNSDEFT |
| Seq. | | YLD | (SEQ ID NO: 326) | YLD |
| | | (SEQ ID NO: 325) | | (SEQ ID NO: 325) |
| | VL CDR2 | LVSNRFS | LVS | LVSNRFS |
| | | (SEQ ID NO: 393) | | (SEQ ID NO: 393) |
| | VL CDR3 | FQSNYLPYT | FQSNYLPYT | FQSNYLPYT |
| | | (SEQ ID NO: 444) | (SEQ ID NO: 444) | (SEQ ID NO: 444) |
| | | Chothia | Contact | AbM |
| VH CDR | VH CDR1 | GYAFTSY | TSYWMN | GYAFTSYWMN |
| Seq. | | (SEQ ID NO: 103) | (SEQ ID NO: 104) | (SEQ ID NO: 100) |
| | VH CDR2 | PGDG | WIGRIYPGDGDTN | RIYPGDGDTN |
| | | (SEQ ID NO: 168) | (SEQ ID NO: 169) | (SEQ ID NO: 170) |
| | VH CDR3 | YLLRLRRTGYYA | ARAYLLRLRRTGY | MDY |
| | | MD | AYLLRLRRTGYYAYAMD | (SEQ ID NO: 249) |
| | | (SEQ ID NO: 251) | (SEQ ID NO: 252) | |
| VL CDR | VL CDR1 | TKSLLNSDEFTY | LNSDEFTYLDWY | KSTKSLLNSDEFT |
| Seq. | | (SEQ ID NO: 327) | (SEQ ID NO: 328) | YLD |
| | | | | (SEQ ID NO: 325) |
| | VL CDR2 | LVS | LLIYLVSNRF | LVSNRFS |
| | | | (SEQ ID NO: 407) | (SEQ ID NO: 393) |
| | VL CDR3 | SNYLPY | FQSNYLPY | FQSNYLPYT |
| | | (SEQ ID NO: 445) | (SEQ ID NO: 446) | (SEQ ID NO: 444) |

VH Sequence:
QVQLQQSGPDLVKPGASVKISCKASGYAFTSYWMNWVKQRPGKGLEWIGRIYPGDGDTNYNGKFKGKATLTADKSSSTAYMQ
LSSLTSEDSAVYFCARAYLLRLRRTGYYAMDYWGQGTSVTVSS (SEQ ID NO: 27)
VL Sequence:
DVVLTQTPLSLPVNIGDQASISCKSTKSLLNSDEFTYLDWYLQKPGQSPQLLIYLVSNRFSGVPDRFSGSGSGTDFTLKISR
VEAEDLGVYYCFQSNYLPYTFGGGTKLEIK
(SEQ ID NO: 28)

TABLE 18
Antibody 1C1 CDR Sequences

| | | Exemplary | IMGT | Kabat |
|---|---|---|---|---|
| VH CDR | VH CDR1 | GFSLNDYGVH | GFSLNDYG | DYGVH |
| Seq. | | (SEQ ID NO: 41) | (SEQ ID NO: 42) | (SEQ ID NO: 43) |
| | VH CDR2 | VIWSGGRTDYNA | IWSGGRT | VIWSGGRTDYNA |
| | | AFIS | (SEQ ID NO: 133) | AFIS |
| | | (SEQ ID NO: 132) | | (SEQ ID NO: 132) |
| | VH CDR3 | WALYFLYGGSMD | ARWALYFLYGGS | WALYFLYGGSMD |
| | | Y | MDY | Y |
| | | (SEQ ID NO: 221) | (SEQ ID NO: 222) | (SEQ ID NO: 221) |
| VL CDR | VL CDR1 | RSSQSLVHSSGITY | QSLVHSSGITY | RSSQSLVHSSGITY |
| Seq. | | LH | (SEQ ID NO: 298) | LH |
| | | (SEQ ID NO: 297) | | (SEQ ID NO: 297) |
| | VL CDR2 | KLSNRFS | KLS | KLSNRFS |
| | | (SEQ ID NO: 373) | | (SEQ ID NO: 373) |
| | VL CDR3 | SQSTHVPPWT | SQSTHVPPWT | SQSTHVPPWT |
| | | (SEQ ID NO: 423) | (SEQ ID NO: 423) | (SEQ ID NO: 423) |
| | | Chothia | Contact | AbM |
| VH CDR | VH CDR1 | GFSLNDY | NDYGVH | GFSLNDYGVH |
| Seq. | | (SEQ ID NO: 44) | (SEQ ID NO: 45) | (SEQ ID NO: 41) |
| | VH CDR2 | SGG | WLGVIWSGGRTD | VIWSGGRTD |
| | | | (SEQ ID NO: 135) | (SEQ ID NO: 136) |
| | VH CDR3 | ALYFLYGGSMD | ARWALYFLYGGS | WALYFLYGGSMD |
| | | (SEQ ID NO: 223) | MD | Y |
| | | | (SEQ ID NO: 224) | (SEQ ID NO: 221) |
| VL CDR | VL CDR1 | SQSLVHSSGITY | VHSSGITYLHWY | RSSQSLVHSSGITY |
| Seq. | | (SEQ ID NO: 299) | (SEQ ID NO: 300) | LH |
| | | | | (SEQ ID NO: 297) |
| | VL CDR2 | KLS | LLIYKLSNRF | KLSNRFS |
| | | | (SEQ ID NO: 375) | (SEQ ID NO: 373) |
| | VL CDR3 | STHVPPW | SQSTHVPPW | SQSTHVPPWT |
| | | (SEQ ID NO: 424) | (SEQ ID NO: 425) | (SEQ ID NO: 423) |

VH Sequence:
QMQLKQSGPGLVQPSQSLSITCTVSGFSLNDYGVHWIRQSPGKGLEWLGVIWSGGRTDYNAAFISRLSISKDNSKSQVFFKMS
SLQPQDTAIYYCARWALYFLYGGSMDYWGQGTSVTVSS (SEQ ID NO: 1)
VL Sequence:
DVVLTQTPLSLPVSPGDQASISCRSSQSLVHSSGITYLHWYLQKPGQSPKLLIYKLSNRFSGVPDRFSGSGSGTDFTLKISRV
EAEDLGVYFCSQSTHVPPWTFGGGTKLEIK (SEQ ID NO: 2)

TABLE 19
Antibody 22N5 CDR Sequences

| | | Exemplary | IMGT | Kabat |
|---|---|---|---|---|
| VH CDR | VH CDR1 | GYTFTDYSMH | GYTFTDYS | DYSMH |
| Seq. | | (SEQ ID NO: 82) | (SEQ ID NO: 83) | (SEQ ID NO: 84) |
| | VH CDR2 | WINTETGEPTYAD | INTETGEP | WINTETGEPTYAD |
| | | DFKG | (SEQ ID NO: 173) | DFKG |
| | | (SEQ ID NO: 172) | | (SEQ ID NO: 172) |
| | VH CDR3 | GTLNY | VKGTLNY | GTLNY |
| | | (SEQ ID NO: 257) | (SEQ ID NO: 258) | (SEQ ID NO: 257) |
| VL CDR | VL CDR1 | KASQDIKSYLN | QDIKSY | KASQDIKSYLN |
| Seq. | | (SEQ ID NO: 333) | (SEQ ID NO: 334) | (SEQ ID NO: 333) |
| | VL CDR2 | RTKRLVD | RTK | RTKRLVD |
| | | (SEQ ID NO: 398) | | (SEQ ID NO: 398) |
| | VL CDR3 | LQYVEFPLT | LQYVEFPLT | LQYVEFPLT |
| | | (SEQ ID NO: 450) | (SEQ ID NO: 450) | (SEQ ID NO: 450) |
| | | Chothia | Contact | AbM |
| VH CDR | VH CDR1 | GYTFTDY | TDYSMH | GYTFTDYSMH |
| Seq. | | (SEQ ID NO: 49) | (SEQ ID NO: 85) | (SEQ ID NO: 82) |
| | VH CDR2 | TETG | WMGWINTETGEP | WINTETGEPT |
| | | (SEQ ID NO: 174) | T | (SEQ ID NO: 176) |
| | | | (SEQ ID NO: 175) | |
| | VH CDR3 | TLN | VKGTLN | GTLNY |
| | | | (SEQ ID NO: 260) | (SEQ ID NO: 257) |
| VL CDR | VL CDR1 | SQDIKSY | KSYLNWF | KASQDIKSYLN |
| Seq. | | (SEQ ID NO: 335) | (SEQ ID NO: 336) | (SEQ ID NO: 333) |
| | VL CDR2 | RTK | TLIYRTKRLV | RTKRLVD |
| | | | (SEQ ID NO: 400) | (SEQ ID NO: 398) |
| | VL CDR3 | YVEFPL | LQYVEFPL | LQYVEFPLT |
| | | (SEQ ID NO: 451) | (SEQ ID NO: 452) | (SEQ ID NO: 450) |

VH Sequence:
QNQLVQSGPELKKPGEIVKISCKTSGYTFTDYSMHWVKKTPGKGFKWMGWINTETGEPTYADDFKGRFAFSLETSANT
AHLQITNLKNEDTATYFCVKGTLNYWGQGTTLTVSS (SEQ ID NO: 19)
VL Sequence:
DIKMTQSPSSMYASLGERVTITCKASQDIKSYLNWFQQKPGKSPKTLIYRTKRLVDGVPSRFSGSGSGQDYSLTVSS
LEYDDVGIYYCLQYVEFPLTFGDGTKLELK
(SEQ ID NO: 20)

TABLE 20
Antibody 2A9 CDR Sequences

| | | Exemplary | IMGT | Kabat |
|---|---|---|---|---|
| VH CDR | VH CDR1 | GFTFSTYAMS | GFTFSTYA | TYAMS |
| Seq. | | (SEQ ID NO: 115) | (SEQ ID NO: 116) | (SEQ ID NO: 117) |
| | VH CDR2 | SITSGGTTYYTDSV | ITSGGTT | SITSGGTTYYTDSV |
| | | KG | (SEQ ID NO: 202) | KG |
| | | (SEQ ID NO: 201) | | (SEQ ID NO: 201) |
| | VH CDR3 | DGNFYYYGMDY | ARDGNFYYYGMD | DGNFYYYGMDY |
| | | (SEQ ID NO: 281) | Y | (SEQ ID NO: 281) |
| | | | (SEQ ID NO: 282) | |
| VL CDR | VL CDR1 | KASQNVGTAVA | QNVGTA | KASQNVGTAVA |
| Seq. | | (SEQ ID NO: 357) | (SEQ ID NO: 358) | (SEQ ID NO: 357) |
| | VL CDR2 | SASNRFT | SAS | SASNRFT |
| | | (SEQ ID NO: 413) | | (SEQ ID NO: 413) |
| | VL CDR3 | QQYSSYFT | QQYSSYFT | QQYSSYFT |
| | | (SEQ ID NO: 468) | (SEQ ID NO: 468) | (SEQ ID NO: 468) |
| | | Chothia | Contact | AbM |
| VH CDR | VH CDR1 | GFTFSTY | STYAMS | GFTFSTYAMS |
| Seq. | | (SEQ ID NO: 118) | (SEQ ID NO: 119) | (SEQ ID NO: 115) |
| | VH CDR2 | SGG | WVASITSGGTTY | SITSGGTTY |
| | | | (SEQ ID NO: 203) | (SEQ ID NO: 204) |
| | VH CDR3 | GNFYYYGMD | ARDGNFYYYGMD | DGNFYYYGMDY |
| | | (SEQ ID NO: 283) | (SEQ ID NO: 284) | (SEQ ID NO: 281) |
| VL CDR | VL CDR1 | SQNVGTA | GTAVAWY | KASQNVGTAVA |
| Seq. | | (SEQ ID NO: 359) | (SEQ ID NO: 360) | (SEQ ID NO: 357) |
| | VL CDR2 | SAS | ILIYSASNRF | SASNRFT |
| | | | (SEQ ID NO: 415) | (SEQ ID NO: 413) |
| | VL CDR3 | YSSYF | QQYSSYF | QQYSSYFT |
| | | (SEQ ID NO: 469) | (SEQ ID NO: 470) | (SEQ ID NO: 468) |

VH Sequence:
EVKLVESGGGLVKPGGSLKLSCAASGFTFSTYAMSWVRQTPEKRLEWVASITSGGTTYYTDSVKGRFTISRDNAR
NILYLQMSSLRSEDTAMYYCARDGNFYYYGMDYWGQGTSVTVSS(SEQ ID NO: 33)
VL Sequence:
DIVMTQSQKFMSTSVGDRVSITCKASQNVGTAVAWYQQKPGQSPKILIYSASNRFTGVPDRFTGSGSGTDFTLTI
SNMQSEDLADYFCQQYSSYFTFGGGTKLELK
(SEQ ID NO: 34)

TABLE 21
Antibody 1A3 CDR Sequences

| | | Exemplary | IMGT | Kabat |
|---|---|---|---|---|
| VH CDR | VH | GFTFTDYYMN | GFTFTDYY | DYYMN |
| Seq. | CDR1 | (SEQ ID NO: 488) | (SEQ ID NO: 1795) | (SEQ ID NO: 490) |
| | VH | DIIPNNGVTSYNQ | IIPNNGVT | DIIPNNGVTSYNQ |
| | CDR2 | KFKG (SEQ ID | (SEQ ID NO: 498) | KFKG (SEQ ID |
| | | NO: 497) | | NO: 497) |
| | VH | EWLLRGMDY | AREWLLRGMDY | EWLLRGMDY |
| | CDR3 | (SEQ ID NO: 515) | (SEQ ID NO: 516) | (SEQ ID NO: 515) |
| VL CDR | VL | RSSKSLLHSNGIT | KSLLHSNGITY | RSSKSLLHSNGIT |
| Seq. | CDR1 | YLY (SEQ ID NO: | (SEQ ID NO: 532) | YLY (SEQ ID NO: |
| | | 531) | | 531) |
| | VL | QMSNLAS | QMS | QMSNLAS |
| | CDR2 | (SEQ ID NO: 547) | | (SEQ ID NO: 547) |
| | VL | AQHLELTWT | AQHLELTWT | AQHLELTWT |
| | CDR3 | (SEQ ID NO: 558) | (SEQ ID NO: 558) | (SEQ ID NO: 558) |
| | | Chothia | Contact | AbM |
| VH | VH | GFTFTDY | TDYYMN | GFTFTDYYMN |
| CDR | CDR1 | (SEQ ID NO: 1796) | (SEQ ID NO: 491) | (SEQ ID NO: 488) |
| Seq. | VH | PNNG | WIGDIIPNNGVTS | DIIPNNGVTS |
| | CDR2 | (SEQ ID NO: 499) | (SEQ ID NO: 500) | (SEQ ID NO: 501) |
| | VH | WLLRGMD | AREWLLRGMD | EWLLRGMDY |
| | CDR3 | (SEQ ID NO: 517) | (SEQ ID NO: 518) | (SEQ ID NO: 515) |
| VL CDR | VL | SKSLLHSNGITY | LHSNGITYLYWY | RSSKSLLHSNGIT |
| Seq. | CDR1 | (SEQ ID NO: 533) | (SEQ ID NO: 534) | YLY (SEQ ID NO: |
| | | | | 531) |
| | VL | QMS | LLIYQMSNLA | QMSNLAS |
| | CDR2 | | (SEQ ID NO: 549) | (SEQ ID NO: 547) |
| | VL | HLELTW | AQHLELTW | AQHLELTWT |
| | CDR3 | (SEQ ID NO: 559) | (SEQ ID NO: 560) | (SEQ ID NO: 558) |

VH Sequence:
EVQLQQSGPELVKPGASVKISCKASGFTFTDYYMNWVKQSHGKSLEWIGDIIPNNGVTSYNQKFKGKATLTVDKSS
STAYMELRSLTSEDSAVYYCAREWLLRGMDYWGQGTSVTVSS (SEQ ID NO: 480)
VL Sequence:
DIVMTQAAFSNPVTLGTSASISCRSSKSLLHSNGITYLYWYLQKPGQSPQLLIYQMSNLASGVPDRESSSGSGTDF
TLRISRVEAEDVGVYYCAQHLELTWTFGGGTKLEIK (SEQ ID NO: 481)

TABLE 22
Antibody P1B6 CDR Sequences

| | | Exemplary | IMGT | Kabat |
|---|---|---|---|---|
| VH CDR | VH | GYTFTDYYMN | GYTFTDYY | DYYMN |
| Seq. | CDR1 | (SEQ ID NO: 489) | (SEQ ID NO: 57) | (SEQ ID NO: 490) |
| | VH | DINPNNGGPIYNQ | INPNNGGP | DINPNNGGPIYNQ |
| | CDR2 | KFKG (SEQ ID | (SEQ ID NO: 503) | KFKG (SEQ ID |
| | | NO: 502) | | NO: 502) |
| | VH | SDSAWFTY | ARSDSAWFTY | SDSAWFTY |
| | CDR3 | (SEQ ID NO: 519) | (SEQ ID NO: 520) | (SEQ ID NO: 519) |
| VL CDR | VL | SASSSVSYMY | SSVSY | SASSSVSYMY |
| Seq. | CDR1 | (SEQ ID NO: 535) | (SEQ ID NO: 536) | (SEQ ID NO: 535) |
| | VL | DTSNLAS | DTS | DTSNLAS |
| | CDR2 | (SEQ ID NO: 550) | | (SEQ ID NO: 550) |
| | VL | QQWNSYPPT | QQWNSYPPT | QQWNSYPPT |
| | CDR3 | (SEQ ID NO: 561) | (SEQ ID NO: 561) | (SEQ ID NO: 561) |
| | | Chothia | Contact | AbM |
| VH | VH | GYTFTDY | TDYYMN | GYTFTDYYMN |
| CDR | CDR1 | (SEQ ID NO: 49) | (SEQ ID NO: 491) | (SEQ ID NO: 489) |
| Seq. | VH | PNNG | WIGDINPNNGGPI | DINPNNGGPI |
| | CDR2 | (SEQ ID NO: 499) | (SEQ ID NO: 504) | (SEQ ID NO: 505) |
| | VH | DSAWFT | ARSDSAWFT | SDSAWFTY |
| | CDR3 | (SEQ ID NO: 521) | (SEQ ID NO: 522) | (SEQ ID NO: 519) |
| VL CDR | VL | SSSVSY | SYMYWY | SASSSVSYMY |
| Seq. | CDR1 | (SEQ ID NO: 537) | (SEQ ID NO: 538) | (SEQ ID NO: 535) |
| | VL | DTS | LLIYDTSNLA | DTSNLAS |
| | CDR2 | | (SEQ ID NO: 552) | (SEQ ID NO: 550) |
| | VL | WNSYPP | QQWNSYPP | QQWNSYPPT |
| | CDR3 | (SEQ ID NO: 562) | (SEQ ID NO: 563) | (SEQ ID NO: 561) |

VH Sequence:
EVQLQQSGPELVKPGASVKMSCKASGYTFTDYYMNWVKQTHGKSLEWIGDINPNNGGPIYNQKFKGKATLTVDKSSSTAY
MELRSLTSEDSAVYYCARSDSAWFTYWGQGTLVTVSA (SEQ ID NO: 482)
VL Sequence:
QIVLTQSPAIMSASPGEKVTMTCSASSSVSYMYWYQQKPGSSPRLLIYDTSNLASGVPVRFSGSGSGTFYSITISRMEA
EDAATYYCQQWNSYPPTFGGGTKLEIK
(SEQ ID NO: 483)

TABLE 23
Antibody P8G4 CDR Sequences

| | | Exemplary | IMGT | Kabat |
|---|---|---|---|---|
| VH CDR | VH | GFSLTSYGVH | GFSLTSYG | SYGVH |
| Seq. | CDR1 | (SEQ ID NO: 492) | (SEQ ID NO: 493) | (SEQ ID NO: 494) |
| | VH | VLWSGGSTDYNA | LWSGGST | VLWSGGSTDYNA |
| | CDR2 | AFIS (SEQ ID NO: | (SEQ ID NO: 511) | AFIS (SEQ ID NO: |
| | | 510) | | 510) |
| | VH | NFGDY | ARNFGDY | NFGDY |
| | CDR3 | (SEQ ID NO: 527) | (SEQ ID NO: 528) | (SEQ ID NO: 527) |
| VL CDR | VL | SASSRVSYMH | SRVSY | SASSRVSYMH |
| Seq. | CDR1 | (SEQ ID NO: 543) | (SEQ ID NO: 544) | (SEQ ID NO: 543) |
| | VL | DTSKLAS | DTS | DTSKLAS |
| | CDR2 | (SEQ ID NO: 556) | | (SEQ ID NO: 556) |
| | VL | QQWNNNPPT | QQWNNNPPT | QQWNNNPPT |
| | CDR3 | (SEQ ID NO: 567) | (SEQ ID NO: 567) | (SEQ ID NO: 567) |
| | | Chothia | Contact | AbM |
| VH | VH | GFSLTSY | TSYGVH | GFSLTSYGVH |
| CDR | CDR1 | (SEQ ID NO: 495) | (SEQ ID NO: 496) | (SEQ ID NO: 492) |
| Seq. | VH | GGS | WLGVLWSGGST | VLWSGGSTD |
| | CDR2 | | D (SEQ ID NO: | (SEQ ID NO: 514) |
| | | | 513) | |
| | VH | FGD | ARNFGD | NFGDY |
| | CDR3 | | (SEQ ID NO: 530) | (SEQ ID NO: 527) |
| VL CDR | VL | SSRVSY | SYMHWY | SASSRVSYMH |
| Seq. | CDR1 | (SEQ ID NO: 545) | (SEQ ID NO: 546) | (SEQ ID NO: 543) |
| | VL | DTS | RWIYDTSKLA | DTSKLAS |
| | CDR2 | | (SEQ ID NO: 557) | (SEQ ID NO: 556) |
| | VL | WNNNPP | QQWNNNPP | QQWNNNPPT |
| | CDR3 | (SEQ ID NO: 568) | (SEQ ID NO: 569) | (SEQ ID NO: 567) |

VH Sequence:
QVQLKQSGPGLVQPSQSLSITCTVSGFSLTSYGVHWVRQSPGKGLDWLGVLWSGGSTDYNAAFISRLSISKDNSKSQVFFKM
NSLQADDTAIYYCARNFGDYWGQGTSVTVSS (SEQ ID NO: 486)
VL Sequence:
QIVLTQSPAIMSASPGEKVTMTCSASSRVSYMHWYQQKSGTSPKRWIYDTSKLASGVPARFSGSGSGTSYSLTISSMEAED
AATYYCQQWNNNPPTFGAGTTLELK
(SEQ ID NO: 487)

TABLE 24
Antibody P1H8 CDR Sequences

| | | Exemplary | IMGT | Kabat |
|---|---|---|---|---|
| VH CDR | VH | GYTFTDYYMN | GYTFTDYY | DYYMN |
| Seq. | CDR1 | (SEQ ID NO: 489) | (SEQ ID NO: 57) | (SEQ ID NO: 490) |
| | VH | DINPNNGGTTYN | INPNNGGT | DINPNNGGTTYN |
| | CDR2 | QKFKG (SEQ ID | (SEQ ID NO: 507) | QKFKG (SEQ ID |
| | | NO: 506) | | NO: 506) |
| | VH | QGPWYFDV | ARQGPWYFDV | QGPWYFDV |
| | CDR3 | (SEQ ID NO: 523) | (SEQ ID NO: 524) | (SEQ ID NO: 523) |
| VL CDR | VL | RSSQTIVHSNGYT | QTIVHSNGYTY | RSSQTIVHSNGYT |
| Seq. | CDR1 | YLE (SEQ ID NO: | (SEQ ID NO: 540) | YLE (SEQ ID NO: |
| | | 539) | | 539) |
| | VL | KVSNRFS | KVS | KVSNRFS |
| | CDR2 | (SEQ ID NO: 553) | | (SEQ ID NO: 553) |
| | VL | FQGSHVPWT | FQGSHVPWT | FQGSHVPWT |
| | CDR3 | (SEQ ID NO: 564) | (SEQ ID NO: 564) | (SEQ ID NO: 564) |
| | | Chothia | Contact | AbM |
| VH | VH | GYTFTDY | TDYYMN | GYTFTDYYMN |
| CDR | CDR1 | (SEQ ID NO: 49) | (SEQ ID NO: 491) | (SEQ ID NO: 489) |
| Seq. | VH | PNNG | WIGDINPNNGGT | DINPNNGGTT |
| | CDR2 | (SEQ ID NO: 499) | T (SEQ ID NO: | (SEQ ID NO: 509) |
| | | | 508) | |
| | VH | GPWYFD | ARQGPWYFD | QGPWYFDV |
| | CDR3 | (SEQ ID NO: 525) | (SEQ ID NO: 526) | (SEQ ID NO: 523) |
| VL CDR | VL | SQTIVHSNGYTY | VHSNGYTYLEWY | RSSQTIVHSNGYT |
| Seq. | CDR1 | (SEQ ID NO: 541) | (SEQ ID NO: 542) | YLE (SEQ ID NO: |
| | | | | 539) |
| | VL | KVS | LLIYKVSNRF | KVSNRFS |
| | CDR2 | | (SEQ ID NO: 555) | (SEQ ID NO: 553) |
| | VL | QGSHVPW | FQGSHVPW | FQGSHVPWT |
| | CDR3 | (SEQ ID NO: 565) | (SEQ ID NO: 566) | (SEQ ID NO: 564) |

VH Sequence:
EVQLQQSGPELVKPGASVKISCKASGYTFTDYYMNWVKQSHGKSLEWIGDINPNNGGTTYNQKFKGKATLTVDKSSSTAYMEL
RSLTSEDSAVYYCARQGPWYFDVWGTGTTVTVSS(SEQ ID NO: 484)
VL Sequence:
DVLMTQTPLSLPVSLGDQASISCRSSQTIVHSNGYTYLEWYLQKPGQSPKLLIYKVSNRFSGVPDRFSGSGSGTDFTLKISRV
EAEDLGVYYCFQGSHVPWTFGGGTKLE
IK (SEQ ID NO: 485)

CDRs of an antibody are defined using a variety of methods/systems by those skilled in the art. These systems and/or definitions have been developed and refined over a number of years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia. Software programs (e.g., abYsis) are available and known to those of skill in the art for analysis of antibody sequences and determination of CDRs. It will be understood that reference in this disclosure to a VH CDR or VH CDRs and/or a VL CDR or VL CDRs of a specific antibody, including a specific VH amino acid sequence and/or a VL amino acid sequence, will encompass all CDR definitions as known to those of skill in the art.

In some instances, a humanized version of an antibody described herein comprises a VH comprising a VH CDR1, a VH CDR2, a VH CDR3, and a VL comprising a VL CDR1, a VL CDR2, and a VL CDR3, wherein the VH CDR1, VH CDR2, and VH CDR3 are from the amino acid sequence of the VH of the antibody described herein, and the VL CDR1, VL CDR2 and VL CDR3 are from the amino acid sequence of the VL of the antibody described herein (see Tables 1-24). For example, in some instances, the antibody comprises (i) a VH comprising the VH CDR1, the VH CDR2, the VH CDR3 from the amino acid sequence set forth in SEQ ID NO:3; and (ii) a VL comprising the VL CDR1, the VL CDR2, and the VL CDR3 from the amino acid sequence set forth in SEQ ID NO:4 (i.e., the six CDRs of antibody 3P10 of Table 1). As another example, in some instances, the antibody comprises (i) a VH comprising the VH CDR1, the VH CDR2, the VH CDR3 from the amino acid sequence set forth in SEQ ID NO:1982; and (ii) a VL comprising the VL CDR1, the VL CDR2, and the VL CDR3 from the amino acid sequence set forth in SEQ ID NO:1997 (i.e., the six CDRs of a humanized 3P10 antibody of Table 1A). In another example, in some instances, the antibody comprises (i) a VH comprising the VH CDR1, the VH CDR2, the VH CDR3 from the amino acid sequence set forth in any one of SEQ ID NOs:1978-1988; and (ii) a VL comprising the VL CDR1, the VL CDR2, and the VL CDR3 from the amino acid sequence set forth in any one of SEQ ID NOs:1990-2000 (i.e., the six CDRs of a humanized 3P10 antibody of Table 1A). In another example, in some instances, the antibody comprises (i) a VH comprising the VH CDR1, the VH CDR2, the VH CDR3 from the amino acid sequence set forth in SEQ ID NO: 7; and (ii) a VL comprising the VL CDR1, the VL CDR2, and the VL CDR3 from the amino acid sequence set forth in SEQ ID NO: 8 (i.e., the six CDRs of antibody 5F12). In another example, in some instances, the antibody comprises (i) a VH comprising the VH CDR1, the VH CDR2, the VH CDR3 from the amino acid sequence set forth in any one of SEQ ID NOs: 1958-1965; and (ii) a VL comprising the VL CDR1, the VL CDR2, and the VL CDR3 from the amino acid sequence set forth in any one of SEQ ID NOs: 1967-1976 (i.e., the six CDRs of a humanized 5F12 antibody).

In some instances, the antibody comprises (i) a VH comprising a VH CDR1, a VH CDR2, and a VH CDR3 defined by any one of the CDR definitions from any one of Tables 1-24, respectively; and (ii) a VL comprising a VL CDR1, a VL CDR2, and a VL CDR3 defined by any one of the CDR definitions from any one of Tables 1-24, respectively. For example, in some instances, the antibody comprises (i) a VH comprising a VH CDR1, a VH CDR2, and a VH CDR3 defined by any one of the CDR definitions from Table 1; and (ii) a VL comprising a VL CDR1, a VL CDR2, and a VL CDR3 defined by any one of the CDR definitions from Table 1. In another example, in some instances, the antibody comprises (i) a VH comprising a VH CDR1, a VH CDR2, and a VH CDR3 defined by any one of the CDR definitions from Table 2; and (ii) a VL comprising a VL CDR1, a VL CDR2, and a VL CDR3 defined by any one of the CDR definitions from Table 2.

In some instances, the CDRs of the antibody are according to the Exemplary designation. See Tables 1-24.

In some instances, the CDRs of the antibody are according to the IMGT designation. See Tables 1-24.

In some instances, the CDRs of the antibody are according to the Kabat designation. See Tables 1-24.

In some instances, the CDRs of the antibody are according to the Chothia designation. See Tables 1-24.

In some instances, the CDRs of the antibody are according to the Contact designation. See Tables 1-24.

In some instances, the CDRs of the antibody are according to the AbM designation. See Tables 1-24.

In some instances, the antibody comprises: (i) a VH comprising a VH CDR1 comprising the amino acid sequence set forth in SEQ ID NO:46, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426.

In some instances, the antibody comprises: (i) a VH comprising a VH CDR1 comprising the amino acid sequence set forth in SEQ ID NO:47, a VH CDR2 comprising the amino acid sequence set forth in SEQ ID NO:138, and a VH CDR3 comprising the amino acid sequence set forth in SEQ ID NO:226; and (ii) a VL comprising a VL CDR1 comprising the amino acid sequence set forth in SEQ ID NO:302, a VL CDR2 comprising the amino acid sequence set forth in SEQ ID NO:377, and a VL CDR3 comprising the amino acid sequence set forth in SEQ ID NO:426.

In some instances, the antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426.

In some instances, the antibody comprises: (i) a VH comprising a VH CDR1 comprising the amino acid sequence set forth in SEQ ID NO:49, a VH CDR2 comprising the amino acid sequence set forth in SEQ ID NO:139, and a VH CDR3 comprising the amino acid sequence set forth in SEQ ID NO:227; and (ii) a VL comprising a VL CDR1 comprising the amino acid sequence set forth in SEQ ID NO:303, a VL CDR2 comprising the amino acid sequence set forth in SEQ ID NO:377, and a VL CDR3 comprising the amino acid sequence set forth in SEQ ID NO:427.

In some instances, the antibody comprises: (i) a VH comprising a VH CDR1 comprising the amino acid sequence set forth in SEQ ID NO:50, a VH CDR2 comprising the amino acid sequence set forth in SEQ ID NO:140, and a VH CDR3 comprising the amino acid sequence set forth in SEQ ID NO:228; and (ii) a VL comprising a VL CDR1 comprising the amino acid sequence set forth in SEQ ID NO:304, a VL CDR2 comprising the amino acid sequence set forth in SEQ ID NO:378, and a VL CDR3 comprising the amino acid sequence set forth in SEQ ID NO:428.

In some instances, the antibody comprises: (i) a VH comprising a VH CDR1 comprising the amino acid sequence set forth in SEQ ID NO:46, a VH CDR2 comprising the amino acid sequence set forth in SEQ ID NO: 141, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426.

In some instances, the antibody comprises a humanized framework region (FR) sequence, e.g., as described herein. In certain embodiments, the antibody described herein comprises a VH comprising a VH FR1, a VH FR2, a VH FR3 and a VH FR4 amino acid sequence described in International Patent Application Publication No. WO 2017/172260, which is incorporated by reference herein in its entirety; and/or (b) a VL comprising a VL FR1, a VL FR2, a VL FR3 and a VL FR4 amino acid sequence described in International Patent Application Publication No. WO 2017/172260.

In some instances, the antibody comprises a VH comprising the VH CDR1, the VH CDR2, and the VH CDR3 of antibody 3P10 (or a humanized version thereof), and wherein the VH comprises an amino acid sequence having at least at least 75%, at least 80%, at least 85%, at least 90%, at least 95%, at least 96%, at least 97%, at least 98%, or at least 99% sequence identity to the amino acid sequence set forth in SEQ ID NO:3. In some instances, the antibody comprises a VH comprising the VH CDR1, the VH CDR2, and the VH CDR3 of antibody 3P10 (or a humanized version thereof), and wherein the VH comprises an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:1982.

In some instances, the antibody comprises a VL comprising the VL CDR1, the VL CDR2, and the VL CDR3 of antibody 3P10 (or a humanized version thereof), and wherein the VL comprises an amino acid sequence having at least at least 75%, at least 80%, at least 85%, at least 90%, at least 95%, at least 96%, at least 97%, at least 98%, or at least 99% sequence identity to the amino acid sequence set forth in SEQ ID NO:4. In some instances, the antibody comprises a VL comprising the VL CDR1, the VL CDR2, and the VL CDR3 of antibody 3P10 (or a humanized version thereof), and wherein the VL comprises an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:1997.

In some instances, the antibody comprises: (i) a VH comprising the VH CDR1, the VH CDR2, and the VH CDR3 of antibody 3P10 (or a humanized version thereof), and wherein the VH comprises an amino acid sequence having at least at least 75%, at least 80%, at least 85%, at least 90%, at least 95%, at least 96%, at least 97%, at least 98%, or at least 99% sequence identity to the amino acid sequence set forth in SEQ ID NO:3; and a VL comprising the VL CDR1, the VL CDR2, and the VL CDR3 of antibody 3P10 (or a humanized version thereof), and wherein the VL comprises an amino acid sequence having at least at least 75%, at least 80%, at least 85%, at least 90%, at least 95%, at least 96%, at least 97%, at least 98%, or at least 99% sequence identity to the amino acid sequence set forth in SEQ ID NO:4. In some instances, the antibody comprises: (i) a VH comprising the VH CDR1, the VH CDR2, and the VH CDR3 of antibody 3P10 (or a humanized version thereof), and wherein the VH comprises an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:1982; and (ii) a VL comprising the VL CDR1, the VL CDR2, and the VL CDR3 of antibody 3P10 (or a humanized version thereof), and wherein the VL comprises an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:1997.

In some instances, the antibody comprises a VH comprising the amino acid sequence set forth in any one of SEQ ID NOs:1978-1988. In some instances, the antibody comprises a VH comprising the amino acid sequence set forth in SEQ ID NO:1982.

In some instances, the antibody comprises a VL comprising the amino acid sequence set forth in any one of SEQ ID NOs:1990-2000. In some instances, the antibody comprises a VL comprising the amino acid sequence set forth in SEQ ID NO:1997.

In some instances, the antibody comprises: (i) a VH comprising the amino acid sequence set forth in any one of SEQ ID NOs:1978-1988; and (ii) a VL comprising the amino acid sequence set forth in any one of SEQ ID NOs:1990-2000. In some instances, the antibody comprises: (i) a VH comprising the amino acid sequence set forth in SEQ ID NO: 1982; and (ii) a VL comprising the amino acid sequence set forth in SEQ ID NO:1997.

In some instances, the antibody comprises a VH comprising the VH CDR1, the VH CDR2, and the VH CDR3 of an antibody described in any one of Tables 1-24, and wherein the VH comprises an amino acid sequence having at least at least 75%, at least 80%, at least 85%, at least 90%, at least 95%, at least 96%, at least 97%, at least 98%, or at least 99% sequence identity to a VH set forth in any one of Tables 1-24, respectively. In some instances, the antibody comprises a VH comprising the VH CDR1, the VH CDR2, and the VH CDR3 of an antibody described in Table 2, and wherein the VH comprises an amino acid sequence having at least at least 75%, at least 80%, at least 85%, at least 90%, at least 95%, at least 96%, at least 97%, at least 98%, or at least 99% sequence identity to a VH set forth in Table 2.

In some instances, the antibody comprises a VL comprising the VL CDR1, the VL CDR2, and the VL CDR3 of an antibody described in any one of Tables 1-24, and wherein the VL comprises an amino acid sequence having at least at least 75%, at least 80%, at least 85%, at least 90%, at least 95%, at least 96%, at least 97%, at least 98%, or at least 99% sequence identity to a VL set forth in any one of Tables 1-24, respectively. In some instances, the antibody comprises a VL comprising the VL CDR1, the VL CDR2, and the VL CDR3 of an antibody described in Table 2, and wherein the VL comprises an amino acid sequence having at least at least 75%, at least 80%, at least 85%, at least 90%, at least 95%, at least 96%, at least 97%, at least 98%, or at least 99% sequence identity to a VL set forth in Table 2.

In some instances, the antibody comprises: (i) a VH comprising the VH CDR1, the VH CDR2, and the VH CDR3 of an antibody described in any one of Tables 1-24, and wherein the VH comprises an amino acid sequence having at least at least 75%, at least 80%, at least 85%, at least 90%, at least 95%, at least 96%, at least 97%, at least 98%, or at least 99% sequence identity to a VH set forth in any one of Tables 1-24, respectively; and a VL comprising the VL CDR1, the VL CDR2, and the VL CDR3 of an antibody described in any one of Tables 1-24, respectively, and wherein the VL comprises an amino acid sequence having at least at least 75%, at least 80%, at least 85%, at least 90%, at least 95%, at least 96%, at least 97%, at least 98%, or at least 99% sequence identity to a VL set forth in any one of Tables 1-24, respectively. In some instances, the antibody comprises: (i) a VH comprising the VH CDR1, the VH CDR2, and the VH CDR3 of the antibody described in Table 2, and wherein the VH comprises an amino acid sequence having at least at least 75%, at least 80%, at least 85%, at least 90%, at least 95%, at least 96%, at least 97%, at least 98%, or at least 99% sequence identity to the VH set forth Table 2; and a VL comprising the VL CDR1, the VL CDR2, and the VL CDR3 of the antibody described in Table 2, and wherein the VL comprises an amino acid sequence having at least at least 75%, at least 80%, at least 85%, at least 90%, at least 95%, at least 96%, at least 97%, at least 98%, or at least 99% sequence identity to a VL set forth Table 2.

In some instances, the antibody comprises a VH comprising a humanized version of a VH described in any one of Tables 1-24.

In some instances, the antibody comprises a VH comprising a humanized version of the amino acid sequence set forth in SEQ ID NO:7. In some instances, the antibody comprises a VH comprising the amino acid sequence set forth in any one of SEQ ID NOs:1958-1965.

In some instances, the antibody comprises a VL comprising a humanized version of a VL described in any one of Tables 1-24.

In some instances, the antibody comprises a VL comprising a humanized version of the amino acid sequence set forth in SEQ ID NO:8. In some instances, the antibody comprises a VL comprising the amino acid sequence set forth in any one of SEQ ID NOs:1967-1976.

In some instances, the antibody comprises: (i) a VH comprising a humanized version of a VH described in any one of Tables 1-24, and (ii) a VL comprising a humanized version of a VL described in any one of Tables 1-24, respectively.

In some instances, the antibody comprises: (i) a VH comprising a humanized version of the amino acid sequence set forth in SEQ ID NO:7; and (ii) a VL comprising a humanized version of the amino acid sequence set forth in SEQ ID NO:8. In some instances, the antibody comprises: (i) a VH comprising the amino acid sequence set forth in any one of SEQ ID NOs:1958-1965; and (ii) a VL comprising the amino acid sequence set forth in any one of SEQ ID NOs:1967-1976.

In some instances, the antibody comprises a heavy chain comprising a VH comprising the VH CDR1, the VH CDR2, and the VH CDR3 from the amino acid sequence set forth in SEQ ID NO:1982, and wherein the heavy chain comprises an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:2010.

In some instances, the antibody comprises a light chain comprising a VL comprising the VL CDR1, the VL CDR2, and the VL CDR3 from the amino acid sequence set forth in SEQ ID NO:1997, and wherein the light chain comprises an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:2012.

In some instances, the antibody comprises: (i) a heavy chain comprising a VH comprising the VH CDR1, the VH CDR2, and the VH CDR3 from the amino acid sequence set forth in SEQ ID NO:1982, and wherein the heavy chain comprises an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:2010; and (ii) a light chain comprising a VL comprising the VL CDR1, the VL CDR2, and the VL CDR3 from the amino acid sequence set forth in SEQ ID NO:1997, and wherein the light chain comprises an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:2012.

In some instances, the antibody comprises a heavy chain comprising the amino acid sequence set forth in SEQ ID NO:2010. In some instances, the antibody comprises a heavy chain consisting of the amino acid sequence set forth in SEQ ID NO:2010.

In some instances, the antibody comprises a light chain comprising the amino acid sequence set forth in SEQ ID NO:2012. In some instances, the antibody comprises a light chain consisting of the amino acid sequence set forth in SEQ ID NO:2012.

In some instances, the antibody comprises: (i) a heavy chain comprising the amino acid sequence set forth in SEQ ID NO:2010; and (ii) a light chain comprising the amino acid sequence set forth in SEQ ID NO:2012. In some instances, the antibody comprises: (i) a heavy chain consisting of the amino acid sequence set forth in SEQ ID NO:2010; and (ii) a light chain consisting of the amino acid sequence set forth in SEQ ID NO:2012.

In certain instances, the antibody is an antibody described in International Patent Application Publication No. WO 2017/172260, which is incorporated by reference herein in its entirety. In certain instances, the antibody is a humanized version of an antibody described in International Patent Application Publication No. WO 2017/172260, which is incorporated by reference herein in its entirety.

In some instances, the antibody specifically binds to the same epitope as an antibody described in International Patent Application Publication No. WO 2017/172260, which is incorporated by reference herein in its entirety.

In some instances, the antibody competes with an antibody described in International Patent Application Publication No. WO 2017/172260, which is incorporated by reference herein in its entirety, for binding to human GFRAL.

In some instances, the antibody specifically binds to the same epitope as an antibody having the VH and VL of any one of Tables 1-24. In some instances, the antibody specifically binds to the same epitope as antibody 3P10 (see Table 1). In some instances, the antibody specifically binds to the same epitope as antibody Hz3P10 (see Table IA). In some instances, the antibody specifically binds to the same epitope as antibody 5F12 (see Table 2).

In some instances, the antibody competes with an antibody described in any one of Tables 1-24 for binding to human GFRAL. In some instances, the antibody competes with an antibody having the VH and VL of an antibody described in any one of Tables 1-24 for binding to human GFRAL. In some instances, the antibody competes with antibody 3P10 (see Table 1) for binding to human GFRAL. In some instances, the antibody competes with antibody Hz3P10 (see Table 1A) for binding to human GFRAL. In some instances, the antibody competes with antibody 5F12 (see Table 2) for binding to human GFRAL.

In some instances, the antibody specifically binds to human GFRAL within amino acid residues 220-316 of the amino acid sequence set forth in SEQ ID NO: 1797. Nonlimiting examples of antibodies that bind to human GFRAL within amino acid residues 220-316 of the amino acid sequence set forth in SEQ ID NO: 1797 include 5F12, 3P10, 6G9, 6N16, 2B11, and 1B3 (see the working examples of International Patent Application Publication No. WO 2017/172260, which is incorporated by reference herein in its entirety).

In some instances, the antibody specifically binds to human GFRAL at one or more (e.g., 1, 2, 3, 4, 5, 6, 7, 8, 9, 10, 11, 12, 13, 14, 15, 16, 17, 18, 19) residues selected from the group consisting of: Met214, Pro216, Pro217, Gln290, Cys291, Thr292, Cys293, Arg294, Thr295, Ile296, Thr297, Gln298, Ser299, Glu301, Lys305, Gln308, His309, His312, and Ser315 of the amino acid sequence set forth in SEQ ID NO: 1797. In some instances, the antibody specifically binds to human GFRAL at Thr297, Gln298, and Ser299 of the amino acid sequence set forth in SEQ ID NO:1797.

In some instances, the antibody specifically binds to human GFRAL within amino acid residues 20-130 of the amino acid sequence set forth in SEQ ID NO: 1797. In some instances, the antibody does not inhibit binding of human GDF15 to human GFRAL. Nonlimiting examples of antibodies that bind to human GFRAL within amino acid residues 20-130 of the amino acid sequence set forth in SEQ ID NO: 1797 include 1A3, P1B6, P1H8, P8G4 (see the working examples of International Patent Application Publication No. WO 2017/172260, which is incorporated by reference herein in its entirety).

In some instances, the antibody specifically binds to human GFRAL within amino acid residues 131-210 of the amino acid sequence set forth in SEQ ID NO: 1797. In some instances, the antibody inhibits binding of human GDF15 to human GFRAL. Nonlimiting examples of antibodies that bind to human GFRAL within amino acid residues 131-210 of the amino acid sequence set forth in SEQ ID NO: 1797 include 17J16, 8C10, 25M22, 12A3, 19K19, 1C1, 8D8, 22N5, 2A9, and 5A20 (see the working examples of International Patent Application Publication No. WO 2017/172260, which is incorporated by reference herein in its entirety).

In some instances, the antibody specifically binds to one or more residues selected from the group consisting of GLY140, LEU148, ALA149, ALA146, VAL142, ASN145, VAL139, ALA135, GLU136, LEU152, LEU132, SER201, ALA204, LEU205, LYS153, ILE196, PRO197, and GLN200 of a GFRAL protein (SEQ ID NO: 1797). In some instances, the antibody inhibits binding of human GFRAL to human GDF15.

In some instances, the antibody specifically binds to one or more residues selected from the group consisting of SER156, GLN147, SER150, TYR151, ALA154, CYS155, PHE174, TYR175, ALA137, CYS138, ASP141, VAL143, CYS144, LEU186, CYS189, CYS191, ALA192, GLN193, SER194, ASP195, CYS198, GLN199, LYS202, GLU203, HIS206, SER207, SER130, CYS131, LEU132, GLU133, and VAL134 of a GFRAL protein (SEQ ID NO: 1797). In some instances, the antibody inhibits binding of human GFRAL to human GDF15.

In some instances, the antibody specifically binds to one or more residues selected from the group consisting of SER156, GLN147, LEU148, ALA149, SER150, TYR151, LEU152, LYS153, ALA154, CYS155, PHE174, TYR175, GLU136, ALA137, CYS138, VAL139, GLY140, ASP141, VAL142, VAL143, CYS144, ASN145, ALA146, LEU186, CYS189, CYS191, ALA192, GLN193, SER194, ASP195, ILE196, PRO197, CYS198, GLN199, GLN200, SER201, LYS202, GLU203, ALA204, LEU205, HIS206, SER207, SER130, CYS131, LEU132, GLU133, VAL134, and ALA135 of a GFRAL protein (SEQ ID NO: 1797). In some instances, the antibody inhibits binding of human GFRAL to human GDF15.

In some instances, the antibody specifically binds to one or more residues selected from the group consisting of GLU136, ALA137, VAL139, GLY140, ASP141, VAL142, VAL143, CYS144, ASN145, ALA146, GLN147, PHE173, ASN177, ILE178, PRO179, ASN181, ILE182, and MET185 of a GFRAL protein (SEQ ID NO: 1797). In some instances, the antibody inhibits binding of human GFRAL to GDF15 human.

In some instances, the antibody specifically binds to one or more residues selected from the group consisting of LEU132, GLU133, VAL134, ALA135, CYS138, LEU148, ALA149, SER150, TYR151, PHE174, TYR175, ALA169, ALA170, ILE171, ARG172, GLN176, PHE180, ALA183, GLN184, LEU186, ALA187, PHE188, and CYS189 of a GFRAL protein (SEQ ID NO: 1797). In some instances, the antibody inhibits binding of human GFRAL to human GDF15.

In some instances, the antibody specifically binds to one or more residues selected from the group consisting of LEU132, GLU133, VAL134, ALA135, GLU136, ALA137, CYS138, VAL139, GLY140, ASP141, VAL142, VAL143, CYS144, ASN145, ALA146, GLN147, LEU148, ALA149, SER150, TYR151, PHE174, TYR175, ALA169, ALA170, ILE171, ARG172, PHE173, GLN176, ASN177, ILE178, PRO179, PHE180, ASN181, ILE182, ALA183, GLN184, MET185, LEU186, ALA187, PHE188, and CYS189 of a GFRAL protein (SEQ ID NO: 1797). In some instances, the antibody inhibits binding of human GFRAL to human GDF15.

In some instances, the antibody specifically binds to one or more residues selected from the group consisting of MET214, PRO216, PRO217, GLN290, CYS291, THR292, CYS293, ARG294, THR295, ILE296, THR297, GLN298, SER299, GLU301, LYS305, GLN308, HIS309, HIS312, and SER315 of the GFRAL protein (SEQ ID NO: 1797). In some instances, the antibody inhibits binding of human GFRAL to human RET.

In some instances, the antibody is a humanized antibody.

Various methods for generating humanized antibodies are known in the art. In some embodiments, a humanized antibody comprises one or more amino acid residues that have been introduced into it from a source that is non-human. In some embodiments, humanization is performed by substituting one or more non-human CDR sequences for the corresponding CDR sequences of a human antibody. In some embodiments, the humanized antibodies are constructed by substituting all six CDRs of a non-human antibody (e.g., a mouse antibody) for the corresponding CDRs of a human antibody.

The choice of which human heavy chain variable region and/or light chain variable region is used for generating humanized antibodies can be made based on a variety of factors and by a variety of methods known in the art. In some embodiments, the “best-fit” method is used where the sequence of the variable region of a non-human (e.g., rodent) antibody is screened against the entire library of known human variable region sequences. The human sequence that is most similar to that of the non-human (e.g., rodent) sequence is selected as the human variable region framework for the humanized antibody. In some embodiments, a particular variable region framework derived from a consensus sequence of all human antibodies of a particular subgroup of light or heavy chains is selected as the variable region framework. In some embodiments, the variable region framework sequence is derived from the consensus sequences of the most abundant human subclasses. In some embodiments, human germline genes are used as the source of the variable region framework sequences.

Other methods for humanization include, but are not limited to, a method called “superhumanization” which is described as the direct transfer of CDRs to a human germline framework, a method termed Human String Content (HSC) which is based on a metric of “antibody humanness”, methods based on generation of large libraries of humanized variants (including phage, ribosomal, and yeast display libraries), and methods based on framework region shuffling.

In some instances, the antibody is a human IgG1 antibody. In some instances, the antibody is a human IgG2 antibody. In some instances, the antibody is a human IgG4 antibody. In some instances, the antibody comprises a human kappa light chain constant region. In some instances, the antibody comprises a human lambda light chain constant region.

In some instances, the antibody is an antibody fragment comprising at least one antigen-binding site. In some embodiments, the antibody or antibody fragment is a Fab, Fab′, F (ab′)₂, Fv, scFv, (scFv)₂, single chain antibody, dual variable region antibody, single variable region antibody, linear antibody, diabody, nanobody, or a V region antibody.

In some instances, the antibody is a bispecific antibody. In some instances, the bispecific antibody comprises a first VH and a first VL of a first antibody described herein (e.g., 3P10, Hz3p10, 5F12, or a humanized 5F12) and a second VH and a second VL of a second antibody.

The antibodies described herein can be produced by any suitable method known in the art. Such methods range from direct protein synthesis methods to constructing a DNA sequence encoding polypeptide sequences and expressing those sequences in a suitable host. See, e.g., International Patent Application Publication No. WO 2017/172260, which is incorporated by reference herein in its entirety, for a description of various methods for producing antibodies.

Also provided herein are pharmaceutical compositions comprising an antibody described herein and a pharmaceutically acceptable carrier. A pharmaceutical composition described herein may be used in a method described herein.

In some instances, the pharmaceutical composition comprises an antibody described herein and a pharmaceutically acceptable carrier. In some instances, the pharmaceutical composition comprises: an antibody comprising a VH comprising the VH CDR1, the VH CDR2, and the VH CDR3 of the antibody 3P10 or Hz3P10 (see Table 1 and Table 1A) and a VL comprising the VL CDR1, the VL CDR2, and the VL CDR3 of the antibody 3P10 or Hz3P10 (see Table 1 and Table 1A); and a pharmaceutically acceptable carrier. In some instances, the pharmaceutical composition comprises: an antibody comprising a VH comprising the amino acid sequence set forth in SEQ ID NO: 1982 and a VL comprising the amino acid sequence set forth in SEQ ID NO: 1997; and a pharmaceutically acceptable carrier. In some instances, the pharmaceutical composition comprises: an antibody comprising a heavy chain comprising the amino acid sequence set forth in SEQ ID NO:2010 and a light comprising the amino acid sequence set forth in SEQ ID NO: 2012; and a pharmaceutically acceptable carrier.

Also provided herein is a kit comprising an antibody described herein or a pharmaceutical composition described herein. In some instances, the kit is for use in a method described herein. In some instances, the kit provides instructions for using the antibody or pharmaceutical composition in a method described herein.

### C. METHODS

GDF15 has been shown to induce nausea and vomiting. Further, a genome-wide association study identified GDF15 as being associated with HG and higher levels of circulating GDF15 have been associated with vomiting during pregnancy. Thus, the antibodies described herein (or a pharmaceutical composition comprising the same) are useful in treating, reducing, or preventing nausea, vomiting, or a combination thereof in a human subject (e.g., a human subject suffering from NVP or HG).

Nausea and vomiting of pregnancy (NVP) (also referred to as “morning sickness”) affects up to 70% of pregnant women. NVP typically cases by 14 weeks of gestation. NVP is characterized by: weight loss, adequate food intake most days, nausea and vomiting that is unpleasant but does not limit most essential activities, ability of dietary and lifestyle changes to make symptoms mostly manageable, symptoms that generally case considerably by 14 weeks of gestation, and an ability to complete day-to-day responsibilities (e.g., family responsibilities and employment-related activities) most days, especially after 14 weeks of gestation. NVP may be mild, moderate, or severe.

Hyperemesis gravidarum (HG) is typically considered a more severe form of NVP. HG is estimated to affect up to about 3% and 11% of pregnant women. Hyperemesis gravidarum is characterized by weight loss of at least 5%, inadequate food intake for weeks or months, nausea and vomiting that causes misery and often limits daily activities (including self care), necessary medical treatment for the nausea and vomiting often including intravenous hydration and parenteral nutrition, symptoms that may case or persist until delivery, and difficulty or inability in completing family responsibilities for weeks to months. The International Classification of Diseases characterizes HG as mild or with metabolic disturbance. In some instances, mild HG is vomiting occurring during pregnancy responsive to dietary modification and antiemetic treatment. In some instances, HG with metabolic disturbance is vomiting in pregnancy, not responsive to dietary modification and antiemetic treatment, and associated with electrolyte disturbances and acid-base imbalance. HG typically develops at gestational week 6-8 and usually lasts until gestational week 16-20, but may last until parturition.

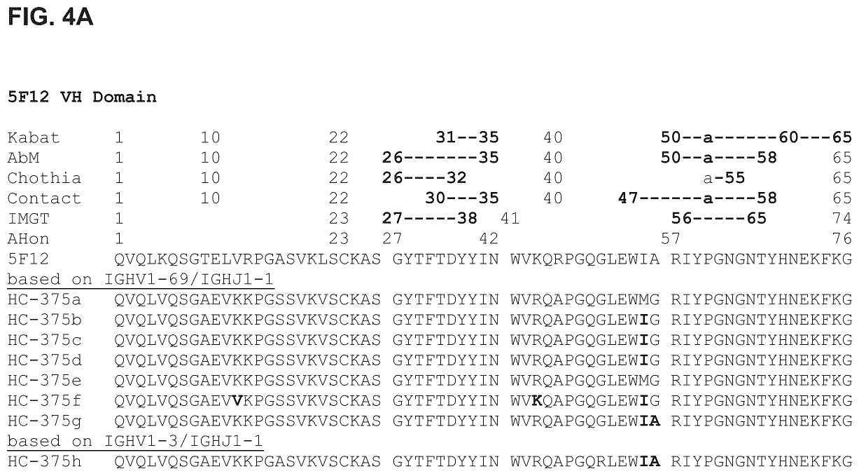

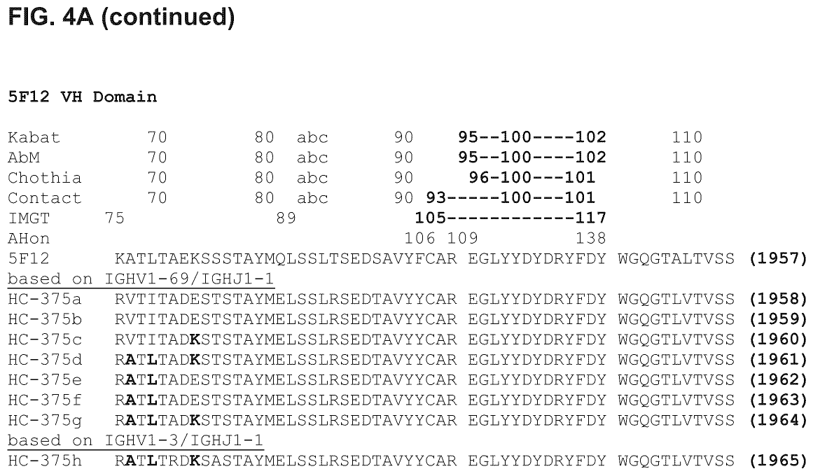

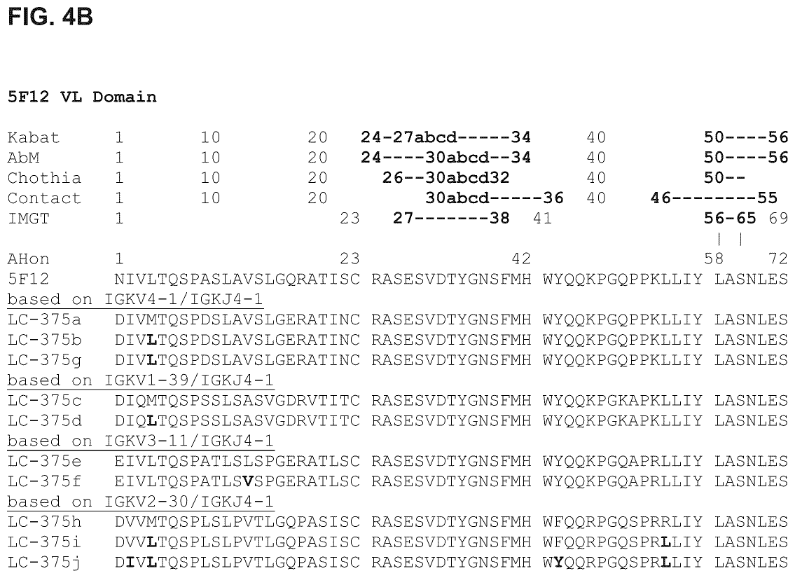

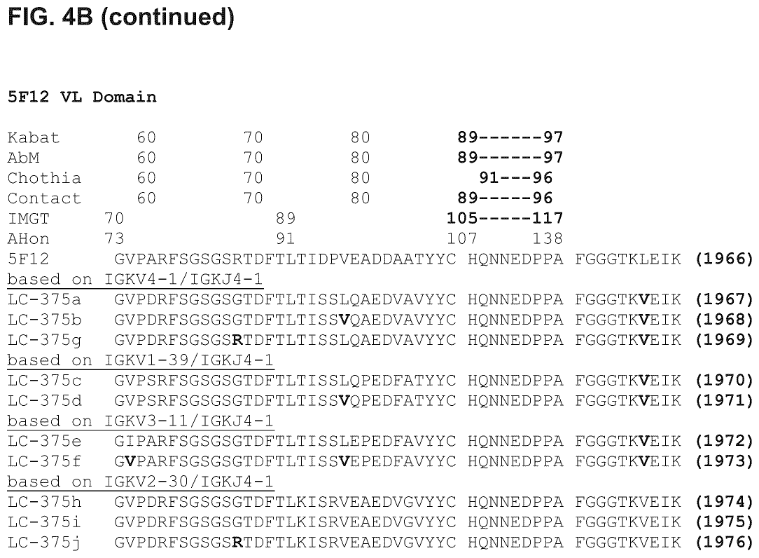

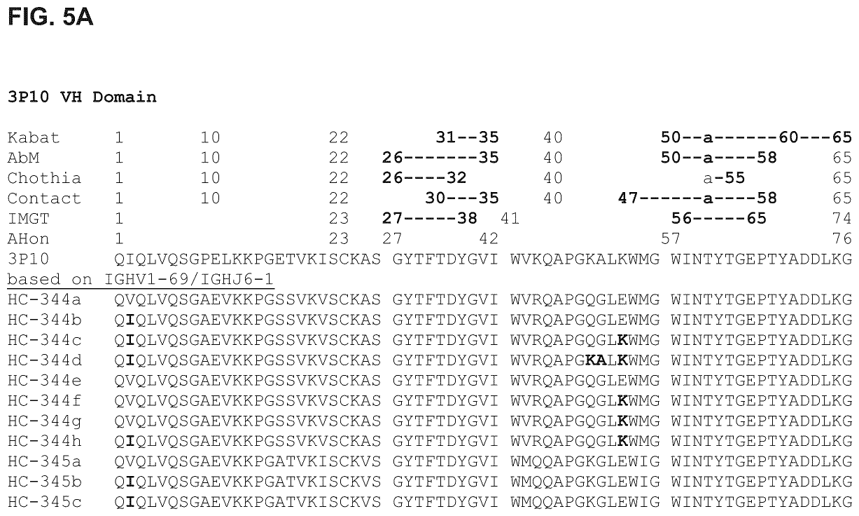

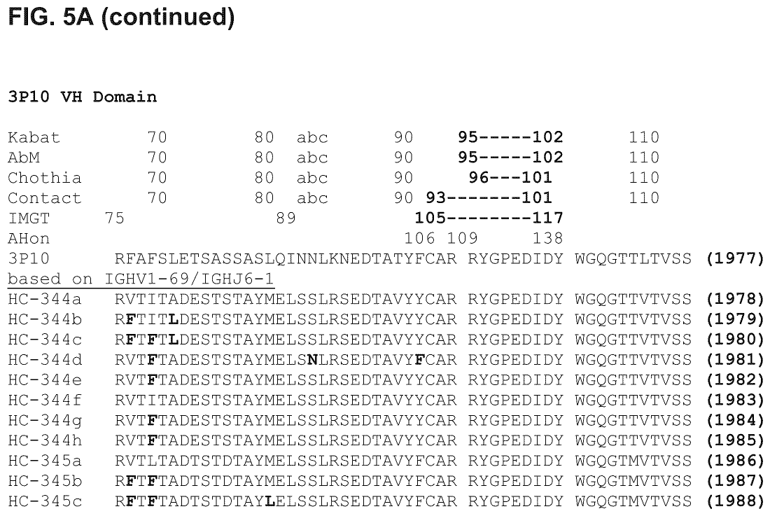

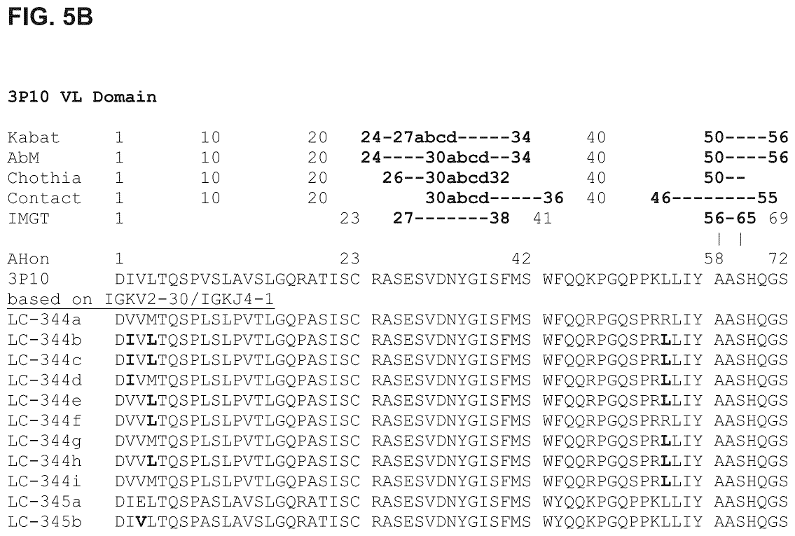

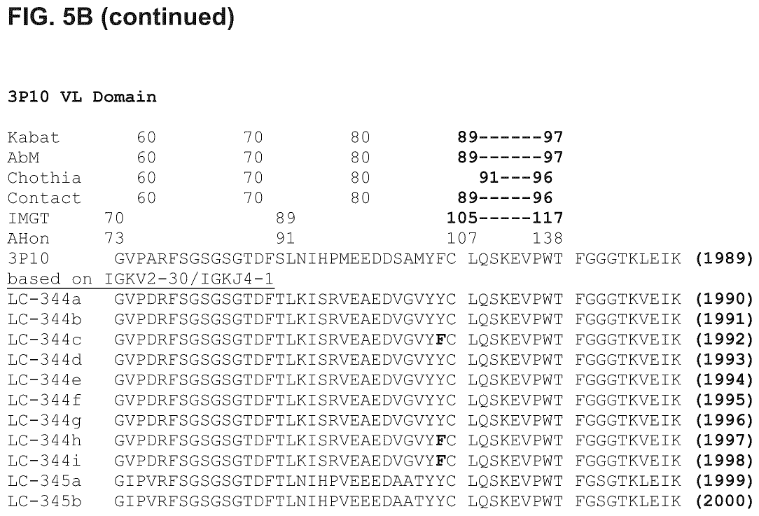

Provided herein is a method for treating nausea, vomiting, or a combination thereof in a human subject in need thereof, the method comprising administering to the human subject a therapeutically effective amount of an antibody that specifically binds human GFRAL (e.g., an antibody described herein) or a pharmaceutical composition comprising the same (e.g., as described herein). In some instances, the human subject does not vomit within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject vomits less than 2, less than 3, less than 4, less than 5, less than 6, less than 7, less than 8, less than 9, less than 10 times, less than 11, less than 12, less than 13, less than 14, or less than 15 times within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject does not retch within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject retches less than 2, less than 3, less than 4, less than 5, less than 6, less than 7, less than 8, less than 9, less than 10 times, less than 11, less than 12, less than 13, less than 14, or less than 15 times within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject does not experience nausea within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject is pregnant (e.g., in the first trimester, in the second trimester, or in the third trimester of pregnancy). In some instances, the human subject has, is suspected of having (e.g., has 1, 2, 3, 4, 5, 6, 7, 8 or more symptoms of HG), or is at risk of developing HG (e.g., is pregnant). In some instances, the human subject has HG. In some instances, the human subject has, is suspected of having (e.g., has 1, 2, 3, 4, 5, 6, 7, 8 or more symptoms of NVP), or is at risk of developing NVP (e.g., is pregnant). In some instances, the human subject has nausea, vomiting, or a combination thereof that is unresponsive to treatment with an antiemetic (e.g., nausea, vomiting, or a combination thereof continues within 1, 2, 3, 4, 5, 6, 7 days of treatment with the antiemetic). In some instances, the antibody is Hz3P10. In some instances, the antibody comprises the CDRs or the VH and VL of antibody Hz3P10 (see Table 1 for the CDRs and Table IA for the VH and VL). In some instances, the antibody is 3P10 or a humanized variant thereof (see, e.g., FIG. 5A and FIG. 5B). In some instances, the antibody is 5F12 or a humanized variant thereof (see, e.g., FIG. 4A and FIG. 4B). In some instances, the antibody comprises the CDRs or the VH and VL of antibody Hz3P10 (see Table 2).

Provided herein is a method for preventing nausea, vomiting, or a combination thereof in a human subject in need thereof, the method comprising administering to the human subject a therapeutically effective amount of an antibody that specifically binds human GFRAL (e.g., an antibody described herein) or a pharmaceutical composition comprising the same (e.g., as described herein). In some instances, the human subject does not vomit within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject does not retch within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject does not experience nausea within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject is pregnant (e.g., in the first trimester, in the second trimester, or in the third trimester of pregnancy). In some instances, the human subject has, is suspected of having (e.g., has 1, 2, 3, 4, 5, 6, 7, 8 or more symptoms of HG), or is at risk of developing HG (e.g., is pregnant). In some instances, the human subject has HG. In some instances, the human subject has, is suspected of having (e.g., has 1, 2, 3, 4, 5, 6, 7, 8 or more symptoms of NVP), or is at risk of developing NVP (e.g., is pregnant). In some instances, the human subject has nausea, vomiting, or a combination thereof that is unresponsive to treatment with an antiemetic (e.g., nausea, vomiting, or a combination thereof continues within 1, 2, 3, 4, 5, 6, 7 days of treatment with the antiemetic). In some instances, the antibody is Hz3P10. In some instances, the antibody comprises the CDRs or the VH and VL of antibody Hz3P10 (see Table 1 for the CDRs and Table 1A for the VH and VL). In some instances, the antibody is 3P10 or a humanized variant thereof (see, e.g., FIG. 5A and FIG. 5B). In some instances, the antibody is 5F12 or a humanized variant thereof (see, e.g., FIG. 4A and FIG. 4B). In some instances, the antibody comprises the CDRs or the VH and VL of antibody Hz3P10 (see Table 2).

Provided herein is a method for treating HG (e.g., mild HG or HG with metabolic disturbance) in a human subject in need thereof, the method comprising administering to the human subject a therapeutically effective amount of an antibody that specifically binds human GFRAL (e.g., an antibody described herein) or a pharmaceutical composition comprising the same (e.g., as described herein). In some instances, the HG is mild HG. In some instances, HG is HG with metabolic disturbance. In some instances, the human subject does not vomit within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject vomits less than 2, less than 3, less than 4, less than 5, less than 6, less than 7, less than 8, less than 9, less than 10 times, less than 11, less than 12, less than 13, less than 14, or less than 15 times within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject does not retch within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject retches less than 2, less than 3, less than 4, less than 5, less than 6, less than 7, less than 8, less than 9, less than 10 times, less than 11, less than 12, less than 13, less than 14, or less than 15 times within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject does not experience nausea within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject is in the first trimester. In some instances, the human subject is in the second trimester. In some instances, the human subject is in the third trimester of pregnancy. In some instances, the human subject has nausea, vomiting, or a combination thereof that is unresponsive to treatment with an antiemetic (e.g., nausea, vomiting, or a combination thereof continues within 1, 2, 3, 4, 5, 6, 7 days of treatment with the antiemetic). In some instances, the antibody is Hz3P10. In some instances, the antibody comprises the CDRs or the VH and VL of antibody Hz3P10 (see Table 1 for the CDRs and Table 1A for the VH and VL). In some instances, the antibody is 3P10 or a humanized variant thereof (see, e.g., FIG. 5A and FIG. 5B). In some instances, the antibody is 5F12 or a humanized variant thereof (see, e.g., FIG. 4A and FIG. 4B). In some instances, the antibody comprises the CDRs or the VH and VL of antibody Hz3P10 (see Table 2).

Also provided herein is a method for treating NVP (e.g., mild NVP, moderate NVP, or severe NVP) in a human subject in need thereof, the method comprising administering to the human subject a therapeutically effective amount of an antibody that specifically binds human GFRAL (e.g., an antibody described herein) or a pharmaceutical composition comprising the same (e.g., as described herein). In some instances, the human subject does not vomit within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject vomits less than 2, less than 3, less than 4, less than 5, less than 6, less than 7, less than 8, less than 9, less than 10 times, less than 11, less than 12, less than 13, less than 14, or less than 15 times within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject does not retch within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject retches less than 2, less than 3, less than 4, less than 5, less than 6, less than 7, less than 8, less than 9, less than 10 times, less than 11, less than 12, less than 13, less than 14, or less than 15 times within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject does not experience nausea within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject is in the first trimester. In some instances, the human subject is in the second trimester. In some instances, the human subject is in the third trimester of pregnancy. In some instances, the human subject has nausea, vomiting, or a combination thereof that is unresponsive to treatment with an antiemetic (e.g., nausea, vomiting, or a combination thereof continues within 1, 2, 3, 4, 5, 6, 7 days of treatment with the antiemetic). In some instances, the antibody is Hz3P10. In some instances, the antibody comprises the CDRs or the VH and VL of antibody Hz3P10 (see Table 1 for the CDRs and Table 1A for the VH and VL). In some instances, the antibody is 3P10 or a humanized variant thereof (see, e.g., FIG. 5A and FIG. 5B). In some instances, the antibody is 5F12 or a humanized variant thereof (see, e.g., FIG. 4A and FIG. 4B). In some instances, the antibody comprises the CDRs or the VH and VL of antibody Hz3P10 (see Table 2).

Also provided herein is method for treating nausea, vomiting, or a combination thereof in a human subject in need thereof, the method comprising administering to the human subject a therapeutically effective amount of an antibody that specifically binds human GFRAL (e.g., an antibody described herein) or a pharmaceutical composition comprising the same (e.g., as described herein), wherein the human subject has unresolved nausea, vomiting, or a combination thereof after treatment with an antiemetic. In some instances, the human subject does not vomit within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject vomits less than 2, less than 3, less than 4, less than 5, less than 6, less than 7, less than 8, less than 9, less than 10 times, less than 11, less than 12, less than 13, less than 14, or less than 15 times within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject does not retch within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject retches less than 2, less than 3, less than 4, less than 5, less than 6, less than 7, less than 8, less than 9, less than 10 times, less than 11, less than 12, less than 13, less than 14, or less than 15 times within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject does not experience nausea within 1, 2, 3, 4, 5, 6, 7, 8, 9, 10, 11, 12, 13, 14, or more days after being administered the antibody (e.g., 1, 2, 3, 4, 5, 6, 7, 8, 9, 10 doses of the antibody). In some instances, the human subject is pregnant (e.g., in the first trimester, in the second trimester, or in the third trimester of pregnancy). In some instances, the human subject has, is suspected of having (e.g., has 1, 2, 3, 4, 5, 6, 7, 8 or more symptoms of HG), or is at risk of developing HG (e.g., is pregnant). In some instances, the human subject has HG. In some instances, the human subject has, is suspected of having (e.g., has 1, 2, 3, 4, 5, 6, 7, 8 or more symptoms of NVP), or is at risk of developing NVP (e.g., is pregnant). In some instances, the human subject has nausea, vomiting, or a combination thereof that is unresponsive to treatment with an antiemetic (e.g., nausea, vomiting, or a combination thereof continues within 1, 2, 3, 4, 5, 6, 7 days of treatment with the antiemetic). In some instances, unresolved vomiting comprises 1, 2, 3, 4, 5, 6, 7, 8, 9, 10, 11, 12, 13, 14, or 15 or more vomiting episodes after treatment with the antiemetic (e.g., within 1, 2, 3, 4, 5, 6, 7, 8, 9, 10, 11, 12, 13, 14 days of 1, 2, 3, 4, 5, 6, 7, 8, 9, 10 doses of the antiemetic). In some instances, unresolved vomiting comprises 1 to 4, 2 to 6, 3 to 7, 4 to 8, 5 to 9, 6 to 10, 7 to 11, 8 to 12, 9 to 13, 10 to 14, or 11 to 15 vomiting episodes after treatment with the antiemetic (e.g., within 1, 2, 3, 4, 5, 6, 7, 8, 9, 10, 11, 12, 13, 14 days of 1, 2, 3, 4, 5, 6, 7, 8, 9, 10 doses of the antiemetic). In some instances, the vomiting episodes are per day. In some instances, the vomiting episodes are an average number of vomiting episodes over the course of several days (e.g., 1, 2, 3, 4, 5, 6, 7 days). In some instances, unresolved vomiting comprises 1, 2, 3, 4, 5, 6, 7, 8, 9, 10, 11, 12, 13, 14, or 15 or more retching episodes per day after treatment with the antiemetic (e.g., within 1, 2, 3, 4, 5, 6, 7, 8, 9, 10, 11, 12, 13, 14 days of 1, 2, 3, 4, 5, 6, 7, 8, 9, 10 doses of the antiemetic). In some instances, unresolved vomiting comprises 1 to 4, 2 to 6, 3 to 7, 4 to 8, 5 to 9, 6 to 10, 7 to 11, 8 to 12, 9 to 13, 10 to 14, or 11 to 15 retching episodes per day after treatment with the antiemetic (e.g., within 1, 2, 3, 4, 5, 6, 7, 8, 9, 10, 11, 12, 13, 14, 15, 16, 17, 18, 19, 20, 21 days of 1, 2, 3, 4, 5, 6, 7, 8, 9, 10 doses of the antiemetic). In some instances, the retching episodes are per day. In some instances, the retching episodes are an average number of retching episodes over the course of several days (e.g., 1, 2, 3, 4, 5, 6, 7 days). In some instances, the antibody is H23P10. In some instances, the antibody comprises the CDRs or the VH and VL of antibody Hz3P10 (see Table 1 for the CDRs and Table 1A for the VH and VL). In some instances, the antibody is 3P10 or a humanized variant thereof (see, e.g., FIG. 5A and FIG. 5B). In some instances, the antibody is 5F12 or a humanized variant thereof (see, e.g., FIG. 4A and FIG. 4B). In some instances, the antibody comprises the CDRs or the VH and VL of antibody Hz3P10 (see Table 2).

Antiemetics are medicines that case nausea or vomiting. Antiemetics are known in the art. Nonlimiting examples of antiemetics include aprepitant, dexamethasone, dimenhydrinate, diphenhydramine, dolasetron, doxylamine, droperidol, granisetron, meclizine, metoclopramide, ondansetron, palonosetron, prochlorperazine, promethazine, rolapitant, and vitamin B6 (pyridoxine). Additional treatments for nausea, vomiting, or a combination thereof include ginger, acupuncture, acupressure, acustimulation, peppermint oil, lavender oil, and lemon oil.

Nausea, vomiting, or a combination thereof (e.g., as in NVP or HG), is also associated with numerous sequela, including hyponatremia, hypovolemia, ketonuria, electrolyte abnormalities, vitamin deficiencies, and weight loss. Thus, the antibodies described herein are also useful in treating a sequelae of nausea, vomiting, or a combination thereof, such as hyponatremia, hypovolemia, ketonuria, electrolyte abnormalities, vitamin deficiencies, and weight loss in a human subject suffering from nausea, vomiting, or a combination thereof (e.g., a human subject suffering from NVP or HG).

Thus, also provided herein is a method for treating hypovolemia in a human subject in need thereof, the method comprising administering to the human subject a therapeutically effective amount of an antibody that specifically binds human GFRAL (e.g., an antibody described herein) or a pharmaceutical composition comprising the same (e.g., as described herein), wherein the human subject is suffering from nausea, vomiting, or a combination thereof. Also provided herein is a method for treating weight loss in a human subject in need thereof, the method comprising administering to the human subject a therapeutically effective amount of an antibody that specifically binds human GFRAL (e.g., an antibody described herein) or a pharmaceutical composition comprising the same (e.g., as described herein), wherein the human subject is pregnant, and wherein the human subject is suffering from nausea, vomiting, or a combination thereof (e.g., is suffering from HG or NVP). Also provided herein is a method for treating electrolyte abnormalities in a human subject in need thereof, the method comprising administering to the human subject a therapeutically effective amount of an antibody that specifically binds human GFRAL (e.g., an antibody described herein) or a pharmaceutical composition comprising the same (e.g., as described herein), wherein the human subject is suffering from nausea, vomiting, or a combination thereof (e.g., is suffering from HG or NVP). Also provided herein is a method for treating ketonuria in a human subject in need thereof, the method comprising administering to the human subject a therapeutically effective amount of an antibody that specifically binds human GFRAL (e.g., an antibody described herein) or a pharmaceutical composition comprising the same (e.g., as described herein), wherein the human subject is suffering from nausea, vomiting, or a combination thereof (e.g., HG or NVP).

In some instances of a method described herein, the human subject has been diagnosed with HG. In some instances of a method described herein, the human subject is at risk for developing HG. In some instances of a method described herein, the human subject has one or more (e.g., 1, 2, 3, 4, 5, 6, 7, 8) symptoms of HG.

In some instances of a method described herein, the human subject has been diagnosed with NVP. In some instances of a method described herein, the human subject is at risk for developing NVP. In some instances of a method described herein, the human subject has one or more (e.g., 1, 2, 3, 4, 5, 6, 7, 8) symptoms of NVP.

In some instances, the human subject is in the first trimester of pregnancy. In some instances, the human subject is in the second trimester of pregnancy. In some instances, the human subject is in the third trimester of pregnancy. In some instances, the human subject is 1 to 12 weeks pregnant. In some instances, the human subject is 12 to 24 weeks pregnant. In some instances, the human subject is 24 to 40 weeks pregnant. In some instances, the human subject is in week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 29, week 30, week 31, week 32, week 33, week 34, week 35, week 36, week 37, week 38, week 39, or week 40 of pregnancy.

In some instances, a biological sample obtained from the human subject has an elevated level of GDF15. In some instances, the biological sample is obtained from the human subject at baseline (i.e., prior to administration of the antibody, e.g., within 1, 2, 3, 4, 5, 6, 7, 8, 9, 10 days prior to administration of the antibody). In some instances, the biological sample is a blood sample, a plasma sample, or a serum sample. In some instances, the elevated level of GDF15 is as compared to a control level of GDF15 in a human or a panel of humans (e.g., 2, 3, 4, 5, 10, 15, 20, 25, or more humans) not suffering from NVP, HG, nausea, or vomiting or is/are not pregnant. In some instances, the level of GDF15 is an mRNA level of GDF15. In some instances, the level of GDF15 is a protein level of GDF15. In some instances, the biological sample (e.g., serum) obtained from the human subject has a level of GDF15 that is at least 500 μg/mL (e.g., at least 500 μg/mL, at least 750 μg/mL, at least 1,000 μg/mL, at least 1,500 μg/mL, at least 2,000 μg/mL, at least 3,000 μg/mL, at least 4,000 μg/mL, at least 5,000 μg/mL, at least 6,000 μg/mL, at least 7,000 μg/mL, at least 8,000 μg/mL, at least 9,000 μg/mL, at least 10,000 μg/mL, at least 15,000 μg/mL, at least 20,000 μg/mL, at least 25,000 μg/mL, at least 30,000 μg/mL, at least 35,000 μg/mL, at least 40,000 μg/mL, at least 45,000 μg/mL, or at least 50,000 μg/mL). In some instances, the biological sample (e.g., serum) obtained from the human subject has a level of GDF15 that is between 1,000 and 60,000 μg/mL, inclusive. In some instances, the biological sample (e.g., serum) obtained from the human subject has a level of GDF15 that is between 2,000 and 60,000 μg/mL, inclusive. In some instances, the biological sample (e.g., serum) obtained from the human subject has a level of GDF15 that is between 5,000 and 60,000 μg/mL, inclusive. In some instances, the biological sample (e.g., serum) obtained from the human subject has a level of GDF15 that is between 8,000 and 15,000 μg/mL, inclusive.

In some instances, the human subject is unresponsive to an antiemetic. In some instances, the antiemetic is promethazine or ondansetron. In some instances, the human subject has been administered one or more (e.g., 1, 2, 3, 4, 5, 6, 7, 8, 9, 10) doses of the antiemetic but still suffers from nausea, vomiting, or a combination thereof.

In some instances, the human subject does not respond to conservative management of nausea and vomiting during pregnancy. Conservative management includes diet modification, emotional support, ginger, acupressure, or combinations thereof. In some instances, the human subject does not respond to intravenous fluids, an antiemetic, corticosteroids, or parenteral nutrition, or combinations thereof.

The Pregnancy-Unique Quantification of Emesis (PUQE) questionnaire may be used to measure the severity of NVP or HG (see Koren et al., 2002, Am. J. Obstet Gynecol., 186 (5: Suppl Understanding): S228-31, which is incorporated by reference herein in it its entirety), which is based on the number of daily vomiting episodes, the length of nausea per day in hours, and the number of retching episodes. In some instances, the human subject has a PUQE score of 4 to 6 at baseline (i.e., prior to administration of the antibody, e.g., within 1, 2, 3, 4, 5, 6, 7, 8, 9, 10 days prior to administration of the antibody). In some instances, the human subject has a PUQE score of 7 to 12 at baseline. In some instances, the human subject has a PUQE score of 13 or higher at baseline.

In some instances, the human subject vomits 1, 2, 3, 4, 5, 6, 7, 8, 9, 10, 11, 12, 13 or more times per day at baseline (i.e., prior to administration of the antibody, e.g., within 1, 2, 3, 4, 5, 6, 7, 8, 9, 10 days prior to administration of the antibody). In some instances, the human subject vomits 1-2 times per day at baseline. In some instances, the human subject vomits 3-5 times per day at baseline. In some instances, the human subject vomits 6-8 times per day at baseline. In some instances, the human subject vomits 9-12 times per day at baseline. In some instances, the human subject vomits 13 or more times per day at baseline. In some instances, the human subject retches 1, 2, 3, 4, 5, 6, 7, 8, 9, 10, 11, 12, 13 or more times per day at baseline (i.e., prior to administration of the antibody, e.g., within 1, 2, 3, 4, 5, 6, 7, 8, 9, 10 days prior to administration of the antibody).

In some instances, the human subject retches 1, 2, 3, 4, 5, 6, 7, 8, 9, 10, 11, 12, 13 or more times per day at baseline (i.e., prior to administration of the antibody, e.g., within 1, 2, 3, 4, 5, 6, 7, 8, 9, 10 days prior to administration of the antibody). In some instances, the human subject retches 1-2 times per day at baseline. In some instances, the human subject retches 3-5 times per day at baseline. In some instances, the human subject retches 6-8 times per day at baseline. In some instances, the human subject retches 9-12 times per day at baseline. In some instances, the human subject retches 13 or more times per day at baseline.

In some instances, the human subject loses at least 1%, at least 2%, at least 3%, at least 4%, or at least 5% of weight over the course of several (e.g., 2, 3, 4, 5, 6, 7, 8, 9, 10) days prior to administration of the antibody (e.g., within 1, 2, 3, 4, 5, 6, 7, 8, 9, 10 days prior to administration of the antibody). In some instances, the human subject loses 1%-10%, 1% to 7%, 1% to 5%, 3%-10%, 3% to 7%, 3% to 5%, or 5% to 10% of weight over the course of several (e.g., 2, 3, 4, 5, 6, 7, 8, 9, 10) days prior to administration of the antibody (e.g., within 1, 2, 3, 4, 5, 6, 7, 8, 9, 10 days prior to administration of the antibody).

In some instances, the method comprises administering to the human subject a single dose of the therapeutically effective amount of the antibody. In some instances, the single dose is administered during the first trimester of pregnancy. In some instances, the single dose is administered during the second trimester of pregnancy. In some instances, the single dose is administered during the third trimester of pregnancy. In some instances, the single dose is administered during the first or second trimester of pregnancy. In some instances, the single dose is administered during the second or third trimester of pregnancy. In some instances, the single dose is administered during week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 29, week 30, week 31, week 32, week 33, week 34, week 35, week 36, week 37, week 38, week 39, or week 40 of pregnancy.

In some instances, the method comprises administering to the human subject at least 2, at least 3, at least 4, at least 5, at least 6, at least 7 at least 8, at least 9, at least 10, at least 11, at least 12 doses, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more doses of the therapeutically effective amount of the antibody. In some instances, the dose is administered once every 1 week, once every 2 weeks, once every 3 weeks, once every 4 weeks, once every 5 weeks, once every 6 weeks, once every 7 weeks, once every 8 weeks, once every 9 weeks, once every 10 weeks. In some instances, the dose is administered once every about 4 weeks. In some instances, the dose is administered once every about 4 weeks. In some instances, the dose is administered once every about 3 weeks. In some instances, the dose is administered once every 3 weeks. In some instances, the first dose is administered during the first trimester of pregnancy. In some instances, the first dose is administered during the second trimester of pregnancy. In some instances, the first dose is administered during the third trimester of pregnancy. In some instances, the first dose is administered during the first or second trimester of pregnancy. In some instances, the first dose is administered during the second or third trimester of pregnancy. In some instances, the first dose is administered during week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 29, week 30, week 31, week 32, week 33, week 34, week 35, week 36, week 37, week 38, week 39, or week 40 of pregnancy. In some instances, the last dose is administered during the first trimester of pregnancy. In some instances, the last dose is administered during the second trimester of pregnancy. In some instances, the last dose is administered during the third trimester of pregnancy. In some instances, the last dose is administered during the first or second trimester of pregnancy. In some instances, the last dose is administered during the second or third trimester of pregnancy. In some instances, the dose is administered during week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 29, week 30, week 31, week 32, week 33, week 34, week 35, week 36, week 37, week 38, week 39, or week 40 of pregnancy.

In some instances, the therapeutically effective amount of the antibody is 1 mg to 150 mg, 10 mg to 150 mg, 25 mg to 150 mg, 50 mg to 150 mg, 75 mg to 125 mg, 90 mg to 110 mg, 95 mg to 105 mg, 10 mg to 50 mg, 20 mg to 40 mg and 25 mg to 35 mg. In some instances, the therapeutically effective amount of the dose is about 10 mg, 20 mg, 30 mg, 40 mg, 50 mg, 60 mg, 70 mg, 80 mg, 90 mg, 100 mg, 110 mg, 120 mg, or more. In some instances, the therapeutically effective amount of the dose is about 100 mg. In some instances, the therapeutically effective amount of the dose is about 75 mg. In some instances, the therapeutically effective amount of the dose is about 30 mg. In some instances, the therapeutically effective amount of the dose is 100 mg. In some instances, the therapeutically effective amount of the dose is 75 mg. In some instances, the therapeutically effective amount of the dose is 30 mg.

In some instances, the therapeutically effective amount of the antibody is administered to the human subject subcutaneously.

In some instances, the therapeutically effective amount of the antibody is administered to the human subject intravenously.

In some instances, the human subject does not lose more than 1%, more than 2%, more than 3%, more than 4%, or more than 5% of weight over the course of several (e.g., 2, 3, 4, 5, 6, 7, 8, 9, 10) days after administration of the antibody (e.g., within 1, 2, 3, 4, 5, 6, 7, 8, 9, 10, 11, 12, 13, 14, 15, 16, 17, 18, 19, 20, 21 days after administration of 1, 2, 3, 4, 5, or more doses of the antibody). In some instances, the lack of weight loss is observed until the third trimester. In some instances, the lack of weight loss is observed until the end of the pregnancy. In some instances, the lack of weight loss is relative to the subject's weight immediately prior to therapy. In other instances, the lack of weight loss is relative to the expected weight and/or weight gain of the subject at that stage of gestation in view of the fact that subjects normally progressively gain weight through gestation. In other instances, the lack of weight loss is determined as no change in the rate of weight gain following treating, relative to normal subjects that have not received therapy.

In some instances, a method described herein reduces the human subject's PUQE score by at least 0.5, at least 0.75, at least 1.0, at least 1.25, at least 1.5, at least 1.75, at least 2, at least 2.25, at least 2.5, at least 2.75, at least 3, at least 3.25, at least 3.5, at least 3.75, at least 4, at least 4.25, at least 4.5, at least 4.75, at least 5, at least 5.25, at least 5.5, at least 5.75, at least 6, at least 6.25, at least 6.5, at least 6.75, at least 7, at least 7.25, at least 7.5, at least 7.75, or at least 8 compared to baseline (i.e., prior to administration of the antibody, e.g., within 1, 2, 3, 4, 5, 6, 7, 8, 9, 10 days prior to administration of the antibody). In some instances, a method described herein reduces the human subject's PUQE score to less than 10, less than 9, less than 8, less than 7, less than 6, or less than 5 (e.g., within 1, 2, 3, 4, 5, 6, 7, 8, 9, 10, 11, 12, 13, 14, 15, 16, 17, 18, 19, 20, 21 days after administration of 1, 2, 3, 4, 5, or more doses of the antibody). In some instances, the reduction in the human subject's PUQE score is observed until the third trimester. In some instances, the reduction in the human subject's PUQE score is observed until the end of the pregnancy.

In some instances, a method described herein reduces the number of vomiting episodes per day by 1, 2, 3, 4, 5, 6, 7, 8, 9, 10, 11, 12, 13, or more compared to baseline (i.e., prior to administration of the antibody, e.g., within 1, 2, 3, 4, 5, 6, 7, 8, 9, 10 days prior to administration of the antibody). In some instances, a method described herein reduces the number of vomiting episodes per day to less than 13, less than 12, less than 11, less than 10, less than 9, less than 8, less than 7, less than 6, less than 5, less than 4, less than 3, less than 2, 1, or 0 (e.g., within 1, 2, 3, 4, 5, 6, 7, 8, 9, 10, 11, 12, 13, 14, 15, 16, 17, 18, 19, 20, 21 days after administration of 1, 2, 3, 4, 5, or more doses of the antibody). In some instances, the reduction in the number of vomiting episodes is an average number of vomiting episodes (e.g., over 2, 3, 4, 5, 6, 7, 8, 9, 10 days). In some instances, a reduction in number of vomiting episodes is observed for at least 1, at least 2, at least 3, at least 4, at least 5, at least 6, at least 7, at least 8, at least 9, at least 10, at least 11, at least 12, at least 13, at least 14, at or least 15 weeks. In some instances, the reduction in number of vomiting episodes is observed until the third trimester. In some instances, the reduction in number of vomiting episodes is observed until the end of the pregnancy.

In some instances, a method described herein reduces the number of retching episodes per day by 1, 2, 3, 4, 5, 6, 7, 8, 9, 10, 11, 12, 13, or more compared to baseline (i.e., prior to administration of the antibody, e.g., within 1, 2, 3, 4, 5, 6, 7, 8, 9, 10 days prior to administration of the antibody). In some instances, a method described herein reduces the number of retching episodes per day to less than 13, less than 12, less than 11, less than 10, less than 9, less than 8, less than 7, less than 6, less than 5, less than 4, less than 3, less than 2, 1, or 0 (e.g., within 1, 2, 3, 4, 5, 6, 7, 8, 9, 10, 11, 12, 13, 14, 15, 16, 17, 18, 19, 20, 21 days after administration of 1, 2, 3, 4, 5, or more doses of the antibody). In some instances, the reduction in the number of retching episodes is an average number of retching episodes (e.g., over 2, 3, 4, 5, 6, 7, 8, 9, 10 days). In some instances, a reduction in number of retching episodes is observed for at least 1, at least 2, at least 3, at least 4, at least 5, at least 6, at least 7, at least 8, at least 9, at least 10, at least 11, at least 12, at least 13, at least 14, at or least 15 weeks. In some instances, the reduction in number of retching episodes is observed until the third trimester. In some instances, the reduction in number of retching episodes is observed until the end of the pregnancy.

In some instances, a method described herein reduces the severity of nausea to moderate, to mild, or to no nausea. Methods of scoring nausea are known in the art. For instance, nausea may be scored on a scale of 0 to 5, 0 being no nausea, 1 to 2 being mild nausea, 3-4 being moderate nausea, and 5 being severe nausea. In some instances, the reduction in the severity of nausea occurs within 1, 2, 3, 4, 5, 6, 7, 8, 9, 10, 11, 12, 13, 14, 15, 16, 17, 18, 19, 20, 21 days after administration of 1, 2, 3, 4, 5, or more doses of the antibody. In some instances, the reduction in the severity of nausea is observed until the third trimester. In some instances, the reduction in the severity of nausea is observed until the end of the pregnancy.

In some instances, a method described herein further comprises administering to the subject an antiemetic or other therapy for nausea, vomiting, or a combination thereof. Nonlimiting examples of antiemetics include aprepitant, dexamethasone, dimenhydrinate, diphenhydramine, dolasetron, doxylamine (e.g., doxylamine succinate), droperidol, granisetron, meclizine, metoclopramide, ondansetron, palonosetron, prochlorperazine, chlorpromazine, promethazine, rolapitant, and vitamin B6 (pyridoxine such as pyridoxine hydrochloride). Additional treatments for nausea, vomiting, or a combination thereof include ginger, acupuncture, acupressure, acustimulation, and essential oils (e.g., peppermint, lavender, and lemon). In some cases, the additional treatment is a dual release formulation of doxylamine and pyridoxine.

In some instances, the antibody comprises (i) a VH comprising the VH CDR1, the VH CDR2, the VH CDR3 from the amino acid sequence set forth in SEQ ID NO:3 or 1982; and (ii) a VL comprising the VL CDR1, the VL CDR2, and the VL CDR3 from the amino acid sequence set forth in SEQ ID NO:4 or 1997. In some instances, the antibody comprises: (i) a VH comprising a VH CDR1 comprising the amino acid sequence set forth in SEQ ID NO:46, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the antibody comprises: (i) a VH comprising a VH CDR1 comprising the amino acid sequence set forth in SEQ ID NO:48, a VH CDR2 comprising the amino acid sequence set forth in SEQ ID NO:137, and a VH CDR3 comprising the amino acid sequence set forth in SEQ ID NO:225; and (ii) a VL comprising a VL CDR1 comprising the amino acid sequence set forth in SEQ ID NO:301, a VL CDR2 comprising the amino acid sequence set forth in SEQ ID NO:376, and a VL CDR3 comprising the amino acid sequence set forth in SEQ ID NO:426. In some instances, the antibody comprises: (i) a VH comprising an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:1982; and (ii) a VL comprising an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:1997. In some instances, the antibody comprises a VH comprising the amino acid sequence set forth in SEQ ID NO:1982. In some instances, the antibody comprises a VL comprising the amino acid sequence set forth in SEQ ID NO:1997. In some instances, the antibody comprises: (i) a VH comprising the amino acid sequence set forth in SEQ ID NO:1982; and (ii) a VL comprising the amino acid sequence set forth in SEQ ID NO:1997. In some instances, the antibody comprises a heavy chain comprising an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:2010. In some instances, the antibody comprises a light chain comprising an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:2012. In some instances, the antibody comprises: (i) a heavy chain comprising an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:2010; and (ii) a light chain comprising an amino acid sequence having at least at least 75%, at least 80%, at least 85%, at least 90%, at least 95%, at least 96%, at least 97%, at least 98%, at least 99%, or 100% sequence identity to the amino acid sequence set forth in SEQ ID NO:2012. In some instances, the antibody comprises a heavy chain comprising the amino acid sequence set forth in SEQ ID NO:2010. In some instances, the antibody comprises a heavy chain consisting of the amino acid sequence set forth in SEQ ID NO:2010. In some instances, the antibody comprises a light chain comprising the amino acid sequence set forth in SEQ ID NO:2012. In some instances, the antibody comprises a light chain consisting of the amino acid sequence set forth in SEQ ID NO:2012. In some instances, the antibody comprises: (i) a heavy chain comprising the amino acid sequence set forth in SEQ ID NO:2010; and (ii) a light chain comprising the amino acid sequence set forth in SEQ ID NO:2012. In some instances, the antibody comprises: (i) a heavy chain consisting of the amino acid sequence set forth in SEQ ID NO:2010; and (ii) a light chain consisting of the amino acid sequence set forth in SEQ ID NO:2012. In some instances, the therapeutically effective amount of the antibody is about 30 mg. In some instances, the therapeutically effective amount of the antibody is 30 mg. In some instances, the therapeutically effective amount of the antibody is about 100 mg. In some instances, the therapeutically effective amount of the antibody is 100 mg. In some instances, the antibody is administered once about every 2 weeks, 3 weeks., 4 weeks, 5 weeks, 6 weeks, 7 weeks, or 8 weeks, or even only once, twice, or thrice during pregnancy. In some instances, the antibody is administered once every 3 weeks or 4 weeks. In some instances, the therapeutically effective amount of the is about 30 mg administered once about every 3 weeks or 4 weeks. In some instances, the therapeutically effective amount of the antibody is 30 mg administered once every 3 weeks or 4 weeks. In some instances, the therapeutically effective amount of the antibody is about 100 mg administered once about every 3 weeks or 4 weeks. In some instances, the therapeutically effective amount of the antibody is 100 mg administered once every 3 weeks or 4 weeks. In some instances, the antibody is administered subcutaneously. In some instances, the antibody is administered subcutaneously at a dose of 30 mg once every 3 weeks or 4 weeks. In some instances, the antibody is administered subcutaneously at a dose of 100 mg once every 3 weeks or four weeks. In some instances, the therapeutically effective amount is 30 mg or 100 mg and is administered once, twice, or thrice throughout pregnancy. In some instances, the antibody is administered on an as-needed basis to control symptoms.

In some instances, the antibody comprises the CDRs, the VH and VL, or the heavy chain and light chain of antibody Hz3P10 (see Table 1A). In some instances, the method comprises administering a single dose of the antibody. In some instances, the single dose is administered during the first trimester. In some instances, the single dose is administered during the second trimester. In some instances, the single dose is administered during the third trimester. In some instances, the single dose is about 30 mg. In some instances, the single dose is about 100 mg. In some instances, the single dose is 30 mg. In some instances, the single dose is 100 mg. In some instances, the administering is subcutaneously.

In some instances, the antibody comprises the CDRs, the VH and VL, or the heavy chain and light chain of antibody Hz3P10 (see Table 1A). In some instances, the method comprises administering more than one (e.g., 1, 2, 3, 4, 5, 6, 7, 8, 9, 10, 11, 12, 13, 14, 15, or more) dose of the antibody. In some instances, the first dose is administered during the first trimester. In some instances, the first dose is administered during the second trimester. In some instances, the first dose is administered during the third trimester. In some instances, the single dose is 30 mg. In some instances, the single dose is 100 mg. In some instances, the single dose is about 30 mg. In some instances, the single dose is about 100 mg. In some instances, the administering is subcutaneously.

In some instances, the antibody comprises the CDRs or the VH and VL antibody 5F12 (see Table 1A) or a humanized version thereof (see, e.g., FIG. 4A and FIG. 4B). In some instances, the method comprises administering a single dose of the antibody. In some instances, the single dose is administered during the first trimester. In some instances, the single dose is administered during the second trimester. In some instances, the single dose is administered during the third trimester. In some instances, the single dose is about 30 mg. In some instances, the single dose is about 100 mg. In some instances, the single dose is 30 mg. In some instances, the single dose is 100 mg. In some instances, the administering is subcutaneously.

In some instances, the antibody comprises the CDRs or the VH and VL antibody 5F12 (see Table 1A) or a humanized version thereof (see, e.g., FIG. 4A and FIG. 4B). In some instances, the method comprises administering more than one (e.g., 1, 2, 3, 4, 5, 6, 7, 8, 9, or 10) dose of the antibody. In some instances, the first dose is administered during the first trimester. In some instances, the first dose is administered during the second trimester. In some instances, the first dose is administered during the third trimester. In some instances, the dose is about 30 mg. In some instances, the dose is about 75 mg. In some instances, the single dose is about 100 mg. In some instances, the single dose is 30 mg. In some instances, the single dose is 75 mg. In some instances, the single dose is 100 mg. In some instances, the administering is subcutaneously. In some instances, the administering is intravenously.

### VI. EXAMPLES

Multiple studies in mice and rats have demonstrated that the anti-GFRAL antibody hz3P10 can ameliorate the impact of GDF15-induced weight loss. Weight loss in rodents is used as a surrogate for nausea and vomiting in humans since rodents are incapable of vomiting. Serum levels of GDF15 were increased by direct administration of GDF15-Fc fusion protein by use of tumor models which secrete GDF15, by use of an adeno-associated virus, or by chemotherapy administration stress-induction of GDF15 increases. In all models, hz3P10 at doses ≥1 mg/kg ameliorated GDF15-induced weight loss.

### Example 1: Body Weight Change in Mice Administered Subcutaneous Hz3P10 Following Induction of Weight Loss by GDF15-Fc Fusion Protein

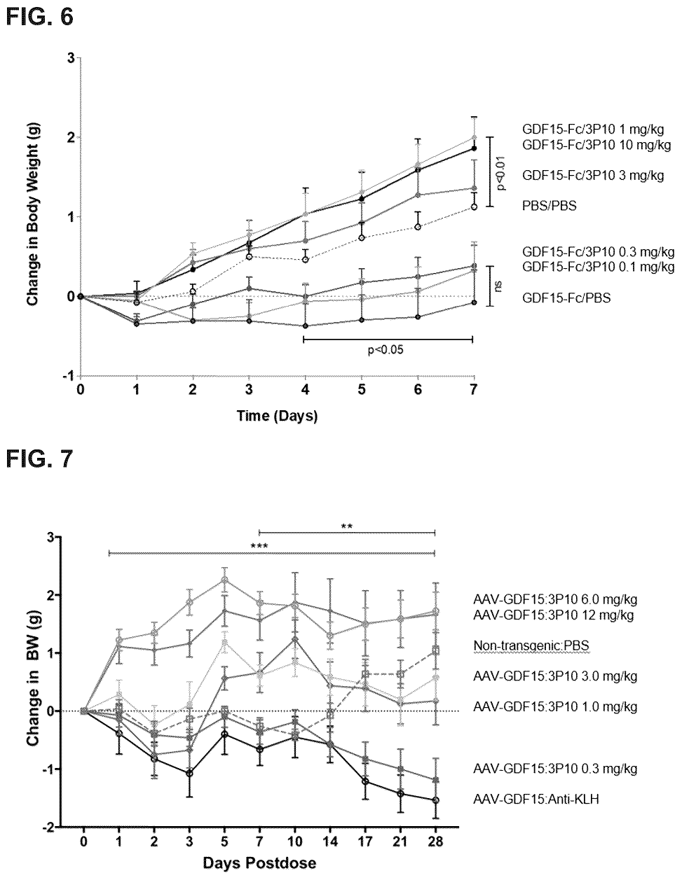

This study evaluated the efficacy and identified no-effect and saturable doses of subcutaneous (SC) 3P10 on blocking GDF15-induced weight loss in obese mice (male, n=8/dose group, ˜19 weeks old at study start). Mice were administered a single dose of a GDF15-Fc fusion protein SC at 0 (phosphate buffered saline [PBS] control) or 0.1 mg/kg to induce weight loss. Two days later, mice receiving the GDF15-Fc fusion protein were randomized into groups to receive a single SC injection of hz3P10 at 0 (PBS control), 0.1, 0.3, 1, 3 or 10 mg/kg. Body weights were evaluated daily for 1 week post hz3P10 injection. As shown in FIG. 6, mice that received PBS instead of GDF15-Fc fusion protein gained weight steadily throughout the study as did mice that received the 0.1 mg/kg GDF15-Fc fusion protein followed 2 days later by hz3P10 doses of 1, 3 or 10 mg/kg. Mice that received GDF15-Fc fusion protein followed by 0, 0.1 or 0.3 mg/kg 3P10 lost or did not gain weight, compared to the controls. In sum, a single SC dose of 3P10 at ≥1 mg/kg prevented GDF15-induced weight loss in obese mice.

### Example 2: Dose Response of Hz3P10 in Reversal of GDF15-Induced Weight Loss Over 28 Days in Mice

This study evaluated the activity of single SC doses of hz3P10 in a GDF15-induced body weight reduction model in lean mice over a 28-day period to identify the no- and maximally efficacious doses. C57BL/6J mice (male, n=8/dose group, ˜18 weeks old at study start) were administered either placebo (PBS) or an adeno-associated virus (AAV) carrying GDF15 DNA (AAV-GDF15) to induce weight loss via continuous high levels of GDF15 production. Serum GDF15 and body weights were measured throughout the study. Two weeks post AAV-GDF15 induction, mice with GDF15 levels greater than 1 ng/ml were selected and randomized to receive single SC doses of hz3P10 at 0 (isotype control antibody), 0.3, 1, 3, 6 or 12 mg/kg. Following hz3P10 administration, mice in the control group and the 0.3 mg/kg 3P10 group continued to lose weight (FIG. 7). By the end of study, doses of 1 and 3 mg/kg resulted in weight gain similar to a non-transgenic control group while 6 and 12 mg/kg increased weight gain above that of non-transgenic controls. Mice receiving ≥3 mg/kg hz3P10 also had increased food consumption compared to the AAV-GDF15 control group. In sum, a single SC dose of hz3P10 reversed GDF15-induced reductions in body weight in lean mice over 28 days. In mice, the no-effect dose of hz3P10 in blocking GDF15-induced weight loss was 0.3 mg/kg while 6 mg/kg was fully efficacious.

### Example 3: Dose Response of Hz3P10 in Reversal of HT1080 Tumor-Induced Weight Loss in SCID Mice

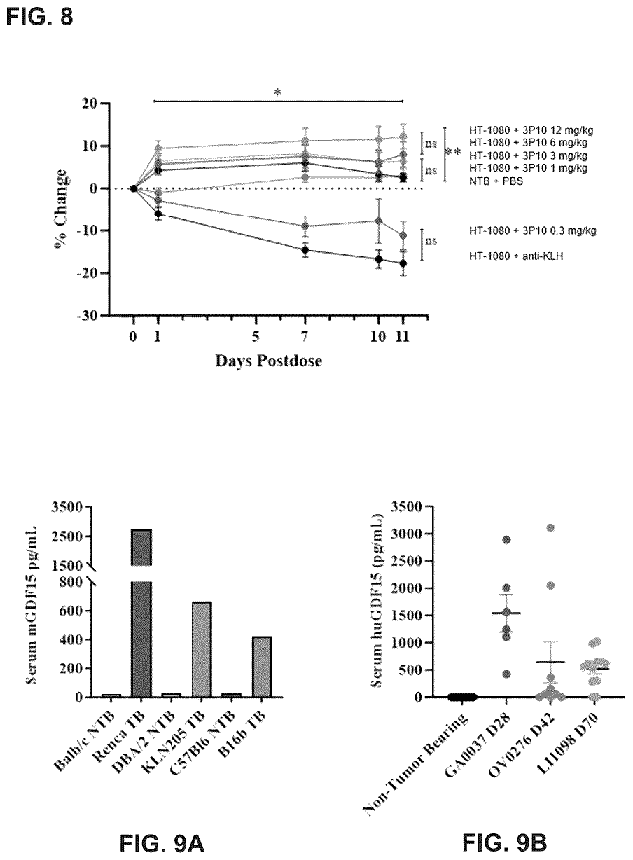

This study evaluated the efficacy and potency of a single SC dose of hz3P10 at inhibiting GDF15-induced reductions in body weight in severe combined immunodeficiency (SCID) HT1080 xenograft mice and identified no- and maximally efficacious doses. Institute of Cancer Research (ICR) SCID mice (female, n=7/dose group, ˜7 weeks old at study start) were implanted SC with HT1080 tumor cells, which produce hGDF15, and tumors were allowed to grow for 2 weeks; a control group remained uninoculated. Mice were then randomized by weight loss into 0 (anti-keyhole limpet hemocyanin [KLH] control), 0.3, 1, 3, 6 and 13 mg/kg 3P10 SC dose groups. Following single dose administration, mice were assessed for an additional 11 days, with body weights and food consumption measured throughout. GDF15 serum levels were measured at termination. Non-tumor bearing controls had hGDF15 levels that were not measurable, while a separate cohort of tumor bearing mice had hGDF15 levels ranging from 740 to 18,180 pg/mL (FIG. 8).

### Example 4: 3P10 and Hz3P10 Efficacy in Inhibiting Tumor-Induced Weight Loss in PDX and Syngeneic Mouse Tumor Models

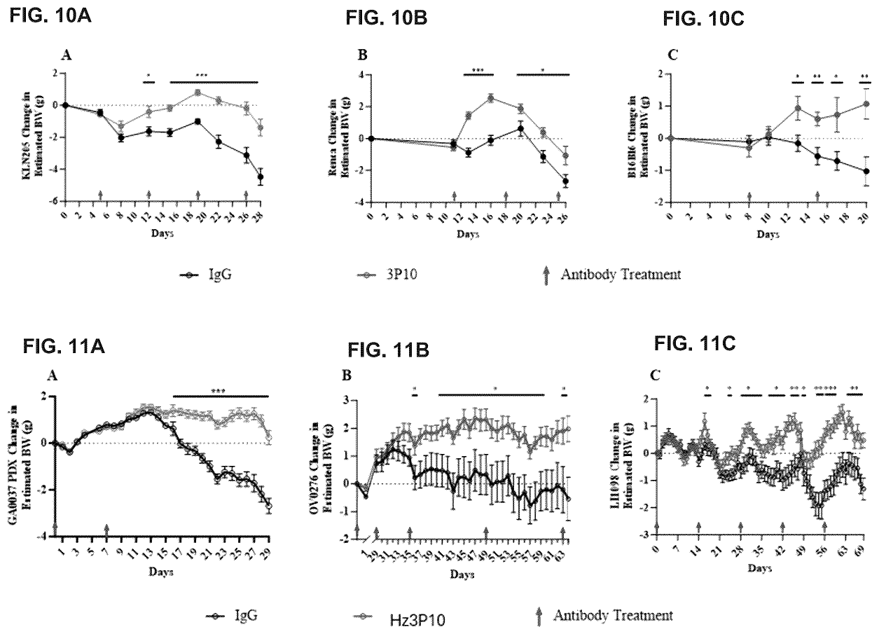

This study assessed the efficacy of hz3P10 or 3P10, the hz3P10 parental antibody, at inhibiting tumor-derived GDF15-induced body weight reductions from multiple types of mouse tumor models (both syngeneic and patient-derived xenograft [PDX]). For the syngeneic models (KLN2015, B16b, or Renca), groups of male mice (DBA/2, BL/6 and BALB/c, respectively) were used, with n=10 per dose group (hIgG1 control or hz3P10 at 3 mg/kg). For the PDX models (GA0037 gastric, OV0276 ovarian and LI1098 liver), female BALB/c nude mice were used, a range of 9 to 20 per dose group (mIgG1 control or 3P10 at 3 mg/kg). Dose administration was Study Day 0 and 7 for gastric PDX, on Study Days 0, 7, 22, 28, 35, 49, and 63 for ovarian, every 2 weeks for 2 months for liver PDX, and weekly for up to 1 month for the syngeneic tumor models. In all models, GDF15 was increased by the tumors (FIGS. 9A and 9B) and body weight decreases were ameliorated by treatment with cither 3P10 (FIGS. 10A-C) or hz3P10 (FIGS. 11A-C).

### Example 5: Effect of 3P10 on Cisplatin-Induced Cachexia in Mice

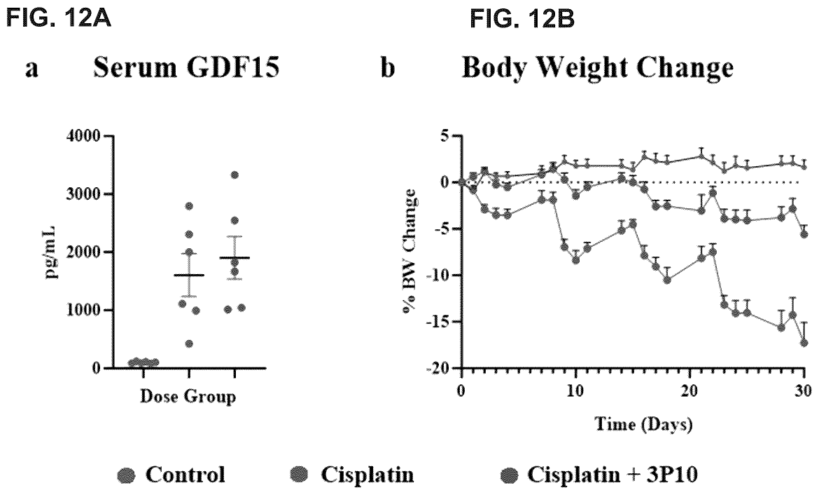

This study assessed the effect of 3P10 on the development of cisplatin-induced weight loss when administered weekly SC for 4 weeks in cisplatin treated mice. C57BL/6J mice (male, n=10/dose group, ˜17 weeks old at study start) were divided into groups of non-treated controls, cisplatin plus control antibody or cisplatin plus 3P10 treated mice. The control antibody and 3P10 were administered at 1 mg/kg SC weekly for 4 weeks while the cisplatin was administered 1 day after each dose at 5 mg/kg IP. Body weights, food consumption and serum GDF15 levels were measured throughout the study. After 1 month on study, serum GDF15 levels were 100 (+16), 1606 (+903) and 1905 (+899) pg/mL in the untreated controls, cisplatin plus control antibody and cisplatin plus 3P10 dose groups, respectively. By the end of the study, the cisplatin plus control antibody group had lost ˜20% of their initial body weight, while the cisplatin plus 3P10 group had lost ˜5% of their initial body weight, respectively. Untreated controls gained ˜2%. Food intake was similarly impacted, with the 3P10 group showing increased food consumption compared to the control antibody (FIGS. 12A-C).

### Example 6: Identification of No-Effect and Saturable Doses of Hz3P10 in Inhibition of GDF15-Induced Weight Loss in Rats

In this study, a human GDF15-Fc fusion protein was used to induce weight loss in rats, which were then administered hz3P10 to assess efficacy at ameliorating the weight loss. Male Sprague Dawley rats (n=6/dose group, ˜6 weeks old at study start) were administered cither placebo (PBS) or a GDF15-Fc fusion protein at a dose that reduces food intake and leads to weight loss in rats. Two days later, hz3P10 was administered as a single SC dose at 0 (PBS), 0.3, 1, 3 or 10 mg/kg.

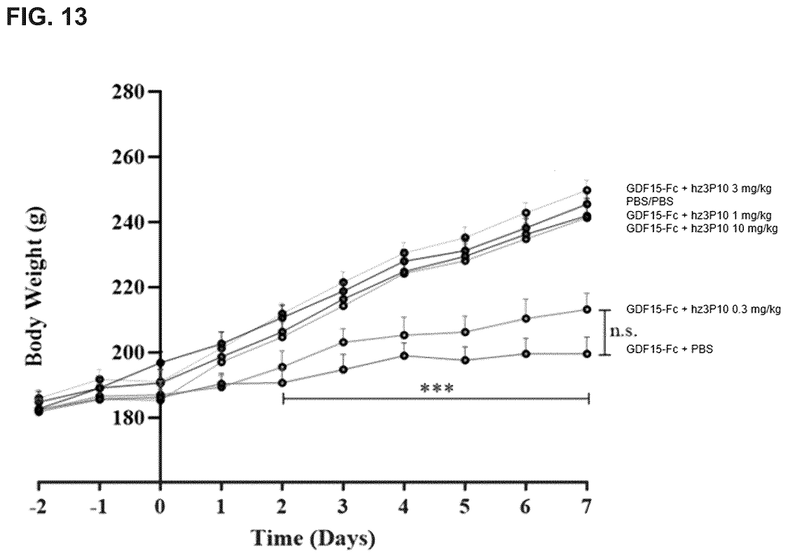

Animals administered the GDF15-Fc fusion protein and PBS showed significantly decreased weight gain compared to controls over the following week. Similarly, groups receiving GDF15-Fc fusion protein followed by 0.3 mg/kg hz3P10 showed a decrease in weight gain. However, there was no difference in weight gain among groups receiving placebo (PBS/PBS) or GDF15-Fc fusion protein followed by 1, 3 or 10 mg/kg hz3P10; all gained weight at a similar rate over the following week. At the end of 1 week, the group mean body weights were significantly lower for groups receiving GDF15-Fc fusion protein alone or with 0.3 mg/kg hz3P10, while the placebo control group was indistinguishable from the groups receiving GDF15-Fc fusion protein followed by 1, 3 or 10 mg/kg hz3P10 (FIG. 13). Food consumption was similarly affected, with NGM120 doses of 1, 3 or 10 mg/kg preventing the reductions in intake observed with GDF15-FC fusion protein.

### Example 7: Identification of No-Effect and Maximally Efficacious Doses of Hz3P10 in Inhibition of Cisplatin-Induced Weight Loss in Rats

To assess the efficacy of weekly subcutaneous doses of hz3P10 in blocking the weight loss effect of cisplatin on food intake and body weight in lean rats, Sprague Dawley rats (male, n=8/dose group, ˜6 weeks old at study start) were administered either 0 (PBS), 0.3, 1, 3 or 10 mg/kg hz3P10, followed 1 day later by PBS or cisplatin at 4 mg/kg. This continued weekly through 4 doses per group. Food intake and body weights were measured to assess the impact of cisplatin and the efficacy of hz3P10 at ameliorating the impact. In addition, serum GDF15 was measured 24 hours post each cisplatin dose.

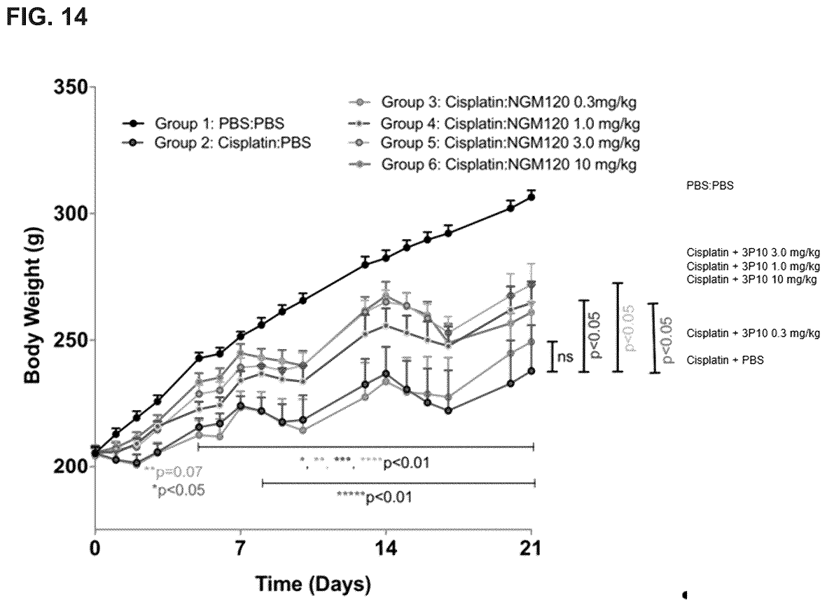

While all animals receiving cisplatin gained less weight than the placebo controls, hz3P10 at doses of 1, 3 and 10 mg/kg resulted in significantly increased body weight compared to the cisplatin: PBS group (FIG. 14). The 0.3 mg/kg hz3P10 group was not significantly different from the cisplatin: PBS group. Serum GDF15 levels were significantly increased compared to placebo controls in all animals that received cisplatin.

### Example 8: Predicted Human Efficacious Dose

The preclinical pharmacology studies described in the foregoing Examples in rodents were used for predicting a human efficacious dose. The minimal efficacious dose level in these studies was consistent between method of body weight induction and between species and was determined to be 1 mg/kg. Higher doses did not provide additional benefit. A dose of 0.3 mg/kg was consistently not efficacious. Based on the assumption that the body weight loss in rodent models is driven by the same mechanism as nausea and vomiting in humans, 1 mg/kg minimal effective exposure was used to guide human dose selection in humans.

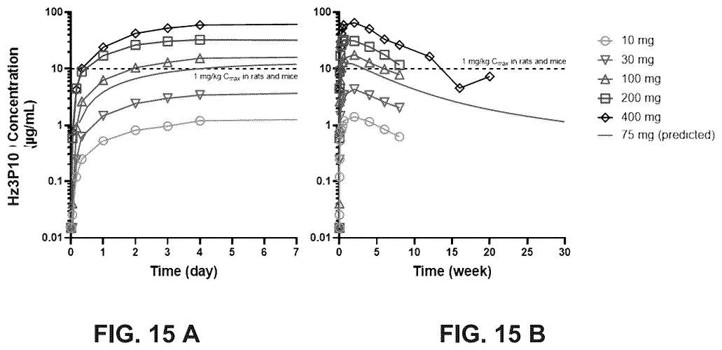

Mean serum Cₘₐₓ for a 1 mg/kg dose of hz3P10 was comparable between mice, (9.11 μg/mL) and rats (10.8 μg/mL) and the effective exposure of 9.96 μg/mL (averaging the mean Cₘₐₓ from those 2 species) was used as the target concentration threshold in the exposure margins calculations. Assuming an average of 75 kg body weight, a dose of 75 mg hz3P10 (equivalent to 1 mg/kg) is expected to achieve the effective exposure within 4 days (FIG. 15A) and maintain exposure within the efficacious range until ˜3 weeks from dosing (FIG. 15B). It is understood that the absolute dose may be increased or decreased based on the body weight of the subject in order to achieve a target dose of about 1 mg/kg.

### Example 9: Pharmacokinetics and Drug Metabolism In Humans

A single ascending dose (SAD) study was conducted in which a total of 48 healthy adult human volunteers received a single subcutaneous dose of 10 mg, 30 mg, 100 mg, 200 mg, or 400 mg of the humanized anti-GFRAL antibody 3P10 (comprising a heavy chain of SEQ ID NO:2010, and a light chain of SEQ ID NO:2012). A multiple ascending dose (MAD) study was conducted in which a total of 44 healthy adult human volunteers were administered by subcutaneous injection 10 mg, 30 mg, 100 mg, or 200 mg, once every four weeks (Q4W) for a total of three doses.

In the SAD portion of the study, the median Tₘₐₓ ranged from 4 to 15 days following a single SC dose of hz3P10. Mean Cₘₐₓ and AUC_{0-56d} values increased in an approximately dose-proportional manner between the 10 mg, 30 mg, 100 mg, 200 mg, and 400 mg dose levels. Across the dose groups, the mean apparent oral clearance (CL/F) had a range of 87.9 to 118 mL/day and the t_{1/2} (geometric mean) ranged from 28.3-40.0 days.

In the MAD portion of the study, the median Tₘₐₓ was approximately 7 days for the first and 3rd dose (i.e., Days 1 and 57, respectively). Exposure (based on Cₘₐₓ and AUC_{0-28d}) after the 3rd dose following Q4W dosing of 10 mg, 30 mg, 100 mg, or 200 mg hz3P10 was approximately 2-fold higher than that after the first dose. Mean accumulation after the 4^{rd} dose ranged from 1.6 to 2.0 for Cₘₐₓ values, and 1.7 to 2.2 for AUC_{0-28d} values.

All publications, patents, patent applications, internet sites, and accession numbers/database sequences including both polynucleotide and polypeptide sequences cited herein are hereby incorporated by reference in their entirety for all purposes to the same extent as if each individual publication, patent, patent application, internet site, or accession number/database sequence were specifically and individually indicated to be so incorporated by reference.

1. A method for treating hyperemesis gravidarum in a human subject in need thereof, the method comprising administering to the human subject a therapeutically effective amount of an antibody that specifically binds human GDNF Family Receptor Alpha-Like (GFRAL), wherein the antibody comprises a heavy chain variable region (VH) and a light chain variable region (VL), and wherein the VH comprises a VH complementarity determining region (CDR) 1, a VH CDR2, and a VH CDR3 from the amino acid sequence set forth in SEQ ID NO:1982, and the VL comprises a VL CDR1, a VL CDR2, and a VL CDR3 from the amino acid sequence set forth in SEQ ID NO:1997.

2. A method for treating or reducing nausea and/or vomiting in a human subject in need thereof, the method comprising administering to the human subject a therapeutically effective amount of an antibody that specifically binds human GFRAL, wherein the antibody comprises a heavy chain variable region (VH) and a light chain variable region (VL), and wherein the VH comprises a VH complementarity determining region (CDR) 1, a VH CDR2, and a VH CDR3 from the amino acid sequence set forth in SEQ ID NO:1982, and the VL comprises a VL CDR1, a VL CDR2, and a VL CDR3 from the amino acid sequence set forth in SEQ ID NO:1997.

3. The method of claim 2, wherein the nausea and/or vomiting is nausea and vomiting of pregnancy (NVP).

4. The method of claim 3, wherein the wherein the NVP is mild NVP, moderate NVP, or severe NVP.

5. The method of claim 1, wherein the administering is performed subcutaneously or intravenously.

6. (canceled)

7. The method of claim 1, wherein the therapeutically effective amount of the antibody is about 100 mg, about 75 mg, or about 30 mg.

8. (canceled)

9. The method of claim 1, wherein the therapeutically effective amount of the antibody is 75 mg.

10. The method of claim 1, wherein the method comprises administering only a single dose of the therapeutically effective amount of the antibody to the human subject.

11. The method of claim 1, wherein the method comprises administering more than one dose of the therapeutically effective amount of the antibody to the human subject, optionally and wherein each dose is administered once every about four weeks.

12. The method of claim 1, wherein a biological sample obtained from the human subject has an elevated level of GDF15 compared to a control level of GDF15 in a control biological sample obtained from one or more humans not suffering from nausea and vomiting of pregnancy, hyperemesis gravidarum, nausea, or vomiting, optionally wherein the biological sample is serum, plasma, or blood.

13. (canceled)

14. (canceled)

15. (canceled)

16. (canceled)

17. (canceled)

18. (canceled)

19. (canceled)

20. The method of claim 1, wherein:
(a) the VH CDR1, VH CDR2, VH CDR3, VL CDR1, VL CDR2, and VL CDR3 comprise the amino acid sequences set forth in SEQ ID NO:46, SEQ ID NO:137, SEQ ID NO:225, SEQ ID NO:301, SEQ ID NO:376, and SEQ ID NO:426, respectively;
(b) the VH CDR1, VH CDR2, VH CDR3, VL CDR1, VL CDR2, and VL CDR3 comprise the amino acid sequences set forth in SEQ ID NO:47, SEQ ID NO:138, SEQ ID NO:226, SEQ ID NO:302, SEQ ID NO:377, and SEQ ID NO:426, respectively;
(c) the VH CDR1, VH CDR2, VH CDR3, VL CDR1, VL CDR2, and VL CDR3 comprise the amino acid sequences set forth in SEQ ID NO:48, SEQ ID NO:137, SEQ ID NO:225, SEQ ID NO:301, SEQ ID NO:376, and SEQ ID NO:426, respectively;
(d) the VH CDR1, VH CDR2, VH CDR3, VL CDR1, VL CDR2, and VL CDR3 comprise the amino acid sequences set forth in SEQ ID NO:49, SEQ ID NO:139, SEQ ID NO:227, SEQ ID NO:303, SEQ ID NO:377, and SEQ ID NO:427, respectively;
(e) the VH CDR1, VH CDR2, VH CDR3, VL CDR1, VL CDR2, and VL CDR3 comprise the amino acid sequences set forth in SEQ ID NO:50, SEQ ID NO:140, SEQ ID NO:228, SEQ ID NO:304, SEQ ID NO:378, and SEQ ID NO:428, respectively; or
(f) the VH CDR1, VH CDR2, VH CDR3, VL CDR1, VL CDR2, and VL CDR3 comprise the amino acid sequences set forth in SEQ ID NO:46, SEQ ID NO:141, SEQ ID NO:225, SEQ ID NO:301, SEQ ID NO:376, and SEQ ID NO:426, respectively.

21. The method of claim 1, wherein:
the VH comprises an amino acid sequence having at least 80% sequence identity to the amino acid sequence set forth in any one of SEQ ID NOs:1978-1988 and the VL comprises an amino acid sequence having at least 80% sequence identity to the amino acid sequence set forth in any one of SEQ ID NOs:1990-2000.

22. The method of claim 1, wherein the VH comprises the amino acid sequence set forth in SEQ ID NO:1982 and the VL comprises the amino acid sequence set forth in SEQ ID NO:1997.

23. A method for treating hyperemesis gravidarum or for treating or reducing nausea and/or vomiting in a human subject in need thereof, the method comprising administering to the human subject a therapeutically effective amount of an antibody that specifically binds human GDNF Family Receptor Alpha-Like (GFRAL) wherein the antibody comprises a heavy chain variable region (VH) and a light chain variable region (VL), and wherein:
(a) the VH comprises a VH complementarity determining region (CDR) 1, a VH CDR2, and a VH CDR3 from the amino acid sequence set forth in any one of SEQ ID NOs:7 and 1957-1965, and the VL comprises a VL CDR1, a VL CDR2, and a VL CDR3 from the amino acid sequence set forth in any one of SEQ ID NOs:8 and 1967-1976;
(b) the VH comprises a VH CDR1, a VH CDR2, and a VH CDR3 from the amino acid sequence set forth in SEQ ID NO:21, and the VL comprises a VL CDR1, a VL CDR2, and a VL CDR3 from the amino acid sequence set forth in SEQ ID NO:22;
(c) the VH comprises a VH CDR1, a VH CDR2, and a VH CDR3 from the amino acid sequence set forth in SEQ ID NO:23, and the VL comprises a VL CDR1, a VL CDR2, and a VL CDR3 from the amino acid sequence set forth in SEQ ID NO:24;
(d) the VH comprises a VH CDR1, a VH CDR2, and a VH CDR3 from the amino acid sequence set forth in SEQ ID NO:25, and the VL comprises a VL CDR1, a VL CDR2, and a VL CDR3 from the amino acid sequence set forth in SEQ ID NO:26;
(e) the VH comprises a VH CDR1, a VH CDR2, and a VH CDR3 from the amino acid sequence set forth in SEQ ID NO:37, and the VL comprises a VL CDR1, a VL CDR2, and a VL CDR3 from the amino acid sequence set forth in SEQ ID NO:38;
(f) the VH comprises a VH CDR1, a VH CDR2, and a VH CDR3 from the amino acid sequence set forth in SEQ ID NO:39, and the VL comprises a VL CDR1, a VL CDR2, and a VL CDR3 from the amino acid sequence set forth in SEQ ID NO:40;
(g) the VH comprises a VH CDR1, a VH CDR2, and a VH CDR3 from the amino acid sequence set forth in any one of SEQ ID NOs:15 and 1936-1941, and the VL comprises a VL CDR1, a VL CDR2, and a VL CDR3 from the amino acid sequence set forth in any one of SEQ ID NOs:16 and 1943-1947;
(h) the VH comprises a VH CDR1, a VH CDR2, and a VH CDR3 from the amino acid sequence set forth in any one of SEQ ID NOs:1 and 1918-1927, and the VL comprises a VL CDR1, a VL CDR2, and a VL CDR3 from the amino acid sequence set forth in any one of SEQ ID NOs:2 and 1929-1934; or
(i) the VH comprises a VH CDR1, a VH CDR2, and a VH CDR3 from the amino acid sequence set forth in SEQ ID NO:11, and the VL comprises a VL CDR1, a VL CDR2, and a VL CDR3 from the amino acid sequence set forth in SEQ ID NO:12;
(j) the VH comprises a VH CDR1, a VH CDR2, and a VH CDR3 from the amino acid sequence set forth in SEQ ID NO:5, and the VL comprises a VL CDR1, a VL CDR2, and a VL CDR3 from the amino acid sequence set forth in SEQ ID NO:6;
(k) the VH comprises a VH CDR1, a VH CDR2, and a VH CDR3 from the amino acid sequence set forth in SEQ ID NO:9, and the VL comprises a VL CDR1, a VL CDR2, and a VL CDR3 from the amino acid sequence set forth in SEQ ID NO:10;
(l) the VH comprises a VH CDR1, a VH CDR2, and a VH CDR3 from the amino acid sequence set forth in SEQ ID NO:13, and the VL comprises a VL CDR1, a VL CDR2, and a VL CDR3 from the amino acid sequence set forth in SEQ ID NO:14;
(m) the VH comprises a VH CDR1, a VH CDR2, and a VH CDR3 from the amino acid sequence set forth in SEQ ID NO:17, and the VL comprises a VL CDR1, a VL CDR2, and a VL CDR3 from the amino acid sequence set forth in SEQ ID NO:18;
(n) the VH comprises a VH CDR1, a VH CDR2, and a VH CDR3 from the amino acid sequence set forth in SEQ ID NO:19, and the VL comprises a VL CDR1, a VL CDR2, and a VL CDR3 from the amino acid sequence set forth in SEQ ID NO:20;
(o) the VH comprises a VH CDR1, a VH CDR2, and a VH CDR3 from the amino acid sequence set forth in SEQ ID NO:27, and the VL comprises a VL CDR1, a VL CDR2, and a VL CDR3 from the amino acid sequence set forth in SEQ ID NO:28;
(p) the VH comprises a VH CDR1, a VH CDR2, and a VH CDR3 from the amino acid sequence set forth in SEQ ID NO:29, and the VL comprises a VL CDR1, a VL CDR2, and a VL CDR3 from the amino acid sequence set forth in SEQ ID NO:30;
(g) the VH comprises a VH CDR1, a VH CDR2, and a VH CDR3 from the amino acid sequence set forth in SEQ ID NO:31, and the VL comprises a VL CDR1, a VL CDR2, and a VL CDR3 from the amino acid sequence set forth in SEQ ID NO:32;
(r) the VH comprises a VH CDR1, a VH CDR2, and a VH CDR3 from the amino acid sequence set forth in SEQ ID NO:33, and the VL comprises a VL CDR1, a VL CDR2, and a VL CDR3 from the amino acid sequence set forth in SEQ ID NO:34;
(s) the VH comprises a VH CDR1, a VH CDR2, and a VH CDR3 from the amino acid sequence set forth in SEQ ID NO:35, and the VL comprises a VL CDR1, a VL CDR2, and a VL CDR3 from the amino acid sequence set forth in SEQ ID NO:36;
(t) the VH comprises a VH CDR1, a VH CDR2, and a VH CDR3 from the amino acid sequence set forth in SEQ ID NO:480, and the VL comprises a VL CDR1, a VL CDR2, and a VL CDR3 from the amino acid sequence set forth in SEQ ID NO:481;
(u) the VH comprises a VH CDR1, a VH CDR2, and a VH CDR3 from the amino acid sequence set forth in SEQ ID NO:482, and the VL comprises a VL CDR1, a VL CDR2, and a VL CDR3 from the amino acid sequence set forth in SEQ ID NO:483;
(v) the VH comprises a VH CDR1, a VH CDR2, and a VH CDR3 from the amino acid sequence set forth in SEQ ID NO:484, and the VL comprises a VL CDR1, a VL CDR2, and a VL CDR3 from the amino acid sequence set forth in SEQ ID NO:485; or
(w) the VH comprises a VH CDR1, a VH CDR2, and a VH CDR3 from the amino acid sequence set forth in SEQ ID NO:486, and the VL comprises a VL CDR1, a VL CDR2, and a VL CDR3 from the amino acid sequence set forth in SEQ ID NO:487.

24. (canceled)

25. (canceled)

26. (canceled)

27. (canceled)

28. (canceled)

29. (canceled)

30. (canceled)

31. The method of claim 1, wherein the antibody comprises two heavy chains and two light chains.

32. The method of claim 1, wherein the antibody is a human IgG1, human IgG2, or human IgG4 antibody.

33. The method of claim 1, wherein the antibody is a human IgG1 antibody and comprises a human kappa light chain constant region or a human lambda light chain constant region.

34. (canceled)

35. (canceled)

36. The method of claim 1, wherein the antibody is a Fab, Fab′, F(ab′)₂, Fv, scFv, (scFv)₂, single chain antibody, dual variable region antibody, single variable region antibody, linear antibody, diabody, or a V region antibody.

37. The method of claim 1, wherein the antibody comprises a heavy chain comprising an amino acid sequence having at least 80% sequence identity to the amino acid sequence set forth in SEQ ID NO:2010 and a light chain comprising an amino acid sequence having at least 80% sequence identity to the amino acid sequence set forth in SEQ ID NO:2012.

38. The method of claim 1, wherein the antibody comprises a heavy chain comprising the amino acid sequence set forth in SEQ ID NO: 2010 and a light chain comprising the amino acid sequence set forth in SEQ ID NO: 2012.

39. The method of claim 1, wherein the antibody comprises a heavy chain consisting of the amino acid sequence set forth in SEQ ID NO: 2010 and a light chain consisting of the amino acid sequence set forth in SEQ ID NO: 2012.

40. (canceled)

41. The method of claim 1, wherein the human subject (a) is unresponsive to prior administration of an antiemetic or (b) has unresolved nausea, vomiting, or a combination thereof after prior administration of an antiemetic.

42. The method of claim 1, further comprising administering one or more of: doxylamine, pyridoxine, a dual release formulation of doxylamine and pyridoxine, ondansetron, a dopamine antagonist, diphenhydramine, droperidol, methylprednisolone, mirtazapine, dimenhydrinate, chlorpromazine, prochlorperazine, a steroid therapy, an anti-emetic, vitamin B1, vitamin B6, folic acid, vitamin K, vitamin D, Magnesium, and ginger.

43. A composition comprising a combination of an anti-GFRAL antibody and one or more of: doxylamine, pyridoxine, a dual release formulation of doxylamine and pyridoxine, ondansetron, a dopamine antagonist, diphenhydramine, droperidol, methylprednisolone, mirtazapine, dimenhydrinate, chlorpromazine, prochlorperazine, a steroid therapy, an anti-emetic, vitamin B1, vitamin B6, folic acid, vitamin K, vitamin D, Magnesium, and ginger, optionally wherein the anti-GFRAL antibody comprises a VH and a VL, wherein the VH comprises the amino acid sequence set forth in SEQ ID NO:1982 and the VL comprises the amino acid sequence set forth in SEQ ID NO:1997.


SEQUENCE LISTING (2012 sequences)

SEQ ID NO: 1 - protein, 121 aa, synthetic construct
QMQLKQSGPGLVQPSQSLSITCTVSGFSLNDYGVHWIRQSPGKGLEWLGVIWSGGRTDYNAAFISRLSISKDNSKSQVFFKMSSLQPQDTAIYYCARWALYFLYGGSMDYWGQGTSVTVSS

SEQ ID NO: 2 - protein, 113 aa, synthetic construct
DVVLTQTPLSLPVSPGDQASISCRSSQSLVHSSGITYLHWYLQKPGQSPKLLIYKLSNRFSGVPDRFSGSGSGTDFTLKISRVEAEDLGVYFCSQSTHVPPWTFGGGTKLEIK

SEQ ID NO: 3 - protein, 118 aa, synthetic construct
QIQLVQSGPELKKPGETVKISCKASGYTFTDYGVIWVKQAPGKALKWMGWINTYTGEPTYADDLKGRFAFSLETSASSASLQINNLKNEDTATYFCARRYGPEDIDYWGQGTTLTVSS

SEQ ID NO: 4 - protein, 111 aa, synthetic construct
DIVLTQSPVSLAVSLGQRATISCRASESVDNYGISFMSWFQQKPGQPPKLLIYAASHQGSGVPARFSGSGSGTDFSLNIHPMEEDDSAMYFCLQSKEVPWTFGGGTKLEIK

SEQ ID NO: 5 - protein, 114 aa, synthetic construct
QIQLVQSGPELKKPGETVKISCKASGYPFTIYGMNWVEQAPGKGLKWMGWINTYSGVPTYADDFKGRFAFSLETSASTAYLQINNLKDEDTATYFCASATGNYWGQGTTLTVSS

SEQ ID NO: 6 - protein, 107 aa, synthetic construct
DIQMTQSPSSLSASLGERVSLTCRASQDIGSSLNWLQQEPDGTIKRLIYATSSLDSGVPKRFSGSRSGSDYSLTISSLESEDFVDYYCLQYASSPYTFGGGTKVEIK

SEQ ID NO: 7 - protein, 122 aa, synthetic construct
QVQLKQSGTELVRPGASVKLSCKASGYTFTDYYINWVKQRPGQGLEWIARIYPGNGNTYHNEKFKGKATLTAEKSSSTAYMQLSSLTSEDSAVYFCAREGLYYDYDRYFDYWGQGTALTVSS

SEQ ID NO: 8 - protein, 111 aa, synthetic construct
NIVLTQSPASLAVSLGQRATISCRASESVDTYGNSFMHWYQQKPGQPPKLLIYLASNLESGVPARFSGSGSRTDFTLTIDPVEADDAATYYCHQNNEDPPAFGGGTKLEIK

SEQ ID NO: 9 - protein, 118 aa, synthetic construct
QVQLQQSGPELMKPGASVILSCKAIGYTFTDYWIEWVKERPGHGLEWIGEILLGSDSIHFNEKFKGKATISADTSSNTAYMQLSSLTTEDSAIYYCVRQDWNWYFDVWGTGTTVTVSS

SEQ ID NO: 10 - protein, 112 aa, synthetic construct
DVVMTQTPLTLSVTIGHPASISCKSSQSLLDFDGKTYLNWLFQRPGQSPKRLFYLVSKLDSGVPDRFTGSGSGTDFTLKISRVEAEDLGVYYCWQGTHFPRTFGGGTKLEIK

SEQ ID NO: 11 - protein, 116 aa, synthetic construct
QVQLKESGPGLVAPSQSLSITCTVSGFSLSRYSVHWVRQPPGKGLEWLGMIWGFGSTDYNSALKSRLSITKDNSKSQFFLKMNSLQTDDTAMYYCARIHTTAGSYWGQGTLVTVSA

SEQ ID NO: 12 - protein, 107 aa, synthetic construct
DIVMTQSQKFMSTSIGDRVSVTCKASQNVGTNVAWYQQKPGQSPKALVYSTSYRYSGVPDRFTGSGSGTDFTLTISNVQSEDLAEYFCHQYNSYPLTFGAGTKLELK

SEQ ID NO: 13 - protein, 118 aa, synthetic construct
QVQLQQSGAELAKPGASVKMSCKTSGYTFTDYWIHWVKQRPGQGLEWIGYINPNSNYAEYNQKFKVKATLTADKSSSTAYLQLSRLTSEDSAVYYCARFDWNWYFHVWGAGSTVTVSS

SEQ ID NO: 14 - protein, 112 aa, synthetic construct
DVALTQIPLTLSVTVGQPASISCKSSQSLSDSDGKTYLNWLLQKPGQSPKRLIYLVSRLGSGVPDRFTGSGSGADFTLKISRVEAEDLGVYYCWQGTHFPQTFGGGTKLEIK

SEQ ID NO: 15 - protein, 125 aa, synthetic construct
QVQLQQSGPDLVKPGASVKISCKASGYTFTSYWVNWMKQRPGKGLEWIGRIYPGDGDTNYNGKFKGKATLTADKSSSTAYMQLSSLTSEDSAVYFCARAYLLRLRRTGYYAMDYWGQGTSVTVSS

SEQ ID NO: 16 - protein, 112 aa, synthetic construct
DVVLTQTPLSLPVNIGDQASISCKSTKSLLNSDEFTYLDWYLQKPGQSPQLLIFLVSNRFSGVPDRFSGSGSGTDFTLKISRVEAEDLGVYYCFQSNYLPYTFGGGTKLEIK

SEQ ID NO: 17 - protein, 121 aa, synthetic construct
QIQLVQSGPELKKPGETVKISCKASGYTFTTYGMSWVKQAPGKIFKWMGWINTYSGVPTFVDDFRGRFAFSLETSASTAYLQIGNLKNEDTATYFCARRSSYYPYWYFDVWGTGTTVTVSS

SEQ ID NO: 18 - protein, 107 aa, synthetic construct
DIQMTQSPASLSASVGETVTITCRPSENIYSYLTWFQQEQGKSPQLLVYNAQTLAEGVPSRFSGSGSGTHFSLKINSLQPEDFGTYYCQHYYGYPFTFGSGTKLEIK

SEQ ID NO: 19 - protein, 114 aa, synthetic construct
QNQLVQSGPELKKPGEIVKISCKTSGYTFTDYSMHWVKKTPGKGFKWMGWINTETGEPTYADDFKGRFAFSLETSANTAHLQITNLKNEDTATYFCVKGTLNYWGQGTTLTVSS

SEQ ID NO: 20 - protein, 107 aa, synthetic construct
DIKMTQSPSSMYASLGERVTITCKASQDIKSYLNWFQQKPGKSPKTLIYRTKRLVDGVPSRFSGSGSGQDYSLTVSSLEYDDVGIYYCLQYVEFPLTFGDGTKLELK

SEQ ID NO: 21 - protein, 120 aa, synthetic construct
QAQLQQSGAELVKPGASVKLSCKASGYSFTSYNIDWVRQRPEQGLEWIGWIFPGDGSTKYNEKFKGQATLTTDKSSSTTYIHLSRLTSEDSAVYFCARSGIYYGSHFVYWGQGTLVTVSA

SEQ ID NO: 22 - protein, 112 aa, synthetic construct
DVVMTQTPLTLSVTIGQSASISCRSSQSLLDSDGKTYLNWLLQRPGQSPKRLIYLVSKVDSGVPDRFTGSGSGTDFTLKISRVEAEDLGVYFCWQGTHFPLTFGAGTKLELK

SEQ ID NO: 23 - protein, 120 aa, synthetic construct
QVQLQQSGSELVKPGTSMKLSCKASGYTFTSYNINWVRLRPEQGLEWIGWIFPGDDSIKYNENFRGKATLTTDKSSSTAYMHLSRLTSDDSAVYFCARSGIFYGNNFAYWGQGTLVTVSA

SEQ ID NO: 24 - protein, 112 aa, synthetic construct
DVVMTQAPLILSVTIGQPASISCKSSQSLLDGDGETYLSWLLQRPGQSPKRLIYLVSKLDSGVPDRFTGSGSGTDFTLKISRVEAEDLGVYYCCQSTHFPLTFGAGTKLELK

SEQ ID NO: 25 - protein, 119 aa, synthetic construct
QVHLQQPGAELVKPGASVKLSCKASGFTFTGYNINWVRLRPEQGLEWIGWIFPGDDNAKYNEKFKGKATLTTDKSSNTAYMQLSRLTSEDSAVYFCARTPVLSNYFDYWGQGTTLTVSS

SEQ ID NO: 26 - protein, 106 aa, synthetic construct
DIQMTQSPSSLSASLGGKVTITCKASQDISKYISWYQHKPGKSPRLLIHYTSTLQPGIPSRFSGSGSGRDYSFSISNLEPEDIATYYCLQYDNLYTFGGGTKLEIK

SEQ ID NO: 27 - protein, 125 aa, synthetic construct
QVQLQQSGPDLVKPGASVKISCKASGYAFTSYWMNWVKQRPGKGLEWIGRIYPGDGDTNYNGKFKGKATLTADKSSSTAYMQLSSLTSEDSAVYFCARAYLLRLRRTGYYAMDYWGQGTSVTVSS

SEQ ID NO: 28 - protein, 112 aa, synthetic construct
DVVLTQTPLSLPVNIGDQASISCKSTKSLLNSDEFTYLDWYLQKPGQSPQLLIYLVSNRFSGVPDRFSGSGSGTDFTLKISRVEAEDLGVYYCFQSNYLPYTFGGGTKLEIK

SEQ ID NO: 29 - protein, 118 aa, synthetic construct
EVKLVESGGGLVQPGGSLKLSCAASGFTFSDYFMFWVRQTPEKRLEWVAYISNDGDSTYYPDTVQGRFTISRDNAKNTLYLQMSRLRSEDTAMYYCTRQGAQATLDYWGQGTTLTVSS

SEQ ID NO: 30 - protein, 103 aa, synthetic construct
QIVLTQSPAIMSASLGEEITLTCSASSSVFYMHWYQQKSGTSPKLLIYSTSNLASGIPSRFSGSGSGTFYSLTISSVEAEDAADYYCHQWSSTFGGGTKLEIK

SEQ ID NO: 31 - protein, 122 aa, synthetic construct
QVQLQQSGVELARPGAAVKLSCKASGYTFANYGLTWVKQRTGQGLEWIGEIYPGSGHTHYNEDFKGKATLTADRSSSTAYMELRSLTSEDSAVYFCARRIQLLLPVGGFVYWGQGTLVTVSA

SEQ ID NO: 32 - protein, 107 aa, synthetic construct
DFVLTQSPATLSVTPGDSVSLSCRASQSISNNLHWYQQKSHESPRLLIKYASQSISGIPSRFSGSGSGTDFTLSINSVETEDFGVYFCQQSNSWPHTFGGGTKLEIK

SEQ ID NO: 33 - protein, 119 aa, synthetic construct
EVKLVESGGGLVKPGGSLKLSCAASGFTFSTYAMSWVRQTPEKRLEWVASITSGGTTYYTDSVKGRFTISRDNARNILYLQMSSLRSEDTAMYYCARDGNFYYYGMDYWGQGTSVTVSS

SEQ ID NO: 34 - protein, 106 aa, synthetic construct
DIVMTQSQKFMSTSVGDRVSITCKASQNVGTAVAWYQQKPGQSPKILIYSASNRFTGVPDRFTGSGSGTDFTLTISNMQSEDLADYFCQQYSSYFTFGGGTKLELK

SEQ ID NO: 35 - protein, 120 aa, synthetic construct
QVQLQQSGAELLKPGASVKLSCKASGYTFTTYWMHWVKQRPGQGLEWIGMIHPNSGSSNYNEKFKNKATLTVDKSSSTAYMQLSSLTSEDSAVYFCARSDYGFIPYFDYWGQGTTLTVSS

SEQ ID NO: 36 - protein, 108 aa, synthetic construct
DILLTQSPAILSVSPGERVSFSCRASQSIGTSIHWYQQRTNGSPRLLIKYASESISGIPSRFSGSGSGTDFTLIINSVESEDIADYYCQQSNSWPTFTFGAGTKLELK

SEQ ID NO: 37 - protein, 120 aa, synthetic construct
QVQLHQPGAELVKPGASVKLSCKTSGYTFTSYWMQWVKQRPGQGLEWIGEIDPSDSYTNYNQKFKGKATLTVDTSSTTAYMQLSSLTSEDSAVYYCARPLDRSAYYFDYWGQGTTLTVSS

SEQ ID NO: 38 - protein, 111 aa, synthetic construct
DIVLTQSPASLAVSLGQRATISCRASESVDFSGNSFMHWYQQKPGQPPKLLIYRASNLDSGIPARFSGVGSRTDFTLTINPVEADDVATYYCQQSNEDPYTFGGGTKLEIE

SEQ ID NO: 39 - protein, 119 aa, synthetic construct
DVQLQESGPGLVKPSQSLSLTCSVTGYSITSGYYWNWIRQFPGNKLEWMGHIANDGSNYYNPFLKHRVSITRDTSKNQFFLKLNSVTIQDTATYYCARGGSYFDYVDYWGQGTTLTVSS

SEQ ID NO: 40 - protein, 107 aa, synthetic construct
DIQMTQTTSSLSASLGDRVTINCRASQDISNYLNWYQQKPDGTVKLLIYYTSRLHSGVPSRFSGSGSGTDYSLTITNLEQEDIATYFCQQGNTLPFTFGSGTKLEIK

SEQ ID NO: 41 - protein, 10 aa, synthetic construct
GFSLNDYGVH

SEQ ID NO: 42 - protein, 8 aa, synthetic construct
GFSLNDYG

SEQ ID NO: 43 - protein, 5 aa, synthetic construct
DYGVH

SEQ ID NO: 44 - protein, 7 aa, synthetic construct
GFSLNDY

SEQ ID NO: 45 - protein, 6 aa, synthetic construct
NDYGVH

SEQ ID NO: 46 - protein, 10 aa, synthetic construct
GYTFTDYGVI

SEQ ID NO: 47 - protein, 8 aa, synthetic construct
GYTFTDYG

SEQ ID NO: 48 - protein, 5 aa, synthetic construct
DYGVI

SEQ ID NO: 49 - protein, 7 aa, synthetic construct
GYTFTDY

SEQ ID NO: 50 - protein, 6 aa, synthetic construct
TDYGVI

SEQ ID NO: 51 - protein, 10 aa, synthetic construct
GYPFTIYGMN

SEQ ID NO: 52 - protein, 8 aa, synthetic construct
GYPFTIYG

SEQ ID NO: 53 - protein, 5 aa, synthetic construct
IYGMN

SEQ ID NO: 54 - protein, 7 aa, synthetic construct
GYPFTIY

SEQ ID NO: 55 - protein, 6 aa, synthetic construct
TIYGMN

SEQ ID NO: 56 - protein, 10 aa, synthetic construct
GYTFTDYYIN

SEQ ID NO: 57 - protein, 8 aa, synthetic construct
GYTFTDYY

SEQ ID NO: 58 - protein, 5 aa, synthetic construct
DYYIN

SEQ ID NO: 59 - protein, 6 aa, synthetic construct
TDYYIN

SEQ ID NO: 60 - protein, 10 aa, synthetic construct
GYTFTDYWIE

SEQ ID NO: 61 - protein, 8 aa, synthetic construct
GYTFTDYW

SEQ ID NO: 62 - protein, 5 aa, synthetic construct
DYWIE

SEQ ID NO: 63 - protein, 6 aa, synthetic construct
TDYWIE

SEQ ID NO: 64 - protein, 10 aa, synthetic construct
GFSLSRYSVH

SEQ ID NO: 65 - protein, 8 aa, synthetic construct
GFSLSRYS

SEQ ID NO: 66 - protein, 5 aa, synthetic construct
RYSVH

SEQ ID NO: 67 - protein, 7 aa, synthetic construct
GFSLSRY

SEQ ID NO: 68 - protein, 6 aa, synthetic construct
SRYSVH

SEQ ID NO: 69 - protein, 10 aa, synthetic construct
GYTFTDYWIH

SEQ ID NO: 70 - protein, 5 aa, synthetic construct
DYWIH

SEQ ID NO: 71 - protein, 6 aa, synthetic construct
TDYWIH

SEQ ID NO: 72 - protein, 10 aa, synthetic construct
GYTFTSYWVN

SEQ ID NO: 73 - protein, 8 aa, synthetic construct
GYTFTSYW

SEQ ID NO: 74 - protein, 5 aa, synthetic construct
SYWVN

SEQ ID NO: 75 - protein, 7 aa, synthetic construct
GYTFTSY

SEQ ID NO: 76 - protein, 6 aa, synthetic construct
TSYWVN

SEQ ID NO: 77 - protein, 10 aa, synthetic construct
GYTFTTYGMS

SEQ ID NO: 78 - protein, 8 aa, synthetic construct
GYTFTTYG

SEQ ID NO: 79 - protein, 5 aa, synthetic construct
TYGMS

SEQ ID NO: 80 - protein, 7 aa, synthetic construct
GYTFTTY

SEQ ID NO: 81 - protein, 6 aa, synthetic construct
TTYGMS

SEQ ID NO: 82 - protein, 10 aa, synthetic construct
GYTFTDYSMH

SEQ ID NO: 83 - protein, 8 aa, synthetic construct
GYTFTDYS

SEQ ID NO: 84 - protein, 5 aa, synthetic construct
DYSMH

SEQ ID NO: 85 - protein, 6 aa, synthetic construct
TDYSMH

SEQ ID NO: 86 - protein, 10 aa, synthetic construct
GYSFTSYNID

SEQ ID NO: 87 - protein, 8 aa, synthetic construct
GYSFTSYN

SEQ ID NO: 88 - protein, 5 aa, synthetic construct
SYNID

SEQ ID NO: 89 - protein, 7 aa, synthetic construct
GYSFTSY

SEQ ID NO: 90 - protein, 6 aa, synthetic construct
TSYNID

SEQ ID NO: 91 - protein, 10 aa, synthetic construct
GYTFTSYNIN

SEQ ID NO: 92 - protein, 8 aa, synthetic construct
GYTFTSYN

SEQ ID NO: 93 - protein, 5 aa, synthetic construct
SYNIN

SEQ ID NO: 94 - protein, 6 aa, synthetic construct
TSYNIN

SEQ ID NO: 95 - protein, 10 aa, synthetic construct
GFTFTGYNIN

SEQ ID NO: 96 - protein, 8 aa, synthetic construct
GFTFTGYN

SEQ ID NO: 97 - protein, 5 aa, synthetic construct
GYNIN

SEQ ID NO: 98 - protein, 7 aa, synthetic construct
GFTFTGY

SEQ ID NO: 99 - protein, 6 aa, synthetic construct
TGYNIN

SEQ ID NO: 100 - protein, 10 aa, synthetic construct
GYAFTSYWMN

SEQ ID NO: 101 - protein, 8 aa, synthetic construct
GYAFTSYW

SEQ ID NO: 102 - protein, 5 aa, synthetic construct
SYWMN

SEQ ID NO: 103 - protein, 7 aa, synthetic construct
GYAFTSY

SEQ ID NO: 104 - protein, 6 aa, synthetic construct
TSYWMN

SEQ ID NO: 105 - protein, 10 aa, synthetic construct
GFTFSDYFMF

SEQ ID NO: 106 - protein, 8 aa, synthetic construct
GFTFSDYF

SEQ ID NO: 107 - protein, 5 aa, synthetic construct
DYFMF

SEQ ID NO: 108 - protein, 7 aa, synthetic construct
GFTFSDY

SEQ ID NO: 109 - protein, 6 aa, synthetic construct
SDYFMF

SEQ ID NO: 110 - protein, 10 aa, synthetic construct
GYTFANYGLT

SEQ ID NO: 111 - protein, 8 aa, synthetic construct
GYTFANYG

SEQ ID NO: 112 - protein, 5 aa, synthetic construct
NYGLT

SEQ ID NO: 113 - protein, 7 aa, synthetic construct
GYTFANY

SEQ ID NO: 114 - protein, 6 aa, synthetic construct
ANYGLT

SEQ ID NO: 115 - protein, 10 aa, synthetic construct
GFTFSTYAMS

SEQ ID NO: 116 - protein, 8 aa, synthetic construct
GFTFSTYA

SEQ ID NO: 117 - protein, 5 aa, synthetic construct
TYAMS

SEQ ID NO: 118 - protein, 7 aa, synthetic construct
GFTFSTY

SEQ ID NO: 119 - protein, 6 aa, synthetic construct
STYAMS

SEQ ID NO: 120 - protein, 10 aa, synthetic construct
GYTFTTYWMH

SEQ ID NO: 121 - protein, 8 aa, synthetic construct
GYTFTTYW

SEQ ID NO: 122 - protein, 5 aa, synthetic construct
TYWMH

SEQ ID NO: 123 - protein, 6 aa, synthetic construct
TTYWMH

SEQ ID NO: 124 - protein, 10 aa, synthetic construct
GYTFTSYWMQ

SEQ ID NO: 125 - protein, 5 aa, synthetic construct
SYWMQ

SEQ ID NO: 126 - protein, 6 aa, synthetic construct
TSYWMQ

SEQ ID NO: 127 - protein, 11 aa, synthetic construct
GYSITSGYYWN

SEQ ID NO: 128 - protein, 9 aa, synthetic construct
GYSITSGYY

SEQ ID NO: 129 - protein, 6 aa, synthetic construct
SGYYWN

SEQ ID NO: 130 - protein, 8 aa, synthetic construct
GYSITSGY

SEQ ID NO: 131 - protein, 7 aa, synthetic construct
TSGYYWN

SEQ ID NO: 132 - protein, 16 aa, synthetic construct
VIWSGGRTDYNAAFIS

SEQ ID NO: 133 - protein, 7 aa, synthetic construct
IWSGGRT

SEQ ID NO: 134 - skipped

SEQ ID NO: 135 - protein, 12 aa, synthetic construct
WLGVIWSGGRTD

SEQ ID NO: 136 - protein, 9 aa, synthetic construct
VIWSGGRTD

SEQ ID NO: 137 - protein, 17 aa, synthetic construct
WINTYTGEPTYADDLKG

SEQ ID NO: 138 - protein, 8 aa, synthetic construct
INTYTGEP

SEQ ID NO: 139 - protein, 4 aa, synthetic construct
TYTG

SEQ ID NO: 140 - protein, 13 aa, synthetic construct
WMGWINTYTGEPT

SEQ ID NO: 141 - protein, 10 aa, synthetic construct
WINTYTGEPT

SEQ ID NO: 142 - protein, 17 aa, synthetic construct
WINTYSGVPTYADDFKG

SEQ ID NO: 143 - protein, 8 aa, synthetic construct
INTYSGVP

SEQ ID NO: 144 - protein, 4 aa, synthetic construct
TYSG

SEQ ID NO: 145 - protein, 13 aa, synthetic construct
WMGWINTYSGVPT

SEQ ID NO: 146 - protein, 10 aa, synthetic construct
WINTYSGVPT

SEQ ID NO: 147 - protein, 17 aa, synthetic construct
RIYPGNGNTYHNEKFKG

SEQ ID NO: 148 - protein, 8 aa, synthetic construct
IYPGNGNT

SEQ ID NO: 149 - protein, 4 aa, synthetic construct
PGNG

SEQ ID NO: 150 - protein, 13 aa, synthetic construct
WIARIYPGNGNTY

SEQ ID NO: 151 - protein, 10 aa, synthetic construct
RIYPGNGNTY

SEQ ID NO: 152 - protein, 17 aa, synthetic construct
EILLGSDSIHFNEKFKG

SEQ ID NO: 153 - protein, 8 aa, synthetic construct
ILLGSDSI

SEQ ID NO: 154 - protein, 4 aa, synthetic construct
LGSD

SEQ ID NO: 155 - protein, 13 aa, synthetic construct
WIGEILLGSDSIH

SEQ ID NO: 156 - protein, 10 aa, synthetic construct
EILLGSDSIH

SEQ ID NO: 157 - protein, 16 aa, synthetic construct
MIWGFGSTDYNSALKS

SEQ ID NO: 158 - skipped

SEQ ID NO: 159 - protein, 12 aa, synthetic construct
WLGMIWGFGSTD

SEQ ID NO: 160 - protein, 9 aa, synthetic construct
MIWGFGSTD

SEQ ID NO: 161 - protein, 17 aa, synthetic construct
YINPNSNYAEYNQKFKV

SEQ ID NO: 162 - protein, 8 aa, synthetic construct
INPNSNYA

SEQ ID NO: 163 - protein, 4 aa, synthetic construct
PNSN

SEQ ID NO: 164 - protein, 13 aa, synthetic construct
WIGYINPNSNYAE

SEQ ID NO: 165 - protein, 10 aa, synthetic construct
YINPNSNYAE

SEQ ID NO: 166 - protein, 17 aa, synthetic construct
RIYPGDGDTNYNGKFKG

SEQ ID NO: 167 - protein, 8 aa, synthetic construct
IYPGDGDT

SEQ ID NO: 168 - protein, 4 aa, synthetic construct
PGDG

SEQ ID NO: 169 - protein, 13 aa, synthetic construct
WIGRIYPGDGDTN

SEQ ID NO: 170 - protein, 10 aa, synthetic construct
RIYPGDGDTN

SEQ ID NO: 171 - protein, 17 aa, synthetic construct
WINTYSGVPTFVDDFRG

SEQ ID NO: 172 - protein, 17 aa, synthetic construct
WINTETGEPTYADDFKG

SEQ ID NO: 173 - protein, 8 aa, synthetic construct
INTETGEP

SEQ ID NO: 174 - protein, 4 aa, synthetic construct
TETG

SEQ ID NO: 175 - protein, 13 aa, synthetic construct
WMGWINTETGEPT

SEQ ID NO: 176 - protein, 10 aa, synthetic construct
WINTETGEPT

SEQ ID NO: 177 - protein, 9 aa, synthetic construct
WIFPGDGST

SEQ ID NO: 178 - protein, 8 aa, synthetic construct
IFPGDGST

SEQ ID NO: 179 - protein, 17 aa, synthetic construct
WIFPGDGSTKYNEKFKG

SEQ ID NO: 180 - protein, 13 aa, synthetic construct
WIGWIFPGDGSTK

SEQ ID NO: 181 - protein, 10 aa, synthetic construct
WIFPGDGSTK

SEQ ID NO: 182 - protein, 17 aa, synthetic construct
WIFPGDDSIKYNENFRG

SEQ ID NO: 183 - protein, 8 aa, synthetic construct
IFPGDDSI

SEQ ID NO: 184 - protein, 4 aa, synthetic construct
PGDD

SEQ ID NO: 185 - protein, 13 aa, synthetic construct
WIGWIFPGDDSIK

SEQ ID NO: 186 - protein, 10 aa, synthetic construct
WIFPGDDSIK

SEQ ID NO: 187 - protein, 17 aa, synthetic construct
WIFPGDDNAKYNEKFKG

SEQ ID NO: 188 - protein, 8 aa, synthetic construct
IFPGDDNA

SEQ ID NO: 189 - protein, 13 aa, synthetic construct
WIGWIFPGDDNAK

SEQ ID NO: 190 - protein, 10 aa, synthetic construct
WIFPGDDNAK

SEQ ID NO: 191 - protein, 17 aa, synthetic construct
YISNDGDSTYYPDTVQG

SEQ ID NO: 192 - protein, 8 aa, synthetic construct
ISNDGDST

SEQ ID NO: 193 - protein, 4 aa, synthetic construct
NDGD

SEQ ID NO: 194 - protein, 13 aa, synthetic construct
WVAYISNDGDSTY

SEQ ID NO: 195 - protein, 10 aa, synthetic construct
YISNDGDSTY

SEQ ID NO: 196 - protein, 17 aa, synthetic construct
EIYPGSGHTHYNEDFKG

SEQ ID NO: 197 - protein, 8 aa, synthetic construct
IYPGSGHT

SEQ ID NO: 198 - protein, 4 aa, synthetic construct
PGSG

SEQ ID NO: 199 - protein, 13 aa, synthetic construct
WIGEIYPGSGHTH

SEQ ID NO: 200 - protein, 10 aa, synthetic construct
EIYPGSGHTH

SEQ ID NO: 201 - protein, 16 aa, synthetic construct
SITSGGTTYYTDSVKG

SEQ ID NO: 202 - protein, 7 aa, synthetic construct
ITSGGTT

SEQ ID NO: 203 - protein, 12 aa, synthetic construct
WVASITSGGTTY

SEQ ID NO: 204 - protein, 9 aa, synthetic construct
SITSGGTTY

SEQ ID NO: 205 - protein, 17 aa, synthetic construct
MIHPNSGSSNYNEKFKN

SEQ ID NO: 206 - protein, 8 aa, synthetic construct
IHPNSGSS

SEQ ID NO: 207 - protein, 4 aa, synthetic construct
PNSG

SEQ ID NO: 208 - protein, 13 aa, synthetic construct
WIGMIHPNSGSSN

SEQ ID NO: 209 - protein, 10 aa, synthetic construct
MIHPNSGSSN

SEQ ID NO: 210 - protein, 17 aa, synthetic construct
EIDPSDSYTNYNQKFKG

SEQ ID NO: 211 - protein, 8 aa, synthetic construct
IDPSDSYT

SEQ ID NO: 212 - protein, 4 aa, synthetic construct
PSDS

SEQ ID NO: 213 - protein, 13 aa, synthetic construct
WIGEIDPSDSYTN

SEQ ID NO: 214 - protein, 10 aa, synthetic construct
EIDPSDSYTN

SEQ ID NO: 215 - protein, 16 aa, synthetic construct
HIANDGSNYYNPFLKH

SEQ ID NO: 216 - protein, 7 aa, synthetic construct
IANDGSN

SEQ ID NO: 217 - skipped

SEQ ID NO: 218 - protein, 12 aa, synthetic construct
WMGHIANDGSNY

SEQ ID NO: 219 - protein, 9 aa, synthetic construct
HIANDGSNY

SEQ ID NO: 220 - protein, 7 aa, synthetic construct
IWGFGST

SEQ ID NO: 221 - protein, 13 aa, synthetic construct
WALYFLYGGSMDY

SEQ ID NO: 222 - protein, 15 aa, synthetic construct
ARWALYFLYGGSMDY

SEQ ID NO: 223 - protein, 11 aa, synthetic construct
ALYFLYGGSMD

SEQ ID NO: 224 - protein, 14 aa, synthetic construct
ARWALYFLYGGSMD

SEQ ID NO: 225 - protein, 9 aa, synthetic construct
RYGPEDIDY

SEQ ID NO: 226 - protein, 11 aa, synthetic construct
ARRYGPEDIDY

SEQ ID NO: 227 - protein, 7 aa, synthetic construct
YGPEDID

SEQ ID NO: 228 - protein, 10 aa, synthetic construct
ARRYGPEDID

SEQ ID NO: 229 - protein, 5 aa, synthetic construct
ATGNY

SEQ ID NO: 230 - protein, 7 aa, synthetic construct
ASATGNY

SEQ ID NO: 231 - skipped

SEQ ID NO: 232 - protein, 6 aa, synthetic construct
ASATGN

SEQ ID NO: 233 - protein, 13 aa, synthetic construct
EGLYYDYDRYFDY

SEQ ID NO: 234 - protein, 15 aa, synthetic construct
AREGLYYDYDRYFDY

SEQ ID NO: 235 - protein, 11 aa, synthetic construct
GLYYDYDRYFD

SEQ ID NO: 236 - protein, 14 aa, synthetic construct
AREGLYYDYDRYFD

SEQ ID NO: 237 - protein, 9 aa, synthetic construct
QDWNWYFDV

SEQ ID NO: 238 - protein, 11 aa, synthetic construct
VRQDWNWYFDV

SEQ ID NO: 239 - protein, 7 aa, synthetic construct
DWNWYFD

SEQ ID NO: 240 - protein, 10 aa, synthetic construct
VRQDWNWYFD

SEQ ID NO: 241 - protein, 8 aa, synthetic construct
IHTTAGSY

SEQ ID NO: 242 - protein, 10 aa, synthetic construct
ARIHTTAGSY

SEQ ID NO: 243 - protein, 6 aa, synthetic construct
HTTAGS

SEQ ID NO: 244 - protein, 9 aa, synthetic construct
ARIHTTAGS

SEQ ID NO: 245 - protein, 9 aa, synthetic construct
FDWNWYFHV

SEQ ID NO: 246 - protein, 11 aa, synthetic construct
ARFDWNWYFHV

SEQ ID NO: 247 - protein, 7 aa, synthetic construct
DWNWYFH

SEQ ID NO: 248 - protein, 10 aa, synthetic construct
ARFDWNWYFH

SEQ ID NO: 249 - protein, 16 aa, synthetic construct
AYLLRLRRTGYYAMDY

SEQ ID NO: 250 - protein, 18 aa, synthetic construct
ARAYLLRLRRTGYYAMDY

SEQ ID NO: 251 - protein, 14 aa, synthetic construct
YLLRLRRTGYYAMD

SEQ ID NO: 252 - protein, 17 aa, synthetic construct
ARAYLLRLRRTGYYAMD

SEQ ID NO: 253 - protein, 12 aa, synthetic construct
RSSYYPYWYFDV

SEQ ID NO: 254 - protein, 14 aa, synthetic construct
ARRSSYYPYWYFDV

SEQ ID NO: 255 - protein, 10 aa, synthetic construct
SSYYPYWYFD

SEQ ID NO: 256 - protein, 13 aa, synthetic construct
ARRSSYYPYWYFD

SEQ ID NO: 257 - protein, 5 aa, synthetic construct
GTLNY

SEQ ID NO: 258 - protein, 7 aa, synthetic construct
VKGTLNY

SEQ ID NO: 259 - skipped

SEQ ID NO: 260 - protein, 6 aa, synthetic construct
VKGTLN

SEQ ID NO: 261 - protein, 11 aa, synthetic construct
SGIYYGSHFVY

SEQ ID NO: 262 - protein, 13 aa, synthetic construct
ARSGIYYGSHFVY

SEQ ID NO: 263 - protein, 9 aa, synthetic construct
GIYYGSHFV

SEQ ID NO: 264 - protein, 12 aa, synthetic construct
ARSGIYYGSHFV

SEQ ID NO: 265 - protein, 11 aa, synthetic construct
SGIFYGNNFAY

SEQ ID NO: 266 - protein, 13 aa, synthetic construct
ARSGIFYGNNFAY

SEQ ID NO: 267 - protein, 9 aa, synthetic construct
GIFYGNNFA

SEQ ID NO: 268 - protein, 12 aa, synthetic construct
ARSGIFYGNNFA

SEQ ID NO: 269 - protein, 10 aa, synthetic construct
TPVLSNYFDY

SEQ ID NO: 270 - protein, 12 aa, synthetic construct
ARTPVLSNYFDY

SEQ ID NO: 271 - protein, 8 aa, synthetic construct
PVLSNYFD

SEQ ID NO: 272 - protein, 11 aa, synthetic construct
ARTPVLSNYFD

SEQ ID NO: 273 - protein, 9 aa, synthetic construct
QGAQATLDY

SEQ ID NO: 274 - protein, 11 aa, synthetic construct
TRQGAQATLDY

SEQ ID NO: 275 - protein, 7 aa, synthetic construct
GAQATLD

SEQ ID NO: 276 - protein, 10 aa, synthetic construct
TRQGAQATLD

SEQ ID NO: 277 - protein, 13 aa, synthetic construct
RIQLLLPVGGFVY

SEQ ID NO: 278 - protein, 15 aa, synthetic construct
ARRIQLLLPVGGFVY

SEQ ID NO: 279 - protein, 11 aa, synthetic construct
IQLLLPVGGFV

SEQ ID NO: 280 - protein, 14 aa, synthetic construct
ARRIQLLLPVGGFV

SEQ ID NO: 281 - protein, 11 aa, synthetic construct
DGNFYYYGMDY

SEQ ID NO: 282 - protein, 13 aa, synthetic construct
ARDGNFYYYGMDY

SEQ ID NO: 283 - protein, 9 aa, synthetic construct
GNFYYYGMD

SEQ ID NO: 284 - protein, 12 aa, synthetic construct
ARDGNFYYYGMD

SEQ ID NO: 285 - protein, 11 aa, synthetic construct
SDYGFIPYFDY

SEQ ID NO: 286 - protein, 13 aa, synthetic construct
ARSDYGFIPYFDY

SEQ ID NO: 287 - protein, 9 aa, synthetic construct
DYGFIPYFD

SEQ ID NO: 288 - protein, 12 aa, synthetic construct
ARSDYGFIPYFD

SEQ ID NO: 289 - protein, 11 aa, synthetic construct
PLDRSAYYFDY

SEQ ID NO: 290 - protein, 13 aa, synthetic construct
ARPLDRSAYYFDY

SEQ ID NO: 291 - protein, 9 aa, synthetic construct
LDRSAYYFD

SEQ ID NO: 292 - protein, 12 aa, synthetic construct
ARPLDRSAYYFD

SEQ ID NO: 293 - protein, 10 aa, synthetic construct
GGSYFDYVDY

SEQ ID NO: 294 - protein, 12 aa, synthetic construct
ARGGSYFDYVDY

SEQ ID NO: 295 - protein, 8 aa, synthetic construct
GSYFDYVD

SEQ ID NO: 296 - protein, 11 aa, synthetic construct
ARGGSYFDYVD

SEQ ID NO: 297 - protein, 16 aa, synthetic construct
RSSQSLVHSSGITYLH

SEQ ID NO: 298 - protein, 11 aa, synthetic construct
QSLVHSSGITY

SEQ ID NO: 299 - protein, 12 aa, synthetic construct
SQSLVHSSGITY

SEQ ID NO: 300 - protein, 12 aa, synthetic construct
VHSSGITYLHWY

SEQ ID NO: 301 - protein, 15 aa, synthetic construct
RASESVDNYGISFMS

SEQ ID NO: 302 - protein, 10 aa, synthetic construct
ESVDNYGISF

SEQ ID NO: 303 - protein, 11 aa, synthetic construct
SESVDNYGISF

SEQ ID NO: 304 - protein, 11 aa, synthetic construct
DNYGISFMSWF

SEQ ID NO: 305 - protein, 11 aa, synthetic construct
RASQDIGSSLN

SEQ ID NO: 306 - protein, 6 aa, synthetic construct
QDIGSS

SEQ ID NO: 307 - protein, 7 aa, synthetic construct
SQDIGSS

SEQ ID NO: 308 - protein, 7 aa, synthetic construct
GSSLNWL

SEQ ID NO: 309 - protein, 15 aa, synthetic construct
RASESVDTYGNSFMH

SEQ ID NO: 310 - protein, 10 aa, synthetic construct
ESVDTYGNSF

SEQ ID NO: 311 - protein, 11 aa, synthetic construct
SESVDTYGNSF

SEQ ID NO: 312 - protein, 11 aa, synthetic construct
DTYGNSFMHWY

SEQ ID NO: 313 - protein, 16 aa, synthetic construct
KSSQSLLDFDGKTYLN

SEQ ID NO: 314 - protein, 11 aa, synthetic construct
QSLLDFDGKTY

SEQ ID NO: 315 - protein, 12 aa, synthetic construct
SQSLLDFDGKTY

SEQ ID NO: 316 - protein, 12 aa, synthetic construct
LDFDGKTYLNWY

SEQ ID NO: 317 - protein, 11 aa, synthetic construct
KASQNVGTNVA

SEQ ID NO: 318 - protein, 6 aa, synthetic construct
QNVGTN

SEQ ID NO: 319 - protein, 7 aa, synthetic construct
SQNVGTN

SEQ ID NO: 320 - protein, 7 aa, synthetic construct
GTNVAWY

SEQ ID NO: 321 - protein, 16 aa, synthetic construct
KSSQSLSDSDGKTYLN

SEQ ID NO: 322 - protein, 11 aa, synthetic construct
QSLSDSDGKTY

SEQ ID NO: 323 - protein, 12 aa, synthetic construct
SQSLSDSDGKTY

SEQ ID NO: 324 - protein, 12 aa, synthetic construct
SDSDGKTYLNWL

SEQ ID NO: 325 - protein, 16 aa, synthetic construct
KSTKSLLNSDEFTYLD

SEQ ID NO: 326 - protein, 11 aa, synthetic construct
KSLLNSDEFTY

SEQ ID NO: 327 - protein, 12 aa, synthetic construct
TKSLLNSDEFTY

SEQ ID NO: 328 - protein, 12 aa, synthetic construct
LNSDEFTYLDWY

SEQ ID NO: 329 - protein, 11 aa, synthetic construct
RPSENIYSYLT

SEQ ID NO: 330 - protein, 6 aa, synthetic construct
ENIYSY

SEQ ID NO: 331 - protein, 7 aa, synthetic construct
SENIYSY

SEQ ID NO: 332 - protein, 7 aa, synthetic construct
YSYLTWF

SEQ ID NO: 333 - protein, 11 aa, synthetic construct
KASQDIKSYLN

SEQ ID NO: 334 - protein, 6 aa, synthetic construct
QDIKSY

SEQ ID NO: 335 - protein, 7 aa, synthetic construct
SQDIKSY

SEQ ID NO: 336 - protein, 7 aa, synthetic construct
KSYLNWF

SEQ ID NO: 337 - protein, 16 aa, synthetic construct
RSSQSLLDSDGKTYLN

SEQ ID NO: 338 - protein, 11 aa, synthetic construct
QSLLDSDGKTY

SEQ ID NO: 339 - protein, 12 aa, synthetic construct
SQSLLDSDGKTY

SEQ ID NO: 340 - protein, 12 aa, synthetic construct
LDSDGKTYLNWL

SEQ ID NO: 341 - protein, 16 aa, synthetic construct
KSSQSLLDGDGETYLS

SEQ ID NO: 342 - protein, 11 aa, synthetic construct
QSLLDGDGETY

SEQ ID NO: 343 - protein, 12 aa, synthetic construct
SQSLLDGDGETY

SEQ ID NO: 344 - protein, 12 aa, synthetic construct
LDGDGETYLSWL

SEQ ID NO: 345 - protein, 11 aa, synthetic construct
KASQDISKYIS

SEQ ID NO: 346 - protein, 6 aa, synthetic construct
QDISKY

SEQ ID NO: 347 - protein, 7 aa, synthetic construct
SQDISKY

SEQ ID NO: 348 - protein, 7 aa, synthetic construct
SKYISWY

SEQ ID NO: 349 - protein, 10 aa, synthetic construct
SASSSVFYMH

SEQ ID NO: 350 - protein, 5 aa, synthetic construct
SSVFY

SEQ ID NO: 351 - protein, 6 aa, synthetic construct
SSSVFY

SEQ ID NO: 352 - protein, 6 aa, synthetic construct
FYMHWY

SEQ ID NO: 353 - protein, 11 aa, synthetic construct
RASQSISNNLH

SEQ ID NO: 354 - protein, 6 aa, synthetic construct
QSISNN

SEQ ID NO: 355 - protein, 7 aa, synthetic construct
SQSISNN

SEQ ID NO: 356 - protein, 7 aa, synthetic construct
SNNLHWY

SEQ ID NO: 357 - protein, 11 aa, synthetic construct
KASQNVGTAVA

SEQ ID NO: 358 - protein, 6 aa, synthetic construct
QNVGTA

SEQ ID NO: 359 - protein, 7 aa, synthetic construct
SQNVGTA

SEQ ID NO: 360 - protein, 7 aa, synthetic construct
GTAVAWY

SEQ ID NO: 361 - protein, 11 aa, synthetic construct
RASQSIGTSIH

SEQ ID NO: 362 - protein, 6 aa, synthetic construct
QSIGTS

SEQ ID NO: 363 - protein, 7 aa, synthetic construct
SQSIGTS

SEQ ID NO: 364 - protein, 7 aa, synthetic construct
GTSIHWY

SEQ ID NO: 365 - protein, 15 aa, synthetic construct
RASESVDFSGNSFMH

SEQ ID NO: 366 - protein, 10 aa, synthetic construct
ESVDFSGNSF

SEQ ID NO: 367 - protein, 11 aa, synthetic construct
SESVDFSGNSF

SEQ ID NO: 368 - protein, 11 aa, synthetic construct
DFSGNSFMHWY

SEQ ID NO: 369 - protein, 11 aa, synthetic construct
RASQDISNYLN

SEQ ID NO: 370 - protein, 6 aa, synthetic construct
QDISNY

SEQ ID NO: 371 - protein, 7 aa, synthetic construct
SQDISNY

SEQ ID NO: 372 - protein, 7 aa, synthetic construct
SNYLNWY

SEQ ID NO: 373 - protein, 7 aa, synthetic construct
KLSNRFS

SEQ ID NO: 374 - skipped

SEQ ID NO: 375 - protein, 10 aa, synthetic construct
LLIYKLSNRF

SEQ ID NO: 376 - protein, 7 aa, synthetic construct
AASHQGS

SEQ ID NO: 377 - skipped

SEQ ID NO: 378 - protein, 10 aa, synthetic construct
LLIYAASHQG

SEQ ID NO: 379 - protein, 7 aa, synthetic construct
ATSSLDS

SEQ ID NO: 380 - skipped

SEQ ID NO: 381 - protein, 10 aa, synthetic construct
RLIYATSSLD

SEQ ID NO: 382 - protein, 7 aa, synthetic construct
LASNLES

SEQ ID NO: 383 - skipped

SEQ ID NO: 384 - protein, 10 aa, synthetic construct
LLIYLASNLE

SEQ ID NO: 385 - protein, 7 aa, synthetic construct
LVSKLDS

SEQ ID NO: 386 - skipped

SEQ ID NO: 387 - protein, 10 aa, synthetic construct
RLFYLVSKLD

SEQ ID NO: 388 - protein, 7 aa, synthetic construct
STSYRYS

SEQ ID NO: 389 - skipped

SEQ ID NO: 390 - protein, 10 aa, synthetic construct
ALVYSTSYRY

SEQ ID NO: 391 - protein, 7 aa, synthetic construct
LVSRLGS

SEQ ID NO: 392 - protein, 10 aa, synthetic construct
RLIYLVSRLG

SEQ ID NO: 393 - protein, 7 aa, synthetic construct
LVSNRFS

SEQ ID NO: 394 - protein, 10 aa, synthetic construct
LLIFLVSNRF

SEQ ID NO: 395 - protein, 7 aa, synthetic construct
NAQTLAE

SEQ ID NO: 396 - skipped

SEQ ID NO: 397 - protein, 10 aa, synthetic construct
LLVYNAQTLA

SEQ ID NO: 398 - protein, 7 aa, synthetic construct
RTKRLVD

SEQ ID NO: 399 - skipped

SEQ ID NO: 400 - protein, 10 aa, synthetic construct
TLIYRTKRLV

SEQ ID NO: 401 - protein, 7 aa, synthetic construct
LVSKVDS

SEQ ID NO: 402 - protein, 10 aa, synthetic construct
RLIYLVSKVD

SEQ ID NO: 403 - protein, 10 aa, synthetic construct
RLIYLVSKLD

SEQ ID NO: 404 - protein, 7 aa, synthetic construct
YTSTLQP

SEQ ID NO: 405 - skipped

SEQ ID NO: 406 - protein, 10 aa, synthetic construct
LLIHYTSTLQ

SEQ ID NO: 407 - protein, 10 aa, synthetic construct
LLIYLVSNRF

SEQ ID NO: 408 - protein, 7 aa, synthetic construct
STSNLAS

SEQ ID NO: 409 - protein, 10 aa, synthetic construct
LLIYSTSNLA

SEQ ID NO: 410 - protein, 7 aa, synthetic construct
YASQSIS

SEQ ID NO: 411 - skipped

SEQ ID NO: 412 - protein, 10 aa, synthetic construct
LLIKYASQSI

SEQ ID NO: 413 - protein, 7 aa, synthetic construct
SASNRFT

SEQ ID NO: 414 - skipped

SEQ ID NO: 415 - protein, 10 aa, synthetic construct
ILIYSASNRF

SEQ ID NO: 416 - protein, 7 aa, synthetic construct
YASESIS

SEQ ID NO: 417 - protein, 10 aa, synthetic construct
LLIKYASESI

SEQ ID NO: 418 - protein, 7 aa, synthetic construct
RASNLDS

SEQ ID NO: 419 - skipped

SEQ ID NO: 420 - protein, 10 aa, synthetic construct
LLIYRASNLD

SEQ ID NO: 421 - protein, 7 aa, synthetic construct
YTSRLHS

SEQ ID NO: 422 - protein, 10 aa, synthetic construct
LLIYYTSRLH

SEQ ID NO: 423 - protein, 10 aa, synthetic construct
SQSTHVPPWT

SEQ ID NO: 424 - protein, 7 aa, synthetic construct
STHVPPW

SEQ ID NO: 425 - protein, 9 aa, synthetic construct
SQSTHVPPW

SEQ ID NO: 426 - protein, 9 aa, synthetic construct
LQSKEVPWT

SEQ ID NO: 427 - protein, 6 aa, synthetic construct
SKEVPW

SEQ ID NO: 428 - protein, 8 aa, synthetic construct
LQSKEVPW

SEQ ID NO: 429 - protein, 9 aa, synthetic construct
LQYASSPYT

SEQ ID NO: 430 - protein, 6 aa, synthetic construct
YASSPY

SEQ ID NO: 431 - protein, 8 aa, synthetic construct
LQYASSPY

SEQ ID NO: 432 - protein, 9 aa, synthetic construct
HQNNEDPPA

SEQ ID NO: 433 - protein, 6 aa, synthetic construct
NNEDPP

SEQ ID NO: 434 - protein, 8 aa, synthetic construct
HQNNEDPP

SEQ ID NO: 435 - protein, 9 aa, synthetic construct
WQGTHFPRT

SEQ ID NO: 436 - protein, 6 aa, synthetic construct
GTHFPR

SEQ ID NO: 437 - protein, 8 aa, synthetic construct
WQGTHFPR

SEQ ID NO: 438 - protein, 9 aa, synthetic construct
HQYNSYPLT

SEQ ID NO: 439 - protein, 6 aa, synthetic construct
YNSYPL

SEQ ID NO: 440 - protein, 8 aa, synthetic construct
HQYNSYPL

SEQ ID NO: 441 - protein, 9 aa, synthetic construct
WQGTHFPQT

SEQ ID NO: 442 - protein, 6 aa, synthetic construct
GTHFPQ

SEQ ID NO: 443 - protein, 8 aa, synthetic construct
WQGTHFPQ

SEQ ID NO: 444 - protein, 9 aa, synthetic construct
FQSNYLPYT

SEQ ID NO: 445 - protein, 6 aa, synthetic construct
SNYLPY

SEQ ID NO: 446 - protein, 8 aa, synthetic construct
FQSNYLPY

SEQ ID NO: 447 - protein, 9 aa, synthetic construct
QHYYGYPFT

SEQ ID NO: 448 - protein, 6 aa, synthetic construct
YYGYPF

SEQ ID NO: 449 - protein, 8 aa, synthetic construct
QHYYGYPF

SEQ ID NO: 450 - protein, 9 aa, synthetic construct
LQYVEFPLT

SEQ ID NO: 451 - protein, 6 aa, synthetic construct
YVEFPL

SEQ ID NO: 452 - protein, 8 aa, synthetic construct
LQYVEFPL

SEQ ID NO: 453 - protein, 9 aa, synthetic construct
WQGTHFPLT

SEQ ID NO: 454 - protein, 6 aa, synthetic construct
GTHFPL

SEQ ID NO: 455 - protein, 8 aa, synthetic construct
WQGTHFPL

SEQ ID NO: 456 - protein, 9 aa, synthetic construct
CQSTHFPLT

SEQ ID NO: 457 - protein, 6 aa, synthetic construct
STHFPL

SEQ ID NO: 458 - protein, 8 aa, synthetic construct
CQSTHFPL

SEQ ID NO: 459 - protein, 8 aa, synthetic construct
LQYDNLYT

SEQ ID NO: 460 - protein, 5 aa, synthetic construct
YDNLY

SEQ ID NO: 461 - protein, 7 aa, synthetic construct
LQYDNLY

SEQ ID NO: 462 - protein, 6 aa, synthetic construct
HQWSST

SEQ ID NO: 463 - skipped

SEQ ID NO: 464 - protein, 5 aa, synthetic construct
HQWSS

SEQ ID NO: 465 - protein, 9 aa, synthetic construct
QQSNSWPHT

SEQ ID NO: 466 - protein, 6 aa, synthetic construct
SNSWPH

SEQ ID NO: 467 - protein, 8 aa, synthetic construct
QQSNSWPH

SEQ ID NO: 468 - protein, 8 aa, synthetic construct
QQYSSYFT

SEQ ID NO: 469 - protein, 5 aa, synthetic construct
YSSYF

SEQ ID NO: 470 - protein, 7 aa, synthetic construct
QQYSSYF

SEQ ID NO: 471 - protein, 10 aa, synthetic construct
QQSNSWPTFT

SEQ ID NO: 472 - protein, 7 aa, synthetic construct
SNSWPTF

SEQ ID NO: 473 - protein, 9 aa, synthetic construct
QQSNSWPTF

SEQ ID NO: 474 - protein, 9 aa, synthetic construct
QQSNEDPYT

SEQ ID NO: 475 - protein, 6 aa, synthetic construct
SNEDPY

SEQ ID NO: 476 - protein, 8 aa, synthetic construct
QQSNEDPY

SEQ ID NO: 477 - protein, 9 aa, synthetic construct
QQGNTLPFT

SEQ ID NO: 478 - protein, 6 aa, synthetic construct
GNTLPF

SEQ ID NO: 479 - protein, 8 aa, synthetic construct
QQGNTLPF

SEQ ID NO: 480 - protein, 118 aa, synthetic construct
EVQLQQSGPELVKPGASVKISCKASGFTFTDYYMNWVKQSHGKSLEWIGDIIPNNGVTSYNQKFKGKATLTVDKSSSTAYMELRSLTSEDSAVYYCAREWLLRGMDYWGQGTSVTVSS

SEQ ID NO: 481 - protein, 112 aa, synthetic construct
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQHLELTWTFGGGTKLEIK

SEQ ID NO: 482 - protein, 117 aa, synthetic construct
EVQLQQSGPELVKPGASVKMSCKASGYTFTDYYMNWVKQTHGKSLEWIGDINPNNGGPIYNQKFKGKATLTVDKSSSTAYMELRSLTSEDSAVYYCARSDSAWFTYWGQGTLVTVSA

SEQ ID NO: 483 - protein, 106 aa, synthetic construct
QIVLTQSPAIMSASPGEKVTMTCSASSSVSYMYWYQQKPGSSPRLLIYDTSNLASGVPVRFSGSGSGTFYSITISRMEAEDAATYYCQQWNSYPPTFGGGTKLEIK

SEQ ID NO: 484 - protein, 117 aa, synthetic construct
EVQLQQSGPELVKPGASVKISCKASGYTFTDYYMNWVKQSHGKSLEWIGDINPNNGGTTYNQKFKGKATLTVDKSSSTAYMELRSLTSEDSAVYYCARQGPWYFDVWGTGTTVTVSS

SEQ ID NO: 485 - protein, 112 aa, synthetic construct
DVLMTQTPLSLPVSLGDQASISCRSSQTIVHSNGYTYLEWYLQKPGQSPKLLIYKVSNRFSGVPDRFSGSGSGTDFTLKISRVEAEDLGVYYCFQGSHVPWTFGGGTKLEIK

SEQ ID NO: 486 - protein, 113 aa, synthetic construct
QVQLKQSGPGLVQPSQSLSITCTVSGFSLTSYGVHWVRQSPGKGLDWLGVLWSGGSTDYNAAFISRLSISKDNSKSQVFFKMNSLQADDTAIYYCARNFGDYWGQGTSVTVSS

SEQ ID NO: 487 - protein, 106 aa, synthetic construct
QIVLTQSPAIMSASPGEKVTMTCSASSRVSYMHWYQQKSGTSPKRWIYDTSKLASGVPARFSGSGSGTSYSLTISSMEAEDAATYYCQQWNNNPPTFGAGTTLELK

SEQ ID NO: 488 - protein, 10 aa, synthetic construct
GFTFTDYYMN

SEQ ID NO: 489 - protein, 10 aa, synthetic construct
GYTFTDYYMN

SEQ ID NO: 490 - protein, 5 aa, synthetic construct
DYYMN

SEQ ID NO: 491 - protein, 6 aa, synthetic construct
TDYYMN

SEQ ID NO: 492 - protein, 10 aa, synthetic construct
GFSLTSYGVH

SEQ ID NO: 493 - protein, 8 aa, synthetic construct
GFSLTSYG

SEQ ID NO: 494 - protein, 5 aa, synthetic construct
SYGVH

SEQ ID NO: 495 - protein, 7 aa, synthetic construct
GFSLTSY

SEQ ID NO: 496 - protein, 6 aa, synthetic construct
TSYGVH

SEQ ID NO: 497 - protein, 17 aa, synthetic construct
DIIPNNGVTSYNQKFKG

SEQ ID NO: 498 - protein, 8 aa, synthetic construct
IIPNNGVT

SEQ ID NO: 499 - protein, 4 aa, synthetic construct
PNNG

SEQ ID NO: 500 - protein, 13 aa, synthetic construct
WIGDIIPNNGVTS

SEQ ID NO: 501 - protein, 10 aa, synthetic construct
DIIPNNGVTS

SEQ ID NO: 502 - protein, 17 aa, synthetic construct
DINPNNGGPIYNQKFKG

SEQ ID NO: 503 - protein, 8 aa, synthetic construct
INPNNGGP

SEQ ID NO: 504 - protein, 13 aa, synthetic construct
WIGDINPNNGGPI

SEQ ID NO: 505 - protein, 10 aa, synthetic construct
DINPNNGGPI

SEQ ID NO: 506 - protein, 17 aa, synthetic construct
DINPNNGGTTYNQKFKG

SEQ ID NO: 507 - protein, 8 aa, synthetic construct
INPNNGGT

SEQ ID NO: 508 - protein, 13 aa, synthetic construct
WIGDINPNNGGTT

SEQ ID NO: 509 - protein, 10 aa, synthetic construct
DINPNNGGTT

SEQ ID NO: 510 - protein, 16 aa, synthetic construct
VLWSGGSTDYNAAFIS

SEQ ID NO: 511 - protein, 7 aa, synthetic construct
LWSGGST

SEQ ID NO: 512 - skipped

SEQ ID NO: 513 - protein, 12 aa, synthetic construct
WLGVLWSGGSTD

SEQ ID NO: 514 - protein, 9 aa, synthetic construct
VLWSGGSTD

SEQ ID NO: 515 - protein, 9 aa, synthetic construct
EWLLRGMDY

SEQ ID NO: 516 - protein, 11 aa, synthetic construct
AREWLLRGMDY

SEQ ID NO: 517 - protein, 7 aa, synthetic construct
WLLRGMD

SEQ ID NO: 518 - protein, 10 aa, synthetic construct
AREWLLRGMD

SEQ ID NO: 519 - protein, 8 aa, synthetic construct
SDSAWFTY

SEQ ID NO: 520 - protein, 10 aa, synthetic construct
ARSDSAWFTY

SEQ ID NO: 521 - protein, 6 aa, synthetic construct
DSAWFT

SEQ ID NO: 522 - protein, 9 aa, synthetic construct
ARSDSAWFT

SEQ ID NO: 523 - protein, 8 aa, synthetic construct
QGPWYFDV

SEQ ID NO: 524 - protein, 10 aa, synthetic construct
ARQGPWYFDV

SEQ ID NO: 525 - protein, 6 aa, synthetic construct
GPWYFD

SEQ ID NO: 526 - protein, 9 aa, synthetic construct
ARQGPWYFD

SEQ ID NO: 527 - protein, 5 aa, synthetic construct
NFGDY

SEQ ID NO: 528 - protein, 7 aa, synthetic construct
ARNFGDY

SEQ ID NO: 529 - skipped

SEQ ID NO: 530 - protein, 6 aa, synthetic construct
ARNFGD

SEQ ID NO: 531 - protein, 16 aa, synthetic construct
RSSKSLLHSNGITYLY

SEQ ID NO: 532 - protein, 11 aa, synthetic construct
KSLLHSNGITY

SEQ ID NO: 533 - protein, 12 aa, synthetic construct
SKSLLHSNGITY

SEQ ID NO: 534 - protein, 12 aa, synthetic construct
LHSNGITYLYWY

SEQ ID NO: 535 - protein, 10 aa, synthetic construct
SASSSVSYMY

SEQ ID NO: 536 - protein, 5 aa, synthetic construct
SSVSY

SEQ ID NO: 537 - protein, 6 aa, synthetic construct
SSSVSY

SEQ ID NO: 538 - protein, 6 aa, synthetic construct
SYMYWY

SEQ ID NO: 539 - protein, 16 aa, synthetic construct
RSSQTIVHSNGYTYLE

SEQ ID NO: 540 - protein, 11 aa, synthetic construct
QTIVHSNGYTY

SEQ ID NO: 541 - protein, 12 aa, synthetic construct
SQTIVHSNGYTY

SEQ ID NO: 542 - protein, 12 aa, synthetic construct
VHSNGYTYLEWY

SEQ ID NO: 543 - protein, 10 aa, synthetic construct
SASSRVSYMH

SEQ ID NO: 544 - protein, 5 aa, synthetic construct
SRVSY

SEQ ID NO: 545 - protein, 6 aa, synthetic construct
SSRVSY

SEQ ID NO: 546 - protein, 6 aa, synthetic construct
SYMHWY

SEQ ID NO: 547 - protein, 7 aa, synthetic construct
QMSNLAS

SEQ ID NO: 548 - skipped

SEQ ID NO: 549 - protein, 10 aa, synthetic construct
LLIYQMSNLA

SEQ ID NO: 550 - protein, 7 aa, synthetic construct
DTSNLAS

SEQ ID NO: 551 - skipped

SEQ ID NO: 552 - protein, 10 aa, synthetic construct
LLIYDTSNLA

SEQ ID NO: 553 - protein, 7 aa, synthetic construct
KVSNRFS

SEQ ID NO: 554 - skipped

SEQ ID NO: 555 - protein, 10 aa, synthetic construct
LLIYKVSNRF

SEQ ID NO: 556 - protein, 7 aa, synthetic construct
DTSKLAS

SEQ ID NO: 557 - protein, 10 aa, synthetic construct
RWIYDTSKLA

SEQ ID NO: 558 - protein, 9 aa, synthetic construct
AQHLELTWT

SEQ ID NO: 559 - protein, 6 aa, synthetic construct
HLELTW

SEQ ID NO: 560 - protein, 8 aa, synthetic construct
AQHLELTW

SEQ ID NO: 561 - protein, 9 aa, synthetic construct
QQWNSYPPT

SEQ ID NO: 562 - protein, 6 aa, synthetic construct
WNSYPP

SEQ ID NO: 563 - protein, 8 aa, synthetic construct
QQWNSYPP

SEQ ID NO: 564 - protein, 9 aa, synthetic construct
FQGSHVPWT

SEQ ID NO: 565 - protein, 7 aa, synthetic construct
QGSHVPW

SEQ ID NO: 566 - protein, 8 aa, synthetic construct
FQGSHVPW

SEQ ID NO: 567 - protein, 9 aa, synthetic construct
QQWNNNPPT

SEQ ID NO: 568 - protein, 6 aa, synthetic construct
WNNNPP

SEQ ID NO: 569 - protein, 8 aa, synthetic construct
QQWNNNPP

SEQ ID NO: 570-1794 - skipped (1225 entries)

SEQ ID NO: 1795 - protein, 8 aa, synthetic construct
GFTFTDYY

SEQ ID NO: 1796 - protein, 7 aa, synthetic construct
GFTFTDY

SEQ ID NO: 1797 - protein, 394 aa, Homo sapiens
MIVFIFLAMGLSLENEYTSQTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEVIYAAMCMTVTCGILLLVMVKLRTSRISSKARDPSSIQIPGEL

SEQ ID NO: 1798 - protein, 375 aa, Homo sapiens
QTNNCTYLREQCLRDANGCKHAWRVMEDACNDSDPGDPCKMRNSSYCNLSIQYLVESNFQFKECLCTDDFYCTVNKLLGKKCINKSDNVKEDKFKWNLTTRSHHGFKGMWSCLEVAEACVGDVVCNAQLASYLKACSANGNPCDLKQCQAAIRFFYQNIPFNIAQMLAFCDCAQSDIPCQQSKEALHSKTCAVNMVPPPTCLSVIRSCQNDELCRRHYRTFQSKCWQRVTRKCHEDENCISTLSKQDLTCSGSDDCKAAYIDILGTVLQVQCTCRTITQSEESLCKIFQHMLHRKSCFNYPTLSNVKGMALYTRKHANKITLTGFHSPFNGEVIYAAMCMTVTCGILLLVMVKLRTSRISSKARDPSSIQIPGEL

SEQ ID NO: 1799-1809 - skipped (11 entries)

SEQ ID NO: 1810 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 1811 - protein, 112 aa, Homo sapiens
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 1812 - protein, 1049 aa, Homo sapiens
KVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 1813 - protein, 1072 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGRISHAFTRF

SEQ ID NO: 1814-1917 - skipped (104 entries)

SEQ ID NO: 1918 - protein, 121 aa, synthetic construct
QMQLKQSGPGLVQPSQSLSITCTVSGFSLNDYGVHWIRQSPGKGLEWLGVIWSGGRTDYNAAFISRLSISKDNSKSQVFFKMSSLQPNDTAIYYCARWALYFLYGGSMDYWGQGTSVTVSS

SEQ ID NO: 1919 - protein, 121 aa, synthetic construct
QVQLQESGPGLVKPSETLSLTCTVSGFSLNDYGVHWIRQPPGKGLEWIGVIWSGGRTDYNAAFISRVTISVDTSKNQFSLKLSSVTAADTAVYYCARWALYFLYGGSMDYWGQGTLVTVSS

SEQ ID NO: 1920 - protein, 121 aa, synthetic construct
QVQLQESGPGLVKPSETLSLTCTVSGFSLNDYGVHWIRQPPGKGLEWIGVIWSGGRTDYNAAFISRVTISKDTSKNQFSLKLSSVTAADTAVYYCARWALYFLYGGSMDYWGQGTLVTVSS

SEQ ID NO: 1921 - protein, 121 aa, synthetic construct
QMQLQESGPGLVKPSETLSLTCTVSGFSLNDYGVHWIRQPPGKGLEWIGVIWSGGRTDYNAAFISRLTISVDTSKNQVSLKLSSVQAADTAVYYCARWALYFLYGGSMDYWGQGTLVTVSS

SEQ ID NO: 1922 - protein, 121 aa, synthetic construct
QMQLQESGPGLVKPSETLSLTCTVSGFSLNDYGVHWIRQPPGKGLEWIGVIWSGGRTDYNAAFISRVTISKDNSKSQFSLKLSSVTAQDTAVYYCARWALYFLYGGSMDYWGQGTLVTVSS

SEQ ID NO: 1923 - protein, 121 aa, synthetic construct
QMQLQESGPGLVKPSETLSLTCTVSGFSLNDYGVHWIRQPPGKGLEWIGVIWSGGRTDYNAAFISRLTISKDTSKNQFSLKMSSVTAADTAVYYCARWALYFLYGGSMDYWGQGTLVTVSS

SEQ ID NO: 1924 - protein, 121 aa, synthetic construct
QMQLQESGPGLVKPSETLSLTCTVSGFSLNDYGVHWIRQPPGKGLEWLGVIWSGGRTDYNAAFISRLTISKDTSKNQVSFKMSSLTAADTAVYYCARWALYFLYGGSMDYWGQGTLVTVSS

SEQ ID NO: 1925 - protein, 121 aa, synthetic construct
QVQLQESGPGLVKPSETLSLTCTVSGFSLNDYGVHWIRQPPGKGLEWIGVIWSGGRTDYNAAFISRVTISRDTSKNQFSLKLSSVTAADTAVYYCARWALYFLYGGSMDYWGQGTLVTVSS

SEQ ID NO: 1926 - protein, 121 aa, synthetic construct
QMQLQESGPGLVKPSETLSLTCTVSGFSLNDYGVHWIRQPPGKGLEWIGVIWSGGRTDYNAAFISRVTISRDTSKSQFSLKLSSVTAADTAVYYCARWALYFLYGGSMDYWGQGTLVTVSS

SEQ ID NO: 1927 - protein, 121 aa, synthetic construct
QMQLQESGPGLVKPSETLSLTCTVSGFSLNDYGVHWIRQPPGKGLEWIGVIWSGGRTDYNAAFISRLTISRDTSKSQVSFKLSSVTAADTAVYYCARWALYFLYGGSMDYWGQGTLVTVSS

SEQ ID NO: 1928 - protein, 113 aa, synthetic construct; identical to SEQ ID NO: 2

SEQ ID NO: 1929 - protein, 113 aa, synthetic construct
DVVMTQSPLSLPVTLGQPASISCRSSQSLVHSSGITYLHWFQQRPGQSPRRLIYKLSNRFSGVPDRFSGSGSGTDFTLKISRVEAEDVGVYYCSQSTHVPPWTFGGGTKVEIK

SEQ ID NO: 1930 - protein, 113 aa, synthetic construct
DVVLTQSPLSLPVTLGQPASISCRSSQSLVHSSGITYLHWYQQRPGQSPRRLIYKLSNRFSGVPDRFSGSGSGTDFTLKISRVEAEDVGVYYCSQSTHVPPWTFGGGTKVEIK

SEQ ID NO: 1931 - protein, 113 aa, synthetic construct
DVVLTQSPLSLPVTLGQPASISCRSSQSLVHSSGITYLHWYQQRPGQSPKLLIYKLSNRFSGVPDRFSGSGSGTDFTLKISRVEAEDVGVYYCSQSTHVPPWTFGGGTKVEIK

SEQ ID NO: 1932 - protein, 113 aa, synthetic construct
DVVLTQSPLSLPVTLGDPASISCRSSQSLVHSSGITYLHWYQQKPGQSPKLLIYKLSNRFSGVPDRFSGSGSGTDFTLKISRVEAEDVGVYYCSQSTHVPPWTFGGGTKVEIK

SEQ ID NO: 1933 - protein, 113 aa, synthetic construct; identical to SEQ ID NO: 2

SEQ ID NO: 1934 - protein, 113 aa, synthetic construct
DIVMTQTPLSLPVSPGDQASISCRSSQSLVHSSGITYLHWYLQKPGQSPKLLIYKLSNRFSGVPDRFSGSGSGTDFTLKISRVEAEDLGVYFCSQSTHVPPWTFGGGTKLEIK

SEQ ID NO: 1935 - protein, 125 aa, synthetic construct; identical to SEQ ID NO: 15

SEQ ID NO: 1936 - protein, 125 aa, synthetic construct
EVQLVQSGAEVKKPGESLKISCKGSGYTFTSYWVNWVRQMPGKGLEWMGRIYPGDGDTNYNGKFKGQVTISADKSISTAYLQWSSLKASDTAMYYCARAYLLRLRRTGYYAMDYWGQGTTVTVSS

SEQ ID NO: 1937 - protein, 125 aa, synthetic construct
EVQLVQSGAEVKKPGESLKISCKGSGYTFTSYWVNWVRQMPGKGLEWIGRIYPGDGDTNYNGKFKGQATISADKSISTAYLQWSSLKASDTAMYYCARAYLLRLRRTGYYAMDYWGQGTTVTVSS

SEQ ID NO: 1938 - protein, 125 aa, synthetic construct
EVQLVQSGAEVKKPGESLKISCKGSGYTFTSYWVNWVRQMPGKGLEWIGRIYPGDGDTNYNGKFKGQATLSADKSISTAYLQWSSLKASDTAMYYCARAYLLRLRRTGYYAMDYWGQGTTVTVSS

SEQ ID NO: 1939 - protein, 125 aa, synthetic construct
QVQLVQSGAEVKKPGSSVKVSCKASGYTFTSYWVNWVRQAPGQGLEWMGRIYPGDGDTNYNGKFKGRVTITADESTSTAYMELSSLRSEDTAVYYCARAYLLRLRRTGYYAMDYWGQGTTVTVSS

SEQ ID NO: 1940 - protein, 125 aa, synthetic construct
QVQLVQSGAEVKKPGSSVKVSCKASGYTFTSYWVNWVRQAPGQGLEWIGRIYPGDGDTNYNGKFKGRATITADESTSTAYMELSSLRSEDTAVYYCARAYLLRLRRTGYYAMDYWGQGTTVTVSS

SEQ ID NO: 1941 - protein, 125 aa, synthetic construct
QVQLVQSGAEVKKPGSSVKVSCKASGYTFTSYWVNWVRQAPGQGLEWIGRIYPGDGDTNYNGKFKGRATLTADKSTSTAYMELSSLRSEDTAVYYCARAYLLRLRRTGYYAMDYWGQGTTVTVSS

SEQ ID NO: 1942 - protein, 112 aa, synthetic construct; identical to SEQ ID NO: 16

SEQ ID NO: 1943 - protein, 112 aa, synthetic construct
DVVMTQSPLSLPVTLGQPASISCKSTKSLLNSDEFTYLDWFQQRPGQSPRRLIYLVSNRFSGVPDRFSGSGSGTDFTLKISRVEAEDVGVYYCFQSNYLPYTFGGGTKVEIK

SEQ ID NO: 1944 - protein, 112 aa, synthetic construct
DVVLTQSPLSLPVTLGQPASISCKSTKSLLNSDEFTYLDWFQQRPGQSPRLLIYLVSNRFSGVPDRFSGSGSGTDFTLKISRVEAEDVGVYYCFQSNYLPYTFGGGTKVEIK

SEQ ID NO: 1945 - protein, 112 aa, synthetic construct
DVVLTQSPLSLPVTLGQPASISCKSTKSLLNSDEFTYLDWYQQRPGQSPRLLIFLVSNRFSGVPDRFSGSGSGTDFTLKISRVEAEDVGVYYCFQSNYLPYTFGGGTKVEIK

SEQ ID NO: 1946 - protein, 112 aa, synthetic construct
EIVLTQSPGTLSLSPGERATLSCKSTKSLLNSDEFTYLDWYQQKPGQAPRLLIYLVSNRFSGIPDRFSGSGSGTDFTLTISRLEPEDFAVYYCFQSNYLPYTFGGGTKVEIK

SEQ ID NO: 1947 - protein, 112 aa, synthetic construct
EVVLTQSPGTLSLSPGERATLSCKSTKSLLNSDEFTYLDWYQQKPGQAPRLLIFLVSNRFSGIPDRFSGSGSGTDFTLTISRLEPEDFAVYYCFQSNYLPYTFGGGTKVEIK

SEQ ID NO: 1948 - protein, 118 aa, synthetic construct; identical to SEQ ID NO: 13

SEQ ID NO: 1949 - protein, 118 aa, synthetic construct
QVQLVQSGAEVKKPGSSVKVSCKASGYTFTDYWIHWVRQAPGQGLEWMGYINPNSNYAEYNQKFKVRVTITADESTSTAYMELSSLRSEDTAVYYCARFDWNWYFHVWGQGTTVTVSS

SEQ ID NO: 1950 - protein, 118 aa, synthetic construct
QVQLVQSGAEVKKPGSSVKVSCKASGYTFTDYWIHWVRQAPGQGLEWIGYINPNSNYAEYNQKFKVRVTITADESTSTAYMELSRLRSEDTAVYYCARFDWNWYFHVWGQGTTVTVSS

SEQ ID NO: 1951 - protein, 118 aa, synthetic construct
QVQLVQSGAEVKKPGSSVKVSCKASGYTFTDYWIHWVRQAPGQGLEWIGYINPNSNYAEYNQKFKVRATLTADESTSTAYLELSRLRSEDTAVYYCARFDWNWYFHVWGQGTTVTVSS

SEQ ID NO: 1952 - protein, 118 aa, synthetic construct
QVQLVQSGAEVKKPGSSVKVSCKASGYTFTDYWIHWVKQAPGQGLEWIGYINPNSNYAEYNQKFKVRATLTADESSSTAYLELSRLRSEDTAVYYCARFDWNWYFHVWGQGTTVTVSS

SEQ ID NO: 1953 - protein, 112 aa, synthetic construct; identical to SEQ ID NO: 14

SEQ ID NO: 1954 - protein, 112 aa, synthetic construct
DVVMTQSPLSLPVTLGQPASISCKSSQSLSDSDGKTYLNWFQQRPGQSPRRLIYLVSRLGSGVPDRFSGSGSGTDFTLKISRVEAEDVGVYYCWQGTHFPQTFGGGTKVEIK

SEQ ID NO: 1955 - protein, 112 aa, synthetic construct
DVVLTQSPLSLPVTLGQPASISCKSSQSLSDSDGKTYLNWLQQRPGQSPRRLIYLVSRLGSGVPDRFSGSGSGTDFTLKISRVEAEDVGVYYCWQGTHFPQTFGGGTKVEIK

SEQ ID NO: 1956 - protein, 112 aa, synthetic construct
DVALTQSPLSLPVTLGQPASISCKSSQSLSDSDGKTYLNWLQQRPGQSPKRLIYLVSRLGSGVPDRFSGSGSGADFTLKISRVEAEDVGVYYCWQGTHFPQTFGGGTKVEIK

SEQ ID NO: 1957 - protein, 122 aa, synthetic construct; identical to SEQ ID NO: 7

SEQ ID NO: 1958 - protein, 122 aa, synthetic construct
QVQLVQSGAEVKKPGSSVKVSCKASGYTFTDYYINWVRQAPGQGLEWMGRIYPGNGNTYHNEKFKGRVTITADESTSTAYMELSSLRSEDTAVYYCAREGLYYDYDRYFDYWGQGTLVTVSS

SEQ ID NO: 1959 - protein, 122 aa, synthetic construct
QVQLVQSGAEVKKPGSSVKVSCKASGYTFTDYYINWVRQAPGQGLEWIGRIYPGNGNTYHNEKFKGRVTITADESTSTAYMELSSLRSEDTAVYYCAREGLYYDYDRYFDYWGQGTLVTVSS

SEQ ID NO: 1960 - protein, 122 aa, synthetic construct
QVQLVQSGAEVKKPGSSVKVSCKASGYTFTDYYINWVRQAPGQGLEWIGRIYPGNGNTYHNEKFKGRVTITADKSTSTAYMELSSLRSEDTAVYYCAREGLYYDYDRYFDYWGQGTLVTVSS

SEQ ID NO: 1961 - protein, 122 aa, synthetic construct
QVQLVQSGAEVKKPGSSVKVSCKASGYTFTDYYINWVRQAPGQGLEWIGRIYPGNGNTYHNEKFKGRATLTADKSTSTAYMELSSLRSEDTAVYYCAREGLYYDYDRYFDYWGQGTLVTVSS

SEQ ID NO: 1962 - protein, 122 aa, synthetic construct
QVQLVQSGAEVKKPGSSVKVSCKASGYTFTDYYINWVRQAPGQGLEWMGRIYPGNGNTYHNEKFKGRATLTADESTSTAYMELSSLRSEDTAVYYCAREGLYYDYDRYFDYWGQGTLVTVSS

SEQ ID NO: 1963 - protein, 122 aa, synthetic construct
QVQLVQSGAEVVKPGSSVKVSCKASGYTFTDYYINWVKQAPGQGLEWIGRIYPGNGNTYHNEKFKGRATLTADESTSTAYMELSSLRSEDTAVYYCAREGLYYDYDRYFDYWGQGTLVTVSS

SEQ ID NO: 1964 - protein, 122 aa, synthetic construct
QVQLVQSGAEVKKPGSSVKVSCKASGYTFTDYYINWVRQAPGQGLEWIARIYPGNGNTYHNEKFKGRATLTADKSTSTAYMELSSLRSEDTAVYYCAREGLYYDYDRYFDYWGQGTLVTVSS

SEQ ID NO: 1965 - protein, 122 aa, synthetic construct
QVQLVQSGAEVKKPGASVKVSCKASGYTFTDYYINWVRQAPGQRLEWIARIYPGNGNTYHNEKFKGRATLTRDKSASTAYMELSSLRSEDTAVYYCAREGLYYDYDRYFDYWGQGTLVTVSS

SEQ ID NO: 1966 - protein, 111 aa, synthetic construct; identical to SEQ ID NO: 8

SEQ ID NO: 1967 - protein, 111 aa, synthetic construct
DIVMTQSPDSLAVSLGERATINCRASESVDTYGNSFMHWYQQKPGQPPKLLIYLASNLESGVPDRFSGSGSGTDFTLTISSLQAEDVAVYYCHQNNEDPPAFGGGTKVEIK

SEQ ID NO: 1968 - protein, 111 aa, synthetic construct
DIVLTQSPDSLAVSLGERATINCRASESVDTYGNSFMHWYQQKPGQPPKLLIYLASNLESGVPDRFSGSGSGTDFTLTISSVQAEDVAVYYCHQNNEDPPAFGGGTKVEIK

SEQ ID NO: 1969 - protein, 111 aa, synthetic construct
DIVLTQSPDSLAVSLGERATINCRASESVDTYGNSFMHWYQQKPGQPPKLLIYLASNLESGVPDRFSGSGSRTDFTLTISSLQAEDVAVYYCHQNNEDPPAFGGGTKVEIK

SEQ ID NO: 1970 - protein, 111 aa, synthetic construct
DIQMTQSPSSLSASVGDRVTITCRASESVDTYGNSFMHWYQQKPGKAPKLLIYLASNLESGVPSRFSGSGSGTDFTLTISSLQPEDFATYYCHQNNEDPPAFGGGTKVEIK

SEQ ID NO: 1971 - protein, 111 aa, synthetic construct
DIQLTQSPSSLSASVGDRVTITCRASESVDTYGNSFMHWYQQKPGKAPKLLIYLASNLESGVPSRFSGSGSGTDFTLTISSVQPEDFATYYCHQNNEDPPAFGGGTKVEIK

SEQ ID NO: 1972 - protein, 111 aa, synthetic construct
EIVLTQSPATLSLSPGERATLSCRASESVDTYGNSFMHWYQQKPGQAPRLLIYLASNLESGIPARFSGSGSGTDFTLTISSLEPEDFAVYYCHQNNEDPPAFGGGTKVEIK

SEQ ID NO: 1973 - protein, 111 aa, synthetic construct
EIVLTQSPATLSVSPGERATLSCRASESVDTYGNSFMHWYQQKPGQAPRLLIYLASNLESGVPARFSGSGSGTDFTLTISSVEPEDFAVYYCHQNNEDPPAFGGGTKVEIK

SEQ ID NO: 1974 - protein, 111 aa, synthetic construct
DVVMTQSPLSLPVTLGQPASISCRASESVDTYGNSFMHWFQQRPGQSPRRLIYLASNLESGVPDRFSGSGSGTDFTLKISRVEAEDVGVYYCHQNNEDPPAFGGGTKVEIK

SEQ ID NO: 1975 - protein, 111 aa, synthetic construct
DVVLTQSPLSLPVTLGQPASISCRASESVDTYGNSFMHWFQQRPGQSPRLLIYLASNLESGVPDRFSGSGSGTDFTLKISRVEAEDVGVYYCHQNNEDPPAFGGGTKVEIK

SEQ ID NO: 1976 - protein, 111 aa, synthetic construct
DIVLTQSPLSLPVTLGQPASISCRASESVDTYGNSFMHWYQQRPGQSPRLLIYLASNLESGVPDRFSGSGSRTDFTLKISRVEAEDVGVYYCHQNNEDPPAFGGGTKVEIK

SEQ ID NO: 1977 - protein, 118 aa, synthetic construct; identical to SEQ ID NO: 3

SEQ ID NO: 1978 - protein, 118 aa, synthetic construct
QVQLVQSGAEVKKPGSSVKVSCKASGYTFTDYGVIWVRQAPGQGLEWMGWINTYTGEPTYADDLKGRVTITADESTSTAYMELSSLRSEDTAVYYCARRYGPEDIDYWGQGTTVTVSS

SEQ ID NO: 1979 - protein, 118 aa, synthetic construct
QIQLVQSGAEVKKPGSSVKVSCKASGYTFTDYGVIWVRQAPGQGLEWMGWINTYTGEPTYADDLKGRFTITLDESTSTAYMELSSLRSEDTAVYYCARRYGPEDIDYWGQGTTVTVSS

SEQ ID NO: 1980 - protein, 118 aa, synthetic construct
QIQLVQSGAEVKKPGSSVKVSCKASGYTFTDYGVIWVRQAPGQGLKWMGWINTYTGEPTYADDLKGRFTFTLDESTSTAYMELSSLRSEDTAVYYCARRYGPEDIDYWGQGTTVTVSS

SEQ ID NO: 1981 - protein, 118 aa, synthetic construct
QIQLVQSGAEVKKPGSSVKVSCKASGYTFTDYGVIWVRQAPGKALKWMGWINTYTGEPTYADDLKGRVTFTADESTSTAYMELSNLRSEDTAVYFCARRYGPEDIDYWGQGTTVTVSS

SEQ ID NO: 1982 - protein, 118 aa, synthetic construct
QVQLVQSGAEVKKPGSSVKVSCKASGYTFTDYGVIWVRQAPGQGLEWMGWINTYTGEPTYADDLKGRVTFTADESTSTAYMELSSLRSEDTAVYYCARRYGPEDIDYWGQGTTVTVSS

SEQ ID NO: 1983 - protein, 118 aa, synthetic construct
QVQLVQSGAEVKKPGSSVKVSCKASGYTFTDYGVIWVRQAPGQGLKWMGWINTYTGEPTYADDLKGRVTITADESTSTAYMELSSLRSEDTAVYYCARRYGPEDIDYWGQGTTVTVSS

SEQ ID NO: 1984 - protein, 118 aa, synthetic construct
QVQLVQSGAEVKKPGSSVKVSCKASGYTFTDYGVIWVRQAPGQGLKWMGWINTYTGEPTYADDLKGRVTFTADESTSTAYMELSSLRSEDTAVYYCARRYGPEDIDYWGQGTTVTVSS

SEQ ID NO: 1985 - protein, 118 aa, synthetic construct
QIQLVQSGAEVKKPGSSVKVSCKASGYTFTDYGVIWVRQAPGQGLKWMGWINTYTGEPTYADDLKGRVTFTADESTSTAYMELSSLRSEDTAVYYCARRYGPEDIDYWGQGTTVTVSS

SEQ ID NO: 1986 - protein, 118 aa, synthetic construct
QVQLVQSGAEVKKPGATVKISCKVSGYTFTDYGVIWMQQAPGKGLEWIGWINTYTGEPTYADDLKGRVTLTADTSTDTAYMELSSLRSEDTAVYFCARRYGPEDIDYWGQGTMVTVSS

SEQ ID NO: 1987 - protein, 118 aa, synthetic construct
QIQLVQSGAEVKKPGATVKISCKVSGYTFTDYGVIWMQQAPGKGLEWIGWINTYTGEPTYADDLKGRFTFTADTSTDTAYMELSSLRSEDTAVYFCARRYGPEDIDYWGQGTMVTVSS

SEQ ID NO: 1988 - protein, 118 aa, synthetic construct
QIQLVQSGAEVKKPGATVKISCKVSGYTFTDYGVIWMQQAPGKGLEWIGWINTYTGEPTYADDLKGRFTFTADTSTDTAYLELSSLRSEDTAVYFCARRYGPEDIDYWGQGTMVTVSS

SEQ ID NO: 1989 - protein, 111 aa, synthetic construct; identical to SEQ ID NO: 4

SEQ ID NO: 1990 - protein, 111 aa, synthetic construct
DVVMTQSPLSLPVTLGQPASISCRASESVDNYGISFMSWFQQRPGQSPRRLIYAASHQGSGVPDRFSGSGSGTDFTLKISRVEAEDVGVYYCLQSKEVPWTFGGGTKVEIK

SEQ ID NO: 1991 - protein, 111 aa, synthetic construct
DIVLTQSPLSLPVTLGQPASISCRASESVDNYGISFMSWFQQRPGQSPRLLIYAASHQGSGVPDRFSGSGSGTDFTLKISRVEAEDVGVYYCLQSKEVPWTFGGGTKVEIK

SEQ ID NO: 1992 - protein, 111 aa, synthetic construct
DIVLTQSPLSLPVTLGQPASISCRASESVDNYGISFMSWFQQRPGQSPRLLIYAASHQGSGVPDRFSGSGSGTDFTLKISRVEAEDVGVYFCLQSKEVPWTFGGGTKVEIK

SEQ ID NO: 1993 - protein, 111 aa, synthetic construct
DIVMTQSPLSLPVTLGQPASISCRASESVDNYGISFMSWFQQRPGQSPRLLIYAASHQGSGVPDRFSGSGSGTDFTLKISRVEAEDVGVYYCLQSKEVPWTFGGGTKVEIK

SEQ ID NO: 1994 - protein, 111 aa, synthetic construct
DVVLTQSPLSLPVTLGQPASISCRASESVDNYGISFMSWFQQRPGQSPRLLIYAASHQGSGVPDRFSGSGSGTDFTLKISRVEAEDVGVYYCLQSKEVPWTFGGGTKVEIK

SEQ ID NO: 1995 - protein, 111 aa, synthetic construct
DVVLTQSPLSLPVTLGQPASISCRASESVDNYGISFMSWFQQRPGQSPRRLIYAASHQGSGVPDRFSGSGSGTDFTLKISRVEAEDVGVYYCLQSKEVPWTFGGGTKVEIK

SEQ ID NO: 1996 - protein, 111 aa, synthetic construct
DVVMTQSPLSLPVTLGQPASISCRASESVDNYGISFMSWFQQRPGQSPRLLIYAASHQGSGVPDRFSGSGSGTDFTLKISRVEAEDVGVYYCLQSKEVPWTFGGGTKVEIK

SEQ ID NO: 1997 - protein, 111 aa, synthetic construct
DVVLTQSPLSLPVTLGQPASISCRASESVDNYGISFMSWFQQRPGQSPRLLIYAASHQGSGVPDRFSGSGSGTDFTLKISRVEAEDVGVYFCLQSKEVPWTFGGGTKVEIK

SEQ ID NO: 1998 - protein, 111 aa, synthetic construct
DVVMTQSPLSLPVTLGQPASISCRASESVDNYGISFMSWFQQRPGQSPRLLIYAASHQGSGVPDRFSGSGSGTDFTLKISRVEAEDVGVYFCLQSKEVPWTFGGGTKVEIK

SEQ ID NO: 1999 - protein, 111 aa, synthetic construct
DIELTQSPASLAVSLGQRATISCRASESVDNYGISFMSWYQQKPGQPPKLLIYAASHQGSGIPVRFSGSGSGTDFTLNIHPVEEEDAATYYCLQSKEVPWTFGSGTKLEIK

SEQ ID NO: 2000 - protein, 111 aa, synthetic construct
DIVLTQSPASLAVSLGQRATISCRASESVDNYGISFMSWYQQKPGQPPKLLIYAASHQGSGIPVRFSGSGSGTDFTLNIHPVEEEDAATYYCLQSKEVPWTFGSGTKLEIK

SEQ ID NO: 2001-2008 - skipped (8 entries)

SEQ ID NO: 2009 - protein, 470 aa, synthetic construct
MDMRVPAQLLGLLLLWLRGARCQVQLVQSGAEVKKPGSSVKVSCKASGYTFTDYGVIWVRQAPGQGLEWMGWINTYTGEPTYADDLKGRVTFTADESTSTAYMELSSLRSEDTAVYYCARRYGPEDIDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2010 - protein, 448 aa, synthetic construct
QVQLVQSGAEVKKPGSSVKVSCKASGYTFTDYGVIWVRQAPGQGLEWMGWINTYTGEPTYADDLKGRVTFTADESTSTAYMELSSLRSEDTAVYYCARRYGPEDIDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2011 - protein, 240 aa, synthetic construct
MDMRVPAQLLGLLLLWLRGARCDVVLTQSPLSLPVTLGQPASISCRASESVDNYGISFMSWFQQRPGQSPRLLIYAASHQGSGVPDRFSGSGSGTDFTLKISRVEAEDVGVYFCLQSKEVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2012 - protein, 218 aa, synthetic construct
DVVLTQSPLSLPVTLGQPASISCRASESVDNYGISFMSWFQQRPGQSPRLLIYAASHQGSGVPDRFSGSGSGTDFTLKISRVEAEDVGVYFCLQSKEVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC